(19) United States
(12) Patent Application Publication
(10) Pub. No.: US 2024/0209343 A1
(43) Pub. Date: Jun. 27, 2024

(54) ENGINEERED RNA LIGASE VARIANTS

(71) Applicant: Codexis, Inc., Redwood City, CA (US)

(72) Inventors: Rachelle Copeland, Redwood City, CA (US); David Elgart, San Mateo, CA (US); Jason Fell, Martinez, CA (US); Jovana Nazor, Milpitas, CA (US); Leland Ken Wong, San Bruno, CA (US); Zhe Rui, Newark, CA (US)

(21) Appl. No.: 18/395,294

(22) Filed: Dec. 22, 2023

Related U.S. Application Data
(60) Provisional application No. 63/476,912, filed on Dec. 22, 2022.

(51) Int. Cl. C12N 9/00; C12Q 1/6813

(52) U.S. Cl. CPC C12N 9/93; C12Q 1/6813; C12Y 605/01003

(57) ABSTRACT

The present disclosure relates to engineered RNA ligase polypeptides and compositions thereof, as well as polynucleotides encoding the engineered RNA ligase polypeptides. The present disclosure also provides methods of using the engineered RNA ligase polypeptides or compositions thereof for molecular biological, diagnostic, and other purposes.

### CROSS-REFERENCE TO RELATED APPLICATIONS

This application claims the benefit of U.S. Provisional Application No. 63/476,912, filed Dec. 22, 2022, which is incorporated by reference herein.

### REFERENCE TO SEQUENCE LISTING, TABLE OR COMPUTER PROGRAM

The Sequence Listing concurrently submitted herewith as file name CX9-231US1_ST26.xml, created on Dec. 22, 2023, with a file size of 1,145,043 bytes, is part of the specification and is incorporated by reference herein.

### TECHNICAL FIELD

The present disclosure provides engineered RNA ligase polypeptides and compositions thereof, as well as polynucleotides encoding the engineered RNA ligase polypeptides. The disclosure also provides methods of using the recombinant RNA ligase or compositions thereof, including for the synthesis of polynucleotides, as molecular biological tools, and other purposes.

### BACKGROUND

RNA ligases are a family of enzymes that catalyze the joining of pieces of RNA or DNA that are adjacent to each other. They are involved in the editing and repair of RNA, and therefore are essential proteins for biological processes. RNA ligases find uses in the profiling of target sequences, the quantification of different RNA species, the detection of particular RNA mutations, and the synthesis of polynucleotides. Based on their substrate preference, the known RNA ligases are categorized into two groups. Single stranded RNA ligases, also referred to as RNA ligase 1 or RNA ligase I, are capable of joining two RNA or DNA without a hybridizing template. Double stranded RNA ligases, also referred to as RNA ligase 2 or RNA ligase II, preferentially join a nick in an RNA duplex. RNA ligase 1 type proteins have been identified in fungi, baculoviruses, archea, and archeal viruses (thermostable properties). RNA ligase 2 type enzymes have been identified in Vibrio phage KVP40, baculoviruses and entomopoxviruses, some parasites, and archaea species.

Prototypical RNA ligases are those from bacteriophage T4. The T4 RNA Ligase 1 ligates a 5′-phosphoryl nucleic acid donor to a 3′-hydroxyl nucleic acid acceptor by connecting them with a phosphodiester bond in an ATP dependent reaction. Substrates for T4 RNA Ligase 1 include ssRNA, ssDNA, and dinucleoside pyrophosphates. T4 RNA ligase 1 is used for ligation of ssRNA and DNA, RNA ligase-mediated rapid amplification of cDNA ends (RLM-RACE), ligation of oligonucleotide adapters to cDNA or single-stranded primer extension products for PCR, oligonucleotide synthesis, and various 5′ nucleotide modifications of nucleic acids. T4 RNA ligase 2 ligates adjacent 5′ phosphate at the end of RNA and DNA strands to 3′ OH of RNA strands in the context of nicked dsRNA or RNA/DNA hybrids. T4 RNA ligase 2 exhibits significant homology to DNA ligases and mRNA capping enzymes, and is used to seal nicks in dsRNA and dsRNA/DNA hybrids. While T4 RNA ligases can act on RNA and DNA, RNA is the preferred substrate, with DNA being a less effective acceptor in the ligation reaction.

While T4 RNA ligases have become useful tools for labeling, circularizing, and ligating RNA and RNA/DNA, desirable are ligases that provide more facile and efficient ligation of polynucleotide substrates, including improved ligation of polynucleotides that contain nucleotide analogs, such as modified sugar residues and non-standard internucleotide linkages.

### SUMMARY

The present disclosure provides engineered RNA ligase polypeptides and compositions thereof, as well as polynucleotides encoding the engineered RNA ligase polypeptides. The present disclosure also provides methods of using the engineered RNA ligase polypeptides and compositions thereof for nucleic acid synthesis, diagnostic assays, and other purposes.

In one aspect, the present disclosure provides an engineered RNA ligase, or a functional fragment thereof, comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 346 of an even-numbered SEQ ID NO. of SEQ ID NOs: 14 and 24-582, or to a reference sequence corresponding to an even-numbered SEQ ID NO. of SEQ ID NOs: 14 and 24-582, wherein the amino acid sequence comprises one or more substitutions relative to the reference sequence corresponding to residues 12 to 346 of SEQ ID NO: 14, 42, or 204, or relative to the reference sequence corresponding to SEQ ID NO: 14, 42, or 204.

In some embodiments, the engineered RNA ligase comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 14, 42, or 204, or to the reference sequence corresponding to SEQ ID NO: 14, 42, or 204, wherein the amino acid sequence comprises one or more substitutions relative to the reference sequence corresponding to residues 12 to 346 of SEQ ID NO: 14, 42, or 204, or relative to the reference sequence corresponding to SEQ ID NO: 14, 42, or 204.

In some embodiments, the engineered RNA ligase comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 14, or to the reference sequence corresponding to SEQ ID NO: 14, wherein the amino acid sequence comprises one or more substitutions relative to the reference sequence corresponding to residues 12 to 346 of SEQ ID NO: 14, or relative to the reference sequence corresponding to SEQ ID NO: 14.

In some embodiments, the engineered RNA ligase comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 42 or 204, or to the reference sequence corresponding to SEQ ID NO: 42 or 204, wherein the amino acid sequence comprises one or more substitutions relative to the reference sequence corresponding to residues 12 to 346 of SEQ ID NO: 14, or relative to the reference sequence corresponding to SEQ ID NO: 14.

In some embodiments, the engineered RNA ligase comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 24-582, or to the reference sequence corresponding to an even-numbered SEQ ID NO. of SEQ ID NOs: 24-582, wherein the amino acid sequence comprises one or more substitutions relative to the reference sequence corresponding to residues 12 to 346 of SEQ ID NO: 14, or relative to the reference sequence corresponding to SEQ ID NO: 14.

In some embodiments, the engineered RNA ligase comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 24-128, or to the reference sequence corresponding to an even-numbered SEQ ID NO. of SEQ ID NOs: 24-128, wherein the amino acid sequence comprises one or more substitutions relative to the reference sequence corresponding to residues 12 to 346 of SEQ ID NO: 14, or relative to the reference sequence corresponding to SEQ ID NO: 14.

In some embodiments, the amino acid sequence of the engineered RNA ligase comprises at least a substitution at amino acid position 2, 14, 15, 18, 33, 39, 63, 69, 81, 83, 85, 86, 89, 95, 116, 117, 119, 138, 142, 144, 149, 154, 156, 159, 171, 175, 177, 181, 185, 186, 195, 202, 212, 214, 224, 226, 230, 247, 256, 257, 260, 266, 275, 280, 283, 285, 288, 291, 296, 303, 306, 307, 310, 314, 315, 316, 317, 326, 330, 331, 333, 334, 335, 337, 339, 342, or 345, or combinations thereof, wherein the amino acid positions are relative to the reference sequence corresponding to residues 12 to 346 of SEQ ID NO: 14, or relative to the reference sequence corresponding to SEQ ID NO: 14.

In some embodiments, the amino acid sequence of the engineered RNA ligase comprises at least a substitution at amino acid position 95 or 177, or combination thereof, wherein the amino acid positions are relative to the reference sequence corresponding to residues 12 to 346 of SEQ ID NO: 14, or relative to the reference sequence corresponding to SEQ ID NO: 14.

In some embodiments, the amino acid sequence of the engineered RNA ligase comprises at least a substitution at amino acid position 15, 95, 149, 154, 156, 177, 186, 195, 224, or 226, or combinations thereof, wherein the amino acid positions are relative to the reference sequence corresponding to residues 12 to 346 of SEQ ID NO: 14, or relative to the reference sequence corresponding to SEQ ID NO: 14.

In some embodiments, the amino acid sequence of the engineered RNA ligase comprises at least a substitution or substitution set at amino acid position(s) 177/260/307/345, 171/175/183/280/283/303, 95/159/177/260/288, 175/183/247/280/283/296, 177/307, 81/171/183/335, 175/183, 81/175/183/280/296/335/339, 81/183/247/266, 95/177, 171/280/283/296/303, 85/177/260/288/333/345, 177/291/307/333, 81/171/175/183/316/337, 81/171/266/280/283/335/339, 177, 95/159/177/260/280/288/306/345, 171/175/335, 177/260/30/333, 14/95/177/288/307, 95/177/345, 95/159/177/260/285/288, 266/296, 81/283/335/337, 95/159/177/260/342/345, 63/171/175/183/266/280/296/316, 280/335/337, 81/171/280, 81/171/175/296/316, 171/175/316/335, 171/280/283/296/316, 171/283/335, 63/171/175/183/266/283/303/316, 171/247/266/283/296/303/316/335, 63/81/171/175/183/266/280/283/337, 81/171/303/316/335, 288/307, 14/95/159/177/345, 81/171/175/183/247/266/280/283, 81/247/266, 280, 85/177/260, 63/183, 175/183/247/280/283/316, 291, 247, 171/280/283, 81/171/175/280/316, 333, 171/296/316, 81/171, or 280/316/335, wherein the amino acid positions are relative to the reference sequence corresponding to residues 12 to 346 of SEQ ID NO: 14, or relative to the reference sequence corresponding to SEQ ID NO: 14.

In some embodiments, the amino acid sequence of the engineered RNA ligase comprises at least one substitution provided in Tables 5.1, 6.1, 7.1, 8.1, 9.1, 10.1, 11.1, and 12.1, wherein the amino acid positions are relative to the reference sequence corresponding to residues 12 to 346 of SEQ ID NO: 14, or relative to the reference sequence corresponding to SEQ ID NO: 14.

In some embodiments, the amino acid sequence of the engineered RNA ligase comprises at least a substitution or substitution set of an RNA ligase variant provided in Tables 5.1, 6.1, 7.1, 8.1, 9.1, 10.1, 11.1, and 12.1, wherein the amino acid positions are relative to the reference sequence corresponding to residues 12 to 346 of SEQ ID NO: 14, or relative to the reference sequence corresponding to SEQ ID NO: 14.

In some embodiments, the engineered RNA ligase comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 42 or 204, or to the reference sequence corresponding to SEQ ID NO: 42 or 204.

In some embodiments, the engineered RNA ligase comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 24-582, or to the reference sequence corresponding to an even-numbered SEQ ID NO. of SEQ ID NOs: 24-582.

In some embodiments, the engineered RNA ligase comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 42 or 204, or to the reference sequence corresponding to SEQ ID NO: 42 or 204, wherein the amino acid sequence comprises one or more substitutions relative to the reference sequence corresponding to residues 12 to 346 of SEQ ID NO: 42 or 204, or relative to the reference sequence corresponding to SEQ ID NO: 42 or 204.

In some embodiments, the engineered RNA ligase comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 24-582, or to the reference sequence corresponding to an even-numbered SEQ ID NO. of SEQ ID NOs: 24-582, wherein the amino acid sequence comprises one or more substitutions relative to the reference sequence corresponding to residues 12 to 346 of SEQ ID NO: 42 or 204, or relative to the reference sequence corresponding to SEQ ID NO: 42 or 204.

In some embodiments, the amino acid sequence of the engineered RNA ligase comprises at least a substitution at amino acid position 2, 14, 15, 18, 33, 39, 63, 69, 81, 83, 85, 86, 89, 95, 116, 117, 119, 138, 142, 144, 149, 154, 156, 159, 171, 175, 177, 181, 185, 186, 195, 202, 212, 214, 224, 226, 230, 247, 256, 257, 260, 266, 275, 280, 283, 285, 288, 291, 296, 303, 306, 307, 310, 314, 315, 316, 317, 326, 330, 331, 333, 334, 335, 337, 339, 342, or 345, or combinations thereof, wherein the amino acid positions are relative to the reference sequence corresponding to residues 12 to 346 of SEQ ID NO: 42 or 204, or relative to the reference sequence corresponding to SEQ ID NO: 42 or 204.

In some embodiments, the amino acid sequence of the engineered RNA ligase comprises at least a substitution at amino acid position 95 or 177, or combination thereof, wherein the amino acid positions are relative to the reference sequence corresponding to residues 12 to 346 of SEQ ID NO: 42 or 204, or relative to the reference sequence corresponding to SEQ ID NO: 42 or 204.

In some embodiments, the amino acid sequence of the engineered RNA ligase comprises at least a substitution at amino acid position 15, 95, 149, 154, 156, 177, 186, 195, 224, or 226, or combinations thereof, wherein the amino acid positions are relative to the reference sequence corresponding to residues 12 to 346 of SEQ ID NO: 42 or 204, or relative to the reference sequence corresponding to SEQ ID NO: 42 or 204.

In some embodiments, the engineered RNA ligase comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 42, or to the reference sequence corresponding to SEQ ID NO: 42, wherein the amino acid sequence comprises one or more substitutions relative to the reference sequence corresponding to residues 12 to 346 of SEQ ID NO: 42, or relative to the reference sequence corresponding to SEQ ID NO: 42.

In some embodiments, the engineered RNA ligase comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 130-478, or to the reference sequence corresponding to an even-numbered SEQ ID NO. of SEQ ID NOs: 130-478, wherein the amino acid sequence comprises one or more substitutions relative to the reference sequence corresponding to residues 12 to 346 of SEQ ID NO: 42, or relative to the reference sequence corresponding to SEQ ID NO: 42.

In some embodiments, the amino acid sequence of the engineered RNA ligase comprises at least a substitution or substitution set at amino acid position(s) 149/156/195, 15/149/186/224/317, 15/156/195/224/226/317/331, 15/195/317/331, 149/224/331, 15/195/224/226/317/331, 15/33/86/149/154/195/317, 15/86/154/156/186/195/224/226/331, 15/149/224/317/331, 15/154/156/186/317/331, 15/33/149/224/226/331, 15/186/224/226/317/331, 15/33/149/186/224/331, 195/224/331, 15/86/156/186/195/226/317/331, 15/195/22/331, 156/186/224/226/331, 15/149/154/156/224/226, 33/156/186/195/331, 15/86/149/154/156/317/331, 15/186/317/331, 15/86/149/154/195/331, 15/33/149/154/156/226, 15/149/154/186/317/331, 195/331, 15/86/156/186/195/331, 149/156/224/226/331, 15/149/154/156/186/317/331, 15/154/186/195, 15/86/195/224/226, 230, 15/33/156/331, 15/33/156/195/224/226, 33/149/156/317, 15/33/186/195, 334, 15/154/156/224/317, 15/149/186/331, 15/86/149/186/195/317, 15/33/154/195, 15/156/186/331, 15/149/186/317, 15/33/156/186/214/224/331, 15/86/186/224/226/317, 15/154/331, 15/149/154/226, 15/33/195/226, 15/224/317/331, 15/86/186/195/317, 226/317/331, 330, 89, 15/149/186, 156/195/331, 156, 15/86/156/186/331, 275, 15/33/186/224, 15/186/317, 15/33/186/226/317, 15/149/154/156/331, 15/154/226/317, 15/86/156/186/195/317, 257, 15/154/156/186, 33/186/224, 335, 15/86/156/186/195/224/331, 154/156/317, 39, 2, 15/224/226/331, 15/33/149/224, 89/307, 15/154/156/317, 15/186/331, 15/33/154/317, 15/33/195/224/317, 15/156/224/226, 154/186/331, 185, 15/18/149/186/195, 86/149/154/156, 15/33/149/331, 326, 202, 119, 212, 15/33/156/317, 280, 142, 15/149, 15/33/86/156/195, 315, 256, 15/33/156/186, 138, 117, 116, 316, 69, 310, 317, 15/195/331, 15/33/331, 314, or 144, wherein the amino acid positions are relative to the reference sequence corresponding to residues 12 to 346 of SEQ ID NO: 42, or relative to the reference sequence corresponding to SEQ ID NO: 42.

In some embodiments, the amino acid sequence of the engineered RNA ligase comprises at least a substitution or substitution set at amino acid position(s) 15/149/154/156/186/195/331, 149/154/186/195/224/317/331, 15/33/149/154/186/195, 149/156/186/195/224/226/317, 15/149/156/195/224/226, 15/33/86/149/154/156/186/195/226/331, 15/149/156/186/195/224/317/331, 15/33/149/154/195/317, 15/149/154/195/224/317, 15/149/156/186/224/226/317/331, 15/33/149/154/156/186/195/224/226/317/331, 149/195/224, 86/149/154/156/186/195/331, 15/33/149/156/195/224/317/331, 15/149/154/156/186/195/317/331, 15/33/149/154/156/186/224/331, 15/149/154/156/195/224, 15/149/154/156/195/224/226, 15/149/154/156/186/195/224/317, 33/149/156/186/224/331, 15/33/86/149/195/224/331, 15/86/149/154/156/186/195/331, 149/186/195/331, 15/154/156/186/226/331, 15/149/154/186/195/224/226, 15/149/154/156/195/224/331, 15/33/149/154/156/195/317/331, 15/33/154/156/186/195/224/317/331, 15/149/186/195/224/331, 15/33/149/154/156/195/224/331, 15/149/154/156/224/226/317/331, 15/33/149/156/195/331, 15/149/154/156/186/224/226/331, 154/156/195/224/226, 15/33/86/149/154/156/186/195/224/317/331, 15/149/154/156/186/195/224/226, 15/33/149/195/317/331, 15/149/154/224/331, 149/154/156/195/224/226/331, 15/149/154/195/331, 15/149/154/156/186/195, 15/33/149/154/156/186/195, 15/33/86/149/154/195/224/317/331, or 149/186/195/224/226/331, wherein the amino acid positions are relative to the reference sequence corresponding to residues 12 to 346 of SEQ ID NO: 42, or relative to the reference sequence corresponding to SEQ ID NO: 42.

In some embodiments, the engineered RNA ligase comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 204, or to the reference sequence corresponding to SEQ ID NO: 204, wherein the amino acid sequence comprises one or more substitutions relative to the reference sequence corresponding to residues 12 to 346 of SEQ ID NO: 204, or relative to the reference sequence corresponding to SEQ ID NO: 204.

In some embodiments, the engineered RNA ligase comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 480-582, to the reference sequence corresponding to an even-numbered SEQ ID NO. of SEQ ID NOs: 480-582, wherein the amino acid sequence comprises one or more substitutions relative to the reference sequence corresponding to residues 12 to 346 of SEQ ID NO: 204, or relative to the reference sequence corresponding to SEQ ID NO: 204.

In some embodiments, the amino acid sequence of the engineered RNA ligase comprises at least a substitution or substitution set at amino acid position(s) 2/39/69/144/156/316/330, 2/39/69/119/185/316, 69/212/256/330, 39/119/256/316/326/330, 69/116/119/185/330, 2/39/119/144/156/159/202/256/316/326/330, 81/183/185/230, 2/39/69/116/144/156/185, 2/14/39/69/116/119/159/185/256/326, 2/116/119/316, 116/119/144/185/314/316, 39/69/116/144/181/185/256/330, 2/39/69/119/144/156/185/314, 14/39/69/144/156/212/256/314/316/326/330, 39/69/144/156/185/316/330, 156/185/334/335, 39/63/156/230/257/275/330/335, 156/266/316/330/334/335, 39/63/156/183/185/230/330/334, 69/144/316, 116/119/156/202/212/326/330, 39/69/119/138/181/185/316/326, 39/81/156/230, 2/14/69/116/119/144/159/326/330, 39/156/334/345, 230/266/334, 2/116/144/156/159, 156/183/185/230, 2/14/116/316/326/330, 39/63/156/230/316/345, 69/138/144/159/185/202/316, 39/156/257/266/316/334, 156/257/275/334, 183, 89/156/185/230/316/345, 2/14/119, 144/156/316/330, 39/81/185/316/334, 89/230/266/345, 39/156/230/275/316, 39/156/230/266/334/335, 156/185/275/316/330, 119/156/202, 230/257/275/316/330/345, 230, 81/156/330/335/345, 156/183/230/266, 39/185, 39/81/183/275/316/334/345, 156/230/257/316, 39/116/156, or 183/185/257/275/316/330/334, wherein the amino acid positions are relative to the reference sequence corresponding to residues 12 to 346 of SEQ ID NO: 204, or relative to the reference sequence corresponding to SEQ ID NO: 204.

In some embodiments, the amino acid sequence of the engineered RNA ligase comprises at least one substitution provided in Tables 5.1, 6.1, 7.1, 8.1, 9.1, 10.1, 11.1, and 12.1, wherein the amino acid positions are relative to the reference sequence corresponding to residues 12 to 346 of SEQ ID NO: 14, 42, or 204, or relative to the reference sequence corresponding to SEQ ID NO: 14, 42, or 204.

In some embodiments, the amino acid sequence of the engineered RNA ligase comprises at least a substitution or substitution set of an RNA ligase variant provided in Tables 5.1, 6.1, 7.1, 8.1, 9.1, 10.1, 11.1, and 12.1, wherein the amino acid positions are relative to the reference sequence corresponding to residues 12 to 346 of SEQ ID NO: 14, 42, or 204, or relative to the reference sequence corresponding to SEQ ID NO: 14, 42, or 204.

In some embodiments, the engineered RNA ligase comprises an amino acid sequence having least 80%, 81%, 82%, 83%, 84%, 85%, 86%, 87%, 88%, 89%, 90%, 91%, 92%, 93%, 94%, 95%, 96%, 97%, 98%, 99% or more sequence identity to a reference sequence comprising a substitution or substitution set provided in Tables 5.1, 6.1, 7.1, 8.1, 9.1, 10.1, 11.1, and 12.1, wherein the amino acid positions are relative to the reference sequence corresponding to residues 12 to 346 of SEQ ID NO: 14, 42, or 204, or relative to the reference sequence corresponding to SEQ ID NO: 14, 42, or 204.

In some embodiments, the engineered RNA ligase comprises an amino acid sequence comprising residues 12 to 346 of an even numbered SEQ ID NO. of SEQ ID NOs: 14-582, or an amino acid sequence comprising an even numbered SEQ ID NO. of SEQ ID NOs: 14-582. In some embodiments, the amino acid sequence optionally has 1, 2, 3, 4, 5, 6, 7, 8, 9, or up to 10 substitutions.

In some embodiments, the engineered RNA ligase comprises an amino acid sequence comprising residues 12 to 346 of SEQ ID NO: 42 or 204, or an amino acid sequence comprising SEQ ID NO: 42 or 204. In some embodiments, the amino acid sequence optionally has 1, 2, 3, 4, 5, 6, 7, 8, 9, or up to 10 substitutions in the amino acid sequence.

In some embodiments, the engineered RNA ligase has RNA ligase 2 activity. In some embodiments, the engineered RNA ligase has RNA ligase 2 activity and at least one improved property as compared to a reference RNA ligase.

In some embodiments, the improved property of the engineered RNA ligase is selected from i) increased activity, ii) increased stability, iii) increased thermostability, iv) increased product yield, v) increased activity on polynucleotide substrates containing phosphorothioate internucleotide linkages, vi) increased activity on polynucleotide substrates containing 2′-modifications, vii) increased substrate tolerance, or any combination of i), ii), iii), iv), v), vi), and vii), compared to a reference RNA ligase. In some embodiments, the improved property of the engineered RNA ligase is in comparison to the reference RNA ligase having the sequence corresponding to residues 12 to 346 of SEQ ID NO: 14, 42, or 204, or the sequence corresponding to SEQ ID NO: 14, 42, or 204.

In some embodiments, the engineered RNA ligase is purified. In some further embodiments, the engineered RNA ligase is provided in solution, as a lyophilizate, or immobilized on a substrate, such as solid substrates, porous substrates, membranes or particles.

In another aspect, the present disclosure provides a recombinant polynucleotide comprising a polynucleotide sequence encoding the engineered RNA ligases disclosed herein.

In some embodiments, the recombinant polynucleotide comprises a polynucleotide sequence encoding an engineered RNA ligase comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 346 of an even-numbered SEQ ID NO. of SEQ ID NOs: 14 and 24-582, or to a reference sequence corresponding to an even-numbered SEQ ID NO. of SEQ ID NOs: 14 and 24-582, wherein the amino acid sequence comprises one or more substitutions relative to the reference sequence corresponding to residues 12 to 346 of SEQ ID NO: 14, 42, or 204, or relative to the reference sequence corresponding to SEQ ID NO: 14, 42, or 204.

In some embodiments, the recombinant polynucleotide comprises a polynucleotide sequence encoding an engineered RNA ligase comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 14, 42, or 204, or to the reference sequence corresponding to SEQ ID NO: 14, 42, or 204, wherein the amino acid sequence comprises one or more substitutions relative to the reference sequence corresponding to residues 12 to 346 of SEQ ID NO: 14, 42, or 204, or relative to the reference sequence corresponding to SEQ ID NO: 14, 42, or 204.

In some embodiments, the recombinant polynucleotide comprises a polynucleotide sequence encoding an engineered RNA ligase comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 14, or to the reference sequence corresponding to SEQ ID NO: 14, wherein the amino acid sequence comprises one or more substitutions relative to the reference sequence corresponding to residues 12 to 346 of SEQ ID NO: 14, or relative to the reference sequence corresponding to SEQ ID NO: 14.

In some embodiments, the recombinant polynucleotide comprises a polynucleotide sequence encoding an engineered RNA ligase comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 42, or to the reference sequence corresponding to SEQ ID NO: 42, wherein the amino acid sequence comprises one or more substitutions relative to the reference sequence corresponding to residues 12 to 346 of SEQ ID NO: 42, or relative to the reference sequence corresponding to SEQ ID NO: 42.

In some embodiments, the recombinant polynucleotide comprises a polynucleotide sequence encoding an engineered RNA ligase comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 204, or to the reference sequence corresponding to SEQ ID NO: 204, wherein the amino acid sequence comprises one or more substitutions relative to the reference sequence corresponding to residues 12 to 346 of SEQ ID NO: 204, or relative to the reference sequence corresponding to SEQ ID NO: 204.

In some embodiments, the recombinant polynucleotide comprising a polynucleotide sequence having at least 70%, 75%, 75%, 80%, 81%, 82%, 83%, 84%, 85%, 90%, 91%, 92%, 93%, 94%, 95%, 96%, 97%, 98%, 99%, or more sequence identity to a reference polynucleotide sequence corresponding to nucleotide residues 34 to 1038 of SEQ ID NO: 13, 41, or 203, or to a reference polynucleotide sequence corresponding to SEQ ID NO: 13, 41, or 203, wherein the recombinant polynucleotide encodes an RNA ligase.

In some embodiments, the recombinant polynucleotide comprises a polynucleotide sequence having at least 70%, 75%, 80%, 81%, 82%, 83%, 84%, 85%, 90%, 91%, 92%, 93%, 94%, 95%, 96%, 97%, 98%, 99%, or more sequence identity to a reference polynucleotide sequence corresponding to nucleotide residues 34 to 1038 of an odd numbered SEQ ID NO. of SEQ ID NOs: 23-581, or to a reference polynucleotide sequence corresponding an odd numbered SEQ ID NO. of SEQ ID NOs: 23-581, wherein the recombinant polynucleotide encodes an RNA ligase.

In some embodiments, the polynucleotide sequence of the recombinant polynucleotide encoding an engineered RNA ligase is codon optimized for expression in an organism or cell type thereof, for example a bacterial cell, a fungal cell, insect cell, or mammalian cell.

In some embodiments, the recombinant polynucleotide comprises a polynucleotide sequence comprising nucleotide residues 34 to 1038 of SEQ ID NO. 13, 41, or 203, or a polynucleotide sequence comprising SEQ ID NOs: 13, 41, or 203.

In some embodiments, the recombinant polynucleotide comprises a polynucleotide sequence comprising nucleotide residues 34 to 1038 of an odd numbered SEQ ID NO. of SEQ ID NOs: 23-581, or a polynucleotide sequence comprising an odd numbered SEQ ID NO. of SEQ ID NOs: 23-581.

In a further aspect, the present disclosure provides expression vectors comprising at least one recombinant polynucleotide provided herein encoding an engineered RNA ligase. In some embodiments, the recombinant polynucleotide of the expression vector is operably linked to a control sequence. In some embodiments, the control sequence comprises a promoter, particularly a heterologous promoter.

In another aspect, the present disclosure also provides a host cell comprising at least one expression vector provided herein. In some embodiments, the host cell is a prokaryotic cell or a eukaryotic cell. In some embodiments, the host cell is a bacterial cell, fungal cell, insect cell, or mammalian cell. In some preferred embodiments, the host cell is a bacterial cell, such as E. coli. or B. subtilis.

In a further aspect, the present disclosure provides a method of producing an engineered RNA ligase polypeptide, the method comprising culturing a host cell described herein under suitable culture conditions such that at least one engineered RNA ligase is produced. In some embodiments, the method further comprises recovering or isolating the engineered RNA ligase from the culture and/or host cells. In some embodiments, the method further comprises the step of purifying the engineered RNA ligase.

In another aspect, the present disclosure provides a composition comprising at least one engineered RNA ligase disclosed herein. In some embodiments, the composition comprises at least a buffer. In some embodiments, the composition further comprises a nucleotide substrate (e.g., ATP or dATP) and one or more of polynucleotide substrates, such as synthetic polynucleotides, particularly modified polynucleotides.

In a further aspect, the present disclosure provides a method of ligating at least a first polynucleotide strand and a second polynucleotide strand, comprising contacting the first polynucleotide strand and a second polynucleotide strand with an engineered RNA ligase described herein in presence of a nucleotide substrate under conditions suitable for ligation of the first polynucleotide strand to the second polynucleotide strand, wherein the first polynucleotide strand comprises a ligatable 5′-end and the second polynucleotide strand comprises a 3′-end ligatable to the 5-end of the first polynucleotide strand. In some embodiments, the first polynucleotide strand and/or second polynucleotide strand comprises RNA or a mixture of RNA and DNA.

In some embodiments, the method further comprises a third polynucleotide strand, wherein the first polynucleotide strand and second polynucleotide strand hybridize adjacent to one another on the third polynucleotide to position the 5′-end of the first polynucleotide strand adjacent to the 3′-end of the second polynucleotide strand to form a nick. In some embodiments, the third polynucleotide strand is continuous with the first polynucleotide strand or second polynucleotide strand. In some embodiments, the third polynucleotide strand is continuous with the first polynucleotide strand and second polynucleotide strand to form a single continuous polynucleotide substrate.

In some embodiments, 3′-end region of the second polynucleotide strand that hybridizes to the third polynucleotide strand is at least 4, 6, 8 or more base pairs in length. In some embodiments, the 5-end region of the first polynucleotide strand that hybridizes to the third polynucleotide strand is at least 4, 6, 8 or more base pairs in length.

In some embodiments of the method, the third polynucleotide strand comprises a splint or bridging polynucleotide, wherein the 5′-terminal sequence of the first polynucleotide strand and the 3′-terminal sequence of the second polynucleotide strand hybridize adjacent to one another on the splint or bridging polynucleotide to position the 5′-end of the first polynucleotide strand adjacent to the 3′-end of the second polynucleotide strand.

In some embodiments, the first and third polynucleotide strands hybridize to each other to form a first double stranded polynucleotide fragment, and the second polynucleotide strand hybridizes to a fourth polynucleotide strand to form a second double stranded fragment, wherein the first and second double stranded fragments have complementary ends that can base pair to form substrates for the engineered RNA ligase.

In some embodiments, the engineered RNA ligase is used in a method to synthesize RNA or DNA/RNA polynucleotides by ligation of shorter RNA or DNA/RNA oligonucleotides. In some embodiments, the engineered RNA ligase is used to ligate the 3′ OH of RNA to the 5′ phosphate of DNA or RNA. In some embodiments, the engineered RNA ligase is used to ligate the 3′ OH of RNA to the 5′ phosphate of DNA in a double-stranded-format NGS RNA library construction. In some embodiments, the engineered RNA ligase is used in a method of preparing RNA rings. In some embodiments, the engineered RNA ligase is used to repair nicks in dsRNA or dsRNA/DNA.

In a further aspect, the present disclosure also provides a kit comprising at least one engineered RNA ligase disclosed herein. In some embodiments, the kit further comprises one or more of a buffer, nucleotide substrate (e.g., ATP or dATP), and/or one or more polynucleotide substrates.

### DETAILED DESCRIPTION

The present disclosure provides engineered RNA ligase polypeptides and compositions thereof, as well as polynucleotides encoding the engineered RNA ligase polypeptides. The disclosure also provides methods of using of the engineered RNA ligase polypeptides and compositions thereof for molecular biological, diagnostic, and other purposes. In some embodiments, the engineered RNA ligase polypeptides display, among others, increased activity, increased stability, increased product yield, increased thermal stability, and/or increased activity on polynucleotides containing one or more modified nucleotides or nucleotide analogs.

### Abbreviations and Definitions

Unless defined otherwise, all technical and scientific terms used herein generally have the same meaning as commonly understood by one of ordinary skill in the art to which this invention pertains. Generally, the nomenclature used herein and the laboratory procedures of cell culture, molecular genetics, microbiology, organic chemistry, analytical chemistry and nucleic acid chemistry described below are those well-known and commonly employed in the art. Such techniques are well known and described in numerous texts and reference works well known to those of skill in the art. Standard techniques, or modifications thereof, are used for chemical syntheses and chemical analyses.

Although any suitable methods and materials similar or equivalent to those described herein find use in the practice of the present invention, exemplary methods and materials are described herein. It is to be understood that the present invention is not limited to the particular methodology, protocols, and reagents described, as these may vary, depending upon the context they are used by those of skill in the art. Accordingly, the terms defined immediately below are more fully described by reference to the application as a whole.

As used herein, the singular “a,” “an,” and “the” include the plural references, unless the context clearly indicates otherwise.

As used herein, the term “comprising” and its cognates are used in their inclusive sense (i.e., equivalent to the term “including” and its corresponding cognates).

It is also to be understood that where description of embodiments use the term “comprising” and its cognates, the embodiments can also be described using language “consisting essentially of” or “consisting of.”

Moreover, numeric ranges are inclusive of the numbers defining the range. Thus, every numerical range disclosed herein is intended to encompass every narrower numerical range that falls within such broader numerical range, as if such narrower numerical ranges were all expressly written herein. It is also intended that every maximum (or minimum) numerical limitation disclosed herein includes every lower (or higher) numerical limitation, as if such lower (or higher) numerical limitations were expressly written herein.

As used herein, the term “about” means an acceptable error for a particular value. In some instances, “about” means within 0.05%, 0.5%, 1.0%, or 2.0%, of a given value range. In some instances, “about” means within 1, 2, 3, or 4 standard deviations of a given value.

Furthermore, the headings provided herein are not limitations of the various aspects or embodiments of the invention which can be had by reference to the application as a whole. Accordingly, the terms defined immediately below are more fully defined by reference to the application as a whole.

“EC” number refers to the Enzyme Nomenclature of the Nomenclature Committee of the International Union of Biochemistry and Molecular Biology (NC-IUBMB). The IUBMB biochemical classification is a numerical classification system for enzymes based on the chemical reactions they catalyze.

“ATCC” refers to the American Type Culture Collection whose biorepository collection includes genes and strains.

“NCBI” refers to National Center for Biological Information and the sequence databases provided therein.

“Protein,” “polypeptide,” and “peptide” are used interchangeably to denote a polymer of at least two amino acids covalently linked by an amide bond, regardless of length or post-translational modification (e.g., glycosylation or phosphorylation).

“Amino acids” and “amino acid” are referred to herein by either their commonly known three-letter symbols or by the one-letter symbols recommended by IUPAC-IUB Biochemical Nomenclature Commission. The abbreviations used for the genetically encoded amino acids are conventional and are as follows: alanine (Ala or A), arginine (Arg or R), asparagine (Asn or N), aspartate (Asp or D), cysteine (Cys or C), glutamate (Glu or E), glycine (Gly or G), glutamine (Gln or Q), histidine (His or H), isoleucine (Ile or I), leucine (Leu or L), lysine (Lys or K), methionine (Met or M), phenylalanine (Phe or F), proline (Pro or P), serine (Ser or S), threonine (Thr or T), tryptophan (Trp or W), tyrosine (Tyr or Y), and valine (Val or V). When the three-letter abbreviations are used, unless specifically preceded by an “L” or a “D” or clear from the context in which the abbreviation is used, the amino acid may be in either the L- or D-configuration about α-carbon (C_{α}). For example, whereas “Ala” designates alanine without specifying the configuration about the α-carbon, “D-Ala” and “L-Ala” designate D-alanine and L-alanine, respectively. When the one-letter abbreviations are used, upper case letters designate amino acids in the L-configuration about the α-carbon and lower case letters designate amino acids in the D-configuration about the α-carbon. For example, “A” designates L-alanine and “a” designates D-alanine. When polypeptide sequences are presented as a string of one-letter or three-letter abbreviations (or mixtures thereof), the sequences are presented in the amino (N) to carboxy (C) direction in accordance with common convention.

“Fusion protein,” and “chimeric protein” and “chimera” refer to hybrid proteins created through the joining of two or more polynucleotides that originally encode separate proteins. In some embodiments, fusion proteins are created by recombinant technology (e.g., molecular biology techniques known in the art).

“RNA ligase” refers to enzymes that covalently joins the 5′-phosphoryl termini of RNA or DNA to the 3′-hydroxyl termini of RNA or DNA. Two families of RNA ligases are known to occur in nature. RNA ligase 1 catalyzes the covalent joining of single stranded 5′-phosphoryl termini of RNA or DNA to single stranded 3-hydroxyl termini of RNA or DNA. RNA ligase 2 also catalyzes the covalent joining of a 3′-hydroxyl terminus of RNA to a 5′-phosphorylated RNA or DNA but shows preference for double stranded substrates. In some embodiments, RNA ligases include those enzymes classified in EC 6.5.1.3.

Without being bound by any theory of operation, although some DNA ligases are capable of acting on either DNA or RNA as the 3′-hydroxyl strand substrate, an RNA ligase 2 acting at a duplex nick preferentially acts on 3′-hydroxyl strand of RNA but is agnostic to whether 5-phosphoryl strand is DNA or RNA. Moreover, in some embodiments, the strand with the 3′-hydroxyl may include deoxyribonucleotides if sufficient number of ribonucleotides are present at the 3′-hydroxyl terminus. For example, the RNA specificity of T4 RNA ligase 2 is affected by the 3′-hydroxyl strand, and specifically by the two terminal ribonucleotides of the 3-′hydroxyl side of the nick (see, e.g., Nandakumar et al., Mol. Cell., 2004, 16:211-221). It is to be understood that the ligation reaction is not limited to naturally occurring RNA and DNA substrates also includes polynucleotide substrates that contain modified nucleotides and/or nucleotide analogs.

“Polynucleotide,” “nucleic acid,” or “oligonucleotide” is used herein to denote a polymer comprising at least two nucleotides where the nucleotides are either deoxyribonucleotides or ribonucleotides or mixtures of deoxyribonucleotides and ribonucleotides. In some embodiments, the abbreviations used for genetically encoding nucleosides are conventional and are as follow: adenosine (A); guanosine (G); cytidine (C); thymidine (T); and uridine (U). Unless specifically delineated, the abbreviated nucleosides may be either ribonucleosides or 2′-deoxyribonucleosides. The nucleosides may be specified as being either ribonucleosides or 2′-deoxyribonucleosides on an individual basis or on an aggregate basis. When a polynucleotide, nucleic acid, or oligonucleotide sequences are presented as a string of one-letter abbreviations, the sequences are presented in the 5′ to 3′ direction in accordance with common convention, and the phosphates are not indicated. The term “DNA” refers to deoxyribonucleic acid. The term “RNA” refers to ribonucleic acid. The polynucleotide or nucleic acid may be single-stranded or double-stranded, or may include both single-stranded regions and double-stranded regions.

In some embodiments, the terms “polynucleotide,” “nucleic acid” and “oligonucleotide” encompasses polynucleotide or nucleic acid or oligonucleotide analogs, which include, among others, nucleosides linked together via other than standard phosphodiester linkages, such as non-standard linkages of phosphoramidates, phosphorothioates, amide linkages, etc.; nucleosides with modified and/or synthetic nucleobases, for example inosine, xanthine, hypoxanthine, etc.; nucleosides with modified sugar residues, such as 2′-O-alkyl, 2′-halo, 2,3-dideoxy, 2′-halo-2′deoxy, 3-D-ribo LNA, α-L-ribo-LNA (locked nucleic acids), etc.; and/or 5′-phosphate analogs, including, among others, phosphorothioate, phosphoacetate, phosphoramidate, monomethylphosphate, methylphosphonate, or phosphonocarboxylate.

“Duplex” and “ds” refer to a double-stranded nucleic acid (e.g., DNA or RNA) molecule comprised of two single-stranded polynucleotides that are complementary in their sequence (e.g., A pairs to T or U, C pairs to G), arranged in an antiparallel 5′ to 3′ orientation, and held together by hydrogen bonds between the nucleobases (e.g., adenine [A], guanine [G], cytosine [C], thymine [T], uridine [U]).

“Complementary” is used herein to describe the structural relationship between nucleotide bases that are capable of forming base pairs with one another. For example, a purine nucleotide base present on a polynucleotide that is complementary to a pyrimidine nucleotide base on a polynucleotide may base pair by forming hydrogen bonds with one another. Complementary nucleotide bases can base pair via Watson/Crick base pairing or in any other manner than forms stable duplexes or other nucleic acid structures.

“Watson/Crick Base-Pairing” refers to a pattern of specific pairs of nucleobases and analogs that bind together through sequence-specific hydrogen-bonds, e.g. A pairs with T or U, and G pairs with C.

“Annealing” or “Hybridization” refers to the base-pairing interactions of one nucleobase polymer (e.g., poly- and oligonucleotides) with another that results in the formation of a double-stranded structure, a triplex structure or a quaternary structure. Annealing or hybridization can occur via Watson-Crick base-pairing interactions, but may be mediated by other hydrogen-bonding interactions, such as Hoogsteen base pairing. In some embodiments, the nucleobase polymer that anneals or hybridizes to another is a single nucleobase polymer while in other embodiments, the nucleobase polymers are separate nucleobase polymers.

“Engineered,” “recombinant,” “non-naturally occurring,” and “variant,” when used with reference to a cell, a polynucleotide or a polypeptide refer to a material or a material corresponding to the natural or native form of the material that has been modified in a manner that would not otherwise exist in nature or is identical thereto but produced or derived from synthetic materials and/or by manipulation using recombinant techniques.

“Wild-type” and “naturally-occurring” refer to the form found in nature. For example, a wild-type polypeptide or polynucleotide sequence is a sequence present in an organism that can be isolated from a source in nature and which has not been intentionally modified by human manipulation.

“Coding sequence” and synonymously “encoding” refers to that part of a nucleic acid (e.g., a gene) that encodes an amino acid sequence of a protein.

“Percent (%) sequence identity” refers to comparisons among polynucleotides and polypeptides, and are determined by comparing two optimally aligned sequences over a comparison window, wherein the portion of the polynucleotide or polypeptide sequence in the comparison window may comprise additions or deletions (i.e., gaps) as compared to the reference sequence for optimal alignment of the two sequences. The percentage may be calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Alternatively, the percentage may be calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Those of skill in the art appreciate that there are many established algorithms available to align two sequences. Optimal alignment of sequences for comparison can be conducted, e.g., by the local homology algorithm of Smith and Waterman (Smith and Waterman, Adv. Appl. Math., 1981, 2:482), by the homology alignment algorithm of Needleman and Wunsch (Needleman and Wunsch, J. Mol. Biol., 1970, 48:443), by the search for similarity method of Pearson and Lipman (Pearson and Lipman, Proc. Natl. Acad. Sci. USA, 1988, 85:2444), by computerized implementations of these algorithms (e.g., GAP, BESTFIT, FASTA, and TFASTA in the GCG Wisconsin Software Package), or by visual inspection, as known in the art. Examples of algorithms that are suitable for determining percent sequence identity and sequence similarity include, but are not limited to the BLAST and BLAST 2.0 algorithms (see, e.g., Altschul et al., J. Mol. Biol., 1990, 215: 403-410; and Altschul et al., Nucleic Acids Res., 1977, 3389-3402). Software for performing BLAST analyses is publicly available through the National Center for Biotechnology Information website. This algorithm involves first identifying high scoring sequence pairs (HSPs) by identifying short words of length “W” in the query sequence, which either match or satisfy some positive-valued threshold score “T,” when aligned with a word of the same length in a database sequence. T is referred to as the neighborhood word score threshold (see Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters “M” (reward score for a pair of matching residues; always >0) and “N” (penalty score for mismatching residues; always <0). For amino acid sequences, a scoring matrix is used to calculate the cumulative score. Extension of the word hits in each direction are halted when: the cumulative alignment score falls off by the quantity “X” from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see, e.g., Henikoff and Henikoff, Proc. Natl. Acad. Sci. USA, 1989, 89:10915). Exemplary determination of sequence alignment and % sequence identity can employ the BESTFIT or GAP programs in the GCG Wisconsin Software package (Accelrys, Madison WI), using default parameters provided.

“Reference sequence” refers to a defined sequence used as a basis for a sequence comparison. A reference sequence may be a subset of a larger sequence, for example, a segment of a full-length gene or polypeptide sequence. Generally, a reference sequence is at least 20 nucleotide or amino acid residues in length, at least 25 residues in length, at least 50 residues in length, at least 100 residues in length or the full length of the nucleic acid or polypeptide. Since two polynucleotides or polypeptides may each (1) comprise a sequence (i.e., a portion of the complete sequence) that is similar between the two sequences, and (2) may further comprise a sequence that is divergent between the two sequences, sequence comparisons between two (or more) polynucleotides or polypeptide are typically performed by comparing sequences of the two polynucleotides or polypeptides over a “comparison window” to identify and compare local regions of sequence similarity. In some embodiments, a “reference sequence” can be based on a primary amino acid sequence, where the reference sequence is a sequence that can have one or more changes in the primary sequence. For instance, the phrase “a reference sequence corresponding to SEQ ID NO: 14, having a lysine at the residue corresponding to X14” (or “a reference sequence corresponding to SEQ ID NO: 14, having a lysine at the residue corresponding to position 14”) refers to a reference sequence in which the corresponding residue at position X14 in SEQ ID NO: 14 (e.g., an alanine), has been changed to lysine.

“Comparison window” refers to a conceptual segment of contiguous nucleotide positions or amino acids residues wherein a sequence may be compared to a reference sequence. In some embodiments, the comparison window is at least 15 to 20 contiguous nucleotides or amino acids and wherein the portion of the sequence in the comparison window may comprise additions or deletions (i.e., gaps) of 20 percent or less as compared to the reference sequence (which does not comprise additions or deletions) for optimal alignment of the two sequences. In some embodiments, the comparison window can be longer than 15-20 contiguous residues, and includes, optionally 30, 40, 50, 100, or longer windows.

“Corresponding to”, “reference to,” and “relative to” when used in the context of the numbering of a given amino acid or polynucleotide sequence refer to the numbering of the residues of a specified reference sequence when the given amino acid or polynucleotide sequence is compared to the reference sequence. In other words, the residue number or residue position of a given polymer is designated with respect to the reference sequence rather than by the actual numerical position of the residue within the given amino acid or polynucleotide sequence. For example, a given amino acid sequence, such as that of an engineered RNA ligase, can be aligned to a reference sequence by introducing gaps to optimize residue matches between the two sequences. In these cases, although the gaps are present, the numbering of the residue in the given amino acid or polynucleotide sequence is made with respect to the reference sequence to which it has been aligned. In some embodiments, the sequence is tagged (e.g., with a histidine tag).

“Mutation” refers to the alteration of a nucleic acid sequence. In some embodiments, mutations result in changes to the encoded polypeptide sequence (i.e., as compared to the original sequence without the mutation). In some embodiments, the mutation comprises a substitution, such that a different amino acid is produced. In some alternative embodiments, the mutation comprises an addition, such that an amino acid is added (e.g., insertion) to the original polypeptide sequence. In some further embodiments, the mutation comprises a deletion, such that an amino acid is deleted from the original polypeptide sequence. Any number of mutations may be present in a given sequence.

“Amino acid difference” and “residue difference” refer to a difference in the amino acid residue at a position of a polypeptide sequence relative to the amino acid residue at a corresponding position in a reference sequence. The amino acid positions of amino acid differences generally are referred to herein as “Xn,” where n refers to the corresponding position in the reference sequence upon which the residue difference is based. For example, a “residue difference at position X14 as compared to SEQ ID NO: 14” (or a “residue difference at position 14 as compared to SEQ ID NO: 14”) refers to a difference of the amino acid residue at the polypeptide position corresponding to position 14 of SEQ ID NO: 14. Thus, if the reference polypeptide of SEQ ID NO: 14 has a alanine at position 14, then a “residue difference at position X14 as compared to SEQ ID NO: 14” refers to an amino acid substitution of any residue other than alanine at the position of the polypeptide corresponding to position 14 of SEQ ID NO: 14. In some instances herein, the specific amino acid residue difference at a position is indicated as “XnY” where “Xn” specified the corresponding residue and position of the reference polypeptide (as described above), and “Y” is the single letter identifier of the amino acid found in the engineered polypeptide (i.e., the different residue than in the reference polypeptide). In some instances (e.g., in the Tables in the Examples), the present disclosure also provides specific amino acid differences denoted by the conventional notation “AnB”, where A is the single letter identifier of the residue in the reference sequence, “n” is the number of the residue position in the reference sequence, and B is the single letter identifier of the residue substitution in the sequence of the engineered polypeptide. In some embodiments, the amino acid difference, e.g., a substitution, is denoted by the abbreviation “nB,” without the identifier for the residue in the reference sequence. In some instances, an amino acid residue difference or substitution may be a deletion and may be denoted by a “−”. In some embodiments, the phrase “an amino acid residue nB” denotes the presence of the amino acid residue in the engineered polypeptide, which may or may not be a substitution in context of a reference polypeptide or amino acid sequence.

In some instances, a polypeptide of the present disclosure can include one or more amino acid residue differences relative to a reference sequence, which is indicated by a list of the specified positions where residue differences are present relative to the reference sequence. In some embodiments, where more than one amino acid can be used in a specific residue position of a polypeptide, the various amino acid residues that can be used are separated by a “/” (e.g., X39Q/X39S, X39Q/S, or 39Q/S). The present disclosure includes engineered polypeptide sequences comprising one or more amino acid differences that include either/or both conservative and non-conservative amino acid substitutions, as well as insertions and deletions of amino acids in the sequence.

“Amino acid substitution set” and “substitution set” refers to a group of amino acid substitutions within a polypeptide sequence. In some embodiments, substitution sets comprise 2, 3, 4, 5, 6, 7, 8, 9, 10, 11, 12, 13, 14, 15, or more amino acid substitutions. In some embodiments, a substitution set refers to the set of amino acid substitutions that is present in any of the variant RNA ligase polypeptides listed in any of the Tables in the Examples. In these substitution sets, the individual substitutions are separated by a semicolon (“;”; e.g., M95V;V177P) or slash (“/”; e.g., M95V/V177P or 95V/177P).

“Conservative amino acid substitution” refers to a substitution of a residue with a different residue having a similar side chain, and thus typically involves substitution of the amino acid in the polypeptide with amino acids within the same or similar defined class of amino acids. By way of example and not limitation, an amino acid with an aliphatic side chain may be substituted with another aliphatic amino acid (e.g., alanine, valine, leucine, and isoleucine); an amino acid with hydroxyl side chain is substituted with another amino acid with a hydroxyl side chain (e.g., serine and threonine); an amino acids having aromatic side chains is substituted with another amino acid having an aromatic side chain (e.g., phenylalanine, tyrosine, tryptophan, and histidine); an amino acid with a basic side chain is substituted with another amino acid with a basis side chain (e.g., lysine and arginine); an amino acid with an acidic side chain is substituted with another amino acid with an acidic side chain (e.g., aspartic acid or glutamic acid); and a hydrophobic or hydrophilic amino acid is replaced with another hydrophobic or hydrophilic amino acid, respectively.

“Non-conservative substitution” refers to substitution of an amino acid in the polypeptide with an amino acid with significantly differing side chain properties. Non-conservative substitutions may use amino acids between, rather than within, the defined groups and affect: (a) the structure of the peptide backbone in the area of the substitution (e.g., proline for glycine); (b) the charge or hydrophobicity; and/or (c) the bulk of the side chain. By way of example and not limitation, exemplary non-conservative substitutions include an acidic amino acid substituted with a basic or aliphatic amino acid; an aromatic amino acid substituted with a small amino acid; and a hydrophilic amino acid substituted with a hydrophobic amino acid.

“Deletion” refers to modification to the polypeptide by removal of one or more amino acids from the reference polypeptide. Deletions can comprise removal of 1 or more amino acids, 2 or more amino acids, 5 or more amino acids, 10 or more amino acids, 15 or more amino acids, or 20 or more amino acids, up to 10% of the total number of amino acids, or up to 20% of the total number of amino acids making up the reference polypeptide while retaining enzymatic activity and/or retaining the improved properties of an engineered RNA ligase. Deletions can be directed to the internal portions and/or terminal portions of the polypeptide. In various embodiments, the deletion can comprise a continuous segment or can be discontinuous. As noted above, deletions are indicated by “−”, and may be present in substitution sets.

“Insertion” refers to modification to the polypeptide by addition of one or more amino acids from the reference polypeptide. Insertions can be in the internal portions of the polypeptide, or to the carboxy or amino terminus. Insertions as used herein include fusion proteins as is known in the art. The insertion can be a contiguous segment of amino acids or separated by one or more of the amino acids in the naturally occurring polypeptide.

“Functional fragment” and “biologically active fragment” are used interchangeably herein, to refer to a polypeptide that has an amino-terminal and/or carboxy-terminal deletion(s) and/or internal deletions, but where the remaining amino acid sequence is identical to the corresponding positions in the sequence to which it is being compared (e.g., a full length engineered RNA ligase of the present invention) and that retains substantially all of the activity of the full-length polypeptide.

“Isolated polypeptide” refers to a polypeptide which is substantially separated from other contaminants that naturally accompany it (e.g., protein, lipids, and polynucleotides). The term embraces polypeptides which have been removed or purified from their naturally-occurring environment or expression system (e.g., host cell or in vitro synthesis). The recombinant RNA ligase polypeptides may be present within a cell, present in the cellular medium, or prepared in various forms, such as lysates or isolated preparations. As such, in some embodiments, the recombinant RNA ligase polypeptides provided herein are isolated polypeptides.

“Substantially pure polypeptide” or “purified” refers to a composition in which the polypeptide species is the predominant species present (i.e., on a molar or weight basis it is more abundant than any other individual macromolecular species in the composition), and is generally a substantially purified composition when the object species comprises at least about 50 percent of the macromolecular species present by mole or % weight. Generally, a substantially pure RNA ligase composition will comprise about 60% or more, about 70% or more, about 80% or more, about 90% or more, about 95% or more, and about 98% or more of all macromolecular species by mole or % weight present in the composition. In some embodiments, the object species is purified to essential homogeneity (i.e., contaminant species cannot be detected in the composition by conventional detection methods) wherein the composition consists essentially of a single macromolecular species. Solvent species, small molecules (<500 Daltons), and elemental ion species are not considered macromolecular species. In some embodiments, the isolated recombinant RNA ligase polypeptides are substantially pure polypeptide compositions.

“Improved enzyme property” refers to an engineered RNA ligase polypeptide that exhibits an improvement in any enzyme property as compared to a reference RNA ligase polypeptide, such as a wild-type RNA ligase polypeptide (e.g., the RNA ligase polypeptide sequence of SEQ ID NO: 2 or 14) or another engineered RNA ligase polypeptide. Improved properties include but are not limited to such properties as increased enzymatic activity, increased product yield, increased protein expression, increased thermoactivity, increased thermostability, increased stability, increased substrate specificity and/or affinity, increased substrate range, increased specific activity, increased resistance to substrate and/or end-product inhibition, increased chemical stability, improved solvent stability, increased solubility, and increased inhibitor resistance or tolerance. Exemplary improved properties are provided in the Examples.

“Increased enzymatic activity” and “enhanced catalytic activity” refer to an improved property of the engineered RNA ligase polypeptides, which can be represented by an increase in specific activity (e.g., product produced/time/weight protein) and/or an increase in percent conversion of the substrate to the product (e.g., percent conversion of starting amount of substrate to product in a specified time period using a specified amount of RNA ligase) as compared to the reference RNA ligase enzyme (e.g., wild-type RNA ligase and/or another engineered RNA ligase). Exemplary methods to determine enzyme activity are provided in the Examples. Any property relating to enzyme activity may be affected, including the classical enzyme properties of Kₘ, Vₘₐₓ or k_{cat}, changes of which can lead to increased enzymatic activity. Improvements in enzyme activity can be from about 1.1 fold the enzymatic activity of the corresponding wild-type enzyme, to about 1.5 fold, 2-fold, 5-fold, 10-fold, 20-fold, 25-fold, 50-fold, 75-fold, 100-fold, 150-fold, 200-fold or more enzymatic activity than the naturally occurring RNA ligase or another engineered RNA ligase from which the RNA ligase polypeptides were derived.

“Hybridization stringency” relates to hybridization conditions, such as washing conditions, in the hybridization of nucleic acids. Generally, hybridization reactions are performed under conditions of lower stringency, followed by washes of varying but higher stringency (see, e.g., Sambrook et al., Molecular Cloning: A Laboratory Manual, Cold Spring Harbor Laboratory Press, New York, 2001; Ausubel et al., Current Protocols in Molecular Biology, John Wiley & Sons, 2003). The term “moderately stringent hybridization” refers to conditions that permit target-DNA to bind a complementary nucleic acid that has about 60% identity, preferably about 75% identity, about 85% identity to the target DNA, with greater than about 90% identity to target-polynucleotide. Exemplary moderately stringent conditions are conditions equivalent to hybridization in 50% formamide, 5×Denhart's solution, 5×SSPE, 0.2% SDS at 42° C., followed by washing in 0.2×SSPE, 0.2% SDS, at 42° C. “High stringency hybridization” refers generally to conditions that are about 10° C. or less from the thermal melting temperature Tₘ as determined under the solution condition for a defined polynucleotide sequence. In some embodiments, a high stringency condition refers to conditions that permit hybridization of only those nucleic acid sequences that form stable hybrids in 0.018M NaCl at 65° C. (i.e., if a hybrid is not stable in 0.018M NaCl at 65° C., it will not be stable under high stringency conditions, as contemplated herein). High stringency conditions can be provided, for example, by hybridization in conditions equivalent to 50% formamide, 5×Denhart's solution, 5×SSPE, 0.2% SDS at 42° C., followed by washing in 0.1×SSPE, and 0.1% SDS at 65° C. Another high stringency condition comprises hybridizing in conditions equivalent to hybridizing in 5×SSC containing 0.1% (w:v) SDS at 65° C. and washing in 0.1×SSC containing 0.1% SDS at 65° C. Other high stringency hybridization conditions, as well as moderately stringent conditions, are described in the references cited above.

“Codon optimized” refers to changes in the codons of the polynucleotide encoding a protein to those preferentially used in a particular organism such that the encoded protein is more efficiently expressed in that organism. Although the genetic code is degenerate, in that most amino acids are represented by several codons, called “synonyms” or “synonymous” codons, it is well known that codon usage by particular organisms is nonrandom and biased towards particular codon triplets. This codon usage bias may be higher in reference to a given gene, genes of common function or ancestral origin, highly expressed proteins versus low copy number proteins, and the aggregate protein coding regions of an organism's genome. In some embodiments, the polynucleotides encoding the RNA ligase enzymes are codon optimized for optimal production from the host organism selected for expression.

“Control sequence” refers herein to include all components that are necessary or advantageous for the expression of a polynucleotide and/or polypeptide of the present disclosure. Each control sequence may be native or foreign (e.g., heterologous) to the nucleic acid sequence encoding the polypeptide. Such control sequences include, but are not limited to, leaders, polyadenylation sequences, propeptide sequences, promoter sequences, signal peptide sequences, initiation sequences, and transcription terminators. In some embodiments, the control sequences include a promoter, and transcriptional and translational stop signals. In some embodiments, the control sequences are provided with linkers for the purpose of introducing specific restriction sites facilitating ligation of the control sequences with the coding region of the nucleic acid sequence encoding a polypeptide.

“Operably linked” or “operatively linked” refers to a configuration in which a control sequence is appropriately placed (i.e., in a functional relationship) at a position relative to a polynucleotide of interest such that the control sequence directs or regulates the expression of the polynucleotide of interest, and where appropriate, expression of the encoded polypeptide of interest.

“Promoter” or “promoter sequence” refers to a nucleic acid sequence that is recognized by a host cell for expression of a polynucleotide of interest, such as a coding sequence. The promoter sequence contains transcriptional control sequences that mediate the expression of a polynucleotide of interest. The promoter may be any nucleic acid sequence which shows transcriptional activity in the host cell of choice including mutant, truncated, and hybrid promoters, and may be obtained from genes encoding extracellular or intracellular polypeptides either homologous or heterologous to the host cell.

“Suitable reaction conditions” or “suitable conditions” refers to those conditions in the enzymatic conversion reaction solution (e.g., ranges of enzyme loading, substrate loading, temperature, pH, buffers, co-solvents, etc.) under which an RNA ligase polypeptide of the present disclosure is capable of converting a polynucleotide(s) substrate to the desired ligated product polynucleotide. Exemplary “suitable reaction conditions” are provided herein (see, the Examples).

“Product” in the context of an enzymatic conversion process refers to the compound or molecule resulting from the action of the RNA ligase polypeptide on the substrate.

“Culturing” refers to the growing of a population of cells under suitable conditions using any suitable medium (e.g., liquid, gel, or solid).

“Vector” is a recombinant construct for introducing a polynucleotide of interest into a cell. In some embodiments, the vector is an expression vector that is operably linked to a suitable control sequence capable of effecting the expression in a suitable host of the polynucleotide or a polypeptide encoded in the polynucleotide. In some embodiments, an “expression vector” has a promoter sequence operably linked to the polynucleotide (e.g., transgene) to drive expression in a host cell, and in some embodiments, also comprises a transcription terminator sequence.

“Expression” includes any step involved in the production of the polypeptide including, but not limited to, transcription, post-transcriptional modification, translation, and post-translational modification. In some embodiments, the term also encompasses secretion of the polypeptide from a cell.

“Produces” refers to the production of proteins and/or other compounds by cells. It is intended that the term encompass any step involved in the production of polypeptides including, but not limited to, transcription, post-transcriptional modification, translation, and post-translational modification. In some embodiments, the term also encompasses secretion of the polypeptide from a cell.

“Heterologous” or “recombinant” refers to the relationship between two or more nucleic acid or polypeptide sequences (e.g., a promoter sequence, signal peptide, terminator sequence, etc.) that are derived from different sources and are not associated in nature.

“Host cell” and “host strain” refer to suitable hosts for expression vectors comprising a polynucleotide provided herein (e.g., a polynucleotide sequences encoding at least one RNA ligase variant). In some embodiments, the host cells are prokaryotic or eukaryotic cells that have been transformed or transfected with vectors constructed using recombinant DNA techniques, and progeny thereof, as known in the art.

“Analogue” in the context of a polypeptide means a polypeptide more than 70% sequence identity but less than 100% sequence identity (e.g., more than 75%, 78%, 80%, 83%, 85%, 88%, 90%, 91%, 92%, 93%, 94%, 95%, 96%, 97%, 98%, 99% sequence identity) with a reference polypeptide. In some embodiments, analogues include non-naturally occurring amino acid residues including, but not limited, to homoarginine, ornithine and norvaline, as well as naturally occurring amino acids. In some embodiments, analogues also include one or more D-amino acid residues and non-peptide linkages between two or more amino acid residues.

### Engineered RNA Ligase Polypeptides

In one aspect, the present disclosure provides RNA ligases, including engineered RNA ligase polypeptide variants. In some embodiments, the RNA ligase and engineered RNA ligase polypeptide variants are useful for ligating polynucleotide substrates, such as for preparing polynucleotides from short oligonucleotides, and for diagnostic and other purposes. The engineered RNA ligase variants can be used in solution, as well as in immobilized embodiments. In some embodiments, the engineered RNA ligase can be prepared and used as non-fusion polypeptides or as fusion polypeptides.

In some embodiments, the engineered RNA ligase, or a functional fragment thereof, comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 346 of an even-numbered SEQ ID NO. of SEQ ID NOs: 14 and 24-582, or to a reference sequence corresponding to an even-numbered SEQ ID NO. of SEQ ID NOs: 14 and 24-582, wherein the amino acid sequence comprises one or more substitutions relative to a reference sequence corresponding to residues 12 to 346 of SEQ ID NO: 14, 42, or 204, or relative to a reference sequence corresponding to SEQ ID NO: 14, 42, or 204.

In some embodiments, the engineered RNA ligase comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 14, 42, or 204, or to the reference sequence corresponding to SEQ ID NO: 14, 42, or 204, wherein the amino acid sequence comprises one or more substitutions relative to the reference sequence corresponding to residues 12 to 346 of SEQ ID NO: 14, 42, or 204, or relative to the reference sequence corresponding to SEQ ID NO: 14, 42, or 204.

In some embodiments, the engineered RNA ligase comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 14, or to the reference sequence corresponding to SEQ ID NO: 14, wherein the amino acid sequence comprises one or more substitutions relative to the reference sequence corresponding to residues 12 to 346 of SEQ ID NO: 14, or relative to the reference sequence corresponding to SEQ ID NO: 14.

In some embodiments, the engineered RNA ligase comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 42 or 204, or to the reference sequence corresponding to SEQ ID NO: 42 or 204, wherein the amino acid sequence comprises one or more substitutions relative to the reference sequence corresponding to residues 12 to 346 of SEQ ID NO: 14, or relative to the reference sequence corresponding to SEQ ID NO: 14.

In some embodiments, the engineered RNA ligase comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or more sequence identity to the reference sequence corresponding to residues 12 to 346 of an even numbered SEQ ID NO. of SEQ ID NOs: 24-582, or to the reference sequence corresponding to an even numbered SEQ ID NO. of SEQ ID NOs: 24-582, wherein the amino acid sequence comprises one or more substitutions relative to the reference sequence corresponding to residues 12 to 346 of SEQ ID NO: 14, or relative to the reference sequence corresponding to SEQ ID NO: 14.

In some embodiments, the engineered RNA ligase comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 24-128, or to the reference sequence corresponding to an even-numbered SEQ ID NO. of SEQ ID NOs: 24-128, wherein the amino acid sequence comprises one or more substitutions relative to the reference sequence corresponding to residues 12 to 346 of SEQ ID NO: 14, or relative to the reference sequence corresponding to SEQ ID NO: 14.

In some embodiments, the amino acid sequence of the engineered RNA ligase comprises at least a substitution at amino acid position 2, 14, 15, 18, 33, 39, 63, 69, 81, 83, 85, 86, 89, 95, 116, 117, 119, 138, 142, 144, 149, 154, 156, 159, 171, 175, 177, 181, 185, 186, 195, 202, 212, 214, 224, 226, 230, 247, 256, 257, 260, 266, 275, 280, 283, 285, 288, 291, 296, 303, 306, 307, 310, 314, 315, 316, 317, 326, 330, 331, 333, 334, 335, 337, 339, 342, or 345, or combinations thereof, wherein the amino acid positions are relative to the reference sequence corresponding to residues 12 to 346 of SEQ ID NO: 14, or relative to the reference sequence corresponding to SEQ ID NO: 14.

In some embodiments, the amino acid sequence of the engineered RNA ligase comprises at least a substitution 2G/V/E, 14K, 15L/E/M/P/Y, 18I, 33L, 39Q/S, 63V, 69D, 81Y, 85L, 86G, 89H/I/M/W, 95V, 116N, 117D, 119T, 138R, 142R, 144W, 149R/T, 154C, 156A/L/N/Q/T, 159F, 171E, 175K, 177P, 181D, 183V, 185G/K, 186D/R, 195G, 202Y, 212I, 214I, 224Y, 226I, 230D, 247K, 256A, 257L, 260S, 266L, 275N, 280G/N, 283I, 285K, 288E, 291P, 296K, 303Q, 306L, 307E/Q, 310K, 314W, 315S/T, 316L, 317A, 326R, 330M/R, 331R/W, 333V, 334R, 335D/H, 337L, 339P, 342I, or 345V, or combinations thereof, wherein the amino acid positions are relative to the reference sequence corresponding to residues 12 to 346 of SEQ ID NO: 14, or relative to the reference sequence corresponding to SEQ ID NO: 14.

In some embodiments, the amino acid sequence of the engineered RNA ligase comprises at least a substitution H2G/V/E, A14K, K15L/E/M/P/Y, S18I, G33L, H39Q/S, I63V, T69D, H81Y, M85L, K86G, K89H/I/M/W, M95V, G116N, G117D, Q119T, S138R, K142R, Y144W, V149R/T, A154C, G156A/L/N/Q/T, L159F, A171E, Q175K, V177P, K181D, I183V, N185G/K, Y186D/R, D195G, H202Y, T212I, V214I, N224Y, K226I, N230D, A247K, K256A, V257L, T260S, V266L, T275N, S280G/N, L283I, H285K, S288E, A291P, R296K, K303Q, I306L, N307E/Q, A310K, H314W, D315S/T, M316L, L317A, K326R, T330M/R, I331R/W, I333V, Q334R, N335D/H, I337L, S339P, H342I, or L345V, or combinations thereof, wherein the amino acid positions are relative to the reference sequence corresponding to residues 12 to 346 of SEQ ID NO: 14, or relative to the reference sequence corresponding to SEQ ID NO: 14.

In some embodiments, the amino acid sequence of the engineered RNA ligase comprises at least a substitution at amino acid position 95 or 177, or combination thereof, wherein the amino acid positions are relative to the reference sequence corresponding to residues 12 to 346 of SEQ ID NO: 14, or relative to the reference sequence corresponding to SEQ ID NO: 14.

In some embodiments, the amino acid sequence of the engineered RNA ligase comprises at least a substitution or amino acid residue 95V or 177P, or combination thereof, wherein the amino acid positions are relative to the reference sequence corresponding to residues 12 to 346 of SEQ ID NO: 14, or relative to the reference sequence corresponding to SEQ ID NO: 14.

In some embodiments, the amino acid sequence of the engineered RNA ligase comprises at least a substitution M95V or V177P, or combination thereof, wherein the amino acid positions are relative to the reference sequence corresponding to residues 12 to 346 of SEQ ID NO: 14, or relative to the reference sequence corresponding to SEQ ID NO: 14.

In some embodiments, the amino acid sequence of the engineered RNA ligase comprises at least a substitution at amino acid position 15, 149, 154, 156, 186, 195, 224, or 226, or combinations thereof, wherein the amino acid positions are relative to the reference sequence corresponding to residues 12 to 346 of SEQ ID NO: 14, or relative to the reference sequence corresponding to SEQ ID NO: 14.

In some embodiments, the amino acid sequence of the engineered RNA ligase comprises at least a substitution or amino acid residue 15Y, 149R, 154C, 156T, 186R, 195G, 224Y, or 226I, or combinations thereof, wherein the amino acid positions are relative to the reference sequence corresponding to residues 12 to 346 of SEQ ID NO: 14, or relative to the reference sequence corresponding to SEQ ID NO: 14.

In some embodiments, the amino acid sequence of the engineered RNA ligase comprises at least a substitution K15Y, V149R/T, A154C, G156A/L/N/Q/T, Y186D/R, D195G, N224Y, or K226I, or combinations thereof, wherein the amino acid positions are relative to the reference sequence corresponding to residues 12 to 346 of SEQ ID NO: 14, or relative to the reference sequence corresponding to SEQ ID NO: 14.

In some embodiments, the amino acid sequence of the engineered RNA ligase comprises at least a substitution at amino acid position 15, 95, 149, 154, 156, 177, 186, 195, 224, or 226, or combinations thereof, wherein the amino acid positions are relative to the reference sequence corresponding to residues 12 to 346 of SEQ ID NO: 14, or relative to the reference sequence corresponding to SEQ ID NO: 14.

In some embodiments, the amino acid sequence of the engineered RNA ligase comprises at least a substitution or amino acid residue 15L/E/M/P/Y, 95V, 149R/T, 154C, 156A/L/N/Q/T, 177P, 186D/R, 195G, 224Y, or 226I, or combinations thereof, wherein the amino acid positions are relative to the reference sequence corresponding to residues 12 to 346 of SEQ ID NO: 14, or relative to the reference sequence corresponding to SEQ ID NO: 14.

In some embodiments, the amino acid sequence of the engineered RNA ligase comprises at least a substitution or amino acid residue 15Y, 95V, 149R, 154C, 156T, 177P, 186R, 195G, 224Y, or 226I, or combinations thereof, wherein the amino acid positions are relative to the reference sequence corresponding to residues 12 to 346 of SEQ ID NO: 14, or relative to the reference sequence corresponding to SEQ ID NO: 14.

In some embodiments, the amino acid sequence of the engineered RNA ligase comprises at least a substitution K15Y, M95V, V149R, A154C, G156T, V177P, Y186R, D195G, N224Y, or K226I, or combinations thereof, wherein the amino acid positions are relative to the reference sequence corresponding to residues 12 to 346 of SEQ ID NO: 14, or relative to the reference sequence corresponding to SEQ ID NO: 14.

In some embodiments, the amino acid sequence of the engineered RNA ligase comprises at least a substitution or substitution set at amino acid position(s) 177/260/307/345, 171/175/183/280/283/303, 95/159/177/260/288, 175/183/247/280/283/296, 177/307, 81/171/183/335, 175/183, 81/175/183/280/296/335/339, 81/183/247/266, 95/177, 171/280/283/296/303, 85/177/260/288/333/345, 177/291/307/333, 81/171/175/183/316/337, 81/171/266/280/283/335/339, 177, 95/159/177/260/280/288/306/345, 171/175/335, 177/260/30/333, 14/95/177/288/307, 95/177/345, 95/159/177/260/285/288, 266/296, 81/283/335/337, 95/159/177/260/342/345, 63/171/175/183/266/280/296/316, 280/335/337, 81/171/280, 81/171/175/296/316, 171/175/316/335, 171/280/283/296/316, 171/283/335, 63/171/175/183/266/283/303/316, 171/247/266/283/296/303/316/335, 63/81/171/175/183/266/280/283/337, 81/171/303/316/335, 288/307, 14/95/159/177/345, 81/171/175/183/247/266/280/283, 81/247/266, 280, 85/177/260, 63/183, 175/183/247/280/283/316, 291, 247, 171/280/283, 81/171/175/280/316, 333, 171/296/316, 81/171, or 280/316/335, wherein the amino acid positions are relative to the reference sequence corresponding to residues 12 to 346 of SEQ ID NO: 14, or relative to the reference sequence corresponding to SEQ ID NO: 14.

In some embodiments, the amino acid sequence of the engineered RNA ligase comprises at least a substitution or substitution set 177P/260S/307E/345V, 171E/175K/183V/280N/283I/303Q, 95V/159F/177P/260S/288E, 175K/183V/247K/280N/283I/296K, 177P/307E, 81Y/171E/183V/335D, 175K/183V, 81Y/175K/183V/280N/296K/335D/339P, 81Y/183V/247K/266L, 95V/177P, 171E/280N/283I/296K/303Q, 85L/177P/260S/288E/333V/345V, 177P/291P/307E/333V, 81Y/171E/175K/183V/316L/337L, 81Y/171E/266L/280N/283I/335D/339P, 177P, 95V/159F/177P/260S/280N/288E/306L/345V, 171E/175K/335D, 177P/260S/306L/333V, 14K/95V/177P/288E/307E, 95V/177P/345V, 95V/159F/177P/260S/285K/288E, 266L/296K, 81Y/283I/335D/337L, 95V/159F/177P/260S/342I/345V, 63V/171E/175K/183V/266L/280N/296K/316L, 280N/335D/337L, 81Y/171E/280N, 81Y/171E/175K/296K/316L, 171E/175K/316L/335D, 171E/280N/283I/296K/316L, 171E/283I/335D, 63V/171E/175K/183V/266L/283I/303Q/316L, 171E/247K/266L/283I/296K/303Q/316L/335D, 63V/81Y/171E/175K/183V/266L/280N/283I/337L, 81Y/171E/303Q/316L/335D, 288E/307E, 14K/95V/159F/177P/345V, 81Y/171E/175K/183V/247K/266L/280N/283I, 81Y/247K/266L, 280N, 85L/177P/260S, 63V/183V, 175K/183V/247K/280N/283I/316L, 291P, 247K, 171E/280N/283I, 81Y/171E/175K/280N/316L, 333V, 171E/296K/316L, 81Y/171E, or 280N/316L/335D, wherein the amino acid positions are relative to the reference sequence corresponding to residues 12 to 346 of SEQ ID NO: 14, or relative to the reference sequence corresponding to SEQ ID NO: 14.

In some embodiments, the amino acid sequence of the engineered RNA ligase comprises at least a substitution or substitution set V177P/T260S/N307E/L345V, A171E/Q175K/I183V/S280N/L283I/K303Q, M95V/L159F/V177P/T260S/S288E, Q175K/I183V/A247K/S280N/L283I/R296K, V177P/N307E, H81Y/A171E/I183V/N335D, Q175K/I183V, H81Y/Q175K/I183V/S280N/R296K/N335D/S339P, H81Y/I183V/A247K/V266L, M95V/V177P, A171E/S280N/L283I/R296K/K303Q, M85L/V177P/T260S/S288E/I333V/L345V, V177P/A291P/N307E/I333V, H81Y/A171E/Q175K/I183V/M316L/I337L, H81Y/A171E/V266L/S280N/L283I/N335D/S339P, V177P, M95V/L159F/V177P/T260S/S280N/S288E/I306L/L345V, A171E/Q175K/N335D, V177P/T260S/I306L/I333V, A14K/M95V/V177P/S288E/N307E, M95V/V177P/L345V, M95V/L159F/V177P/T260S/H285K/S288E, V266L/R296K, H81Y/L283I/N335D/I337L, M95V/L159F/V177P/T260S/H342I/L345V, I63V/A171E/Q175K/I183V/V266L/S280N/R296K/M316L, S280N/N335D/I337L, H81Y/A171E/S280N, H81Y/A171E/Q175K/R296K/M316L, A171E/Q175K/M316L/N335D, A171E/S280N/L283I/R296K/M316L, A171E/L283I/N335D, I63V/A171E/Q175K/I183V/V266L/L283I/K303Q/M316L, A171E/A247K/V266L/L283I/R296K/K303Q/M316L/N335D, I63V/H81Y/A171E/Q175K/I183V/V266L/S280N/L283/1337L, H81Y/A171E/K303Q/M316L/N335D, S288E/N307E, A14K/M95V/L159F/V177P/L345V, H81Y/A171E/Q175K/I183V/A247K/V266L/S280N/L283I, H81Y/A247K/V266L, S280N, M85L/V177P/T260S, I63V/I183V, Q175K/I183V/A247K/S280N/L283I/M316L, A291P, A247K, A171E/S280N/L283I, H81Y/A171E/Q175K/S280N/M316L, I333V, A171E/R296K/M316L, H81Y/A171E, or S280N/M316L/N335D, wherein the amino acid positions are relative to the reference sequence corresponding to residues 12 to 346 of SEQ ID NO: 14, or relative to the reference sequence corresponding to SEQ ID NO: 14.

In some embodiments, the amino acid sequence of the engineered RNA ligase comprises at least a substitution set at amino acid positions 95/149/156/177/195, 15/95/149/177/186/224/317, 15/95/156/177/195/224/226/317/331, 15/95/177/195/317/331, 95/149/177/224/331, 15/95/177/195/224/226/317/331, 15/33/86/95/149/154/177/195/317, 15/86/95/154/156/177/186/195/224/226/331, 15/95/149/177/224/317/331, 15/95/154/156/177/186/317/331, 15/33/95/149/177/224/226/331, 15/95/177/186/224/226/317/331, 15/33/95/149/177/186/224/331, 95/177/195/224/331, 15/86/95/156/177/186/195/226/317/331, 15/95/177/195/226/331, 95/156/177/186/224/226/331, 15/95/149/154/156/177/224/226, 33/95/156/177/186/195/331, 15/86/95/149/154/156/177/317/331, 15/95/177/186/317/331, 15/86/95/149/154/177/195/331, 15/33/95/149/154/156/177/226, 15/95/149/154/177/186/317/331, 95/177/195/331, 15/86/95/156/177/186/195/331, 95/149/156/177/224/226/331, 15/95/149/154/156/177/186/317/331, 15/95/154/177/186/195, 15/86/95/177/195/224/226, 95/177/230, 15/33/95/156/177/331, 15/33/95/156/177/195/224/226, 33/95/149/156/177/317, 15/33/95/177/186/195, 95/177/334, 15/95/154/156/177/224/317, 15/95/149/177/186/331, 15/86/95/149/177/186/195/317, 15/33/95/154/177/195, 15/95/156/177/186/331, 15/95/149/177/186/317, 15/33/95/156/177/186/214/224/331, 15/86/95/177/186/224/226/317, 15/95/154/177/331, 15/95/149/154/177/226, 15/33/95/177/195/226, 15/95/177/224/317/331, 15/86/95/177/186/195/317, 95/177/226/317/331, 95/177/330, 89/95/177, 15/95/149/177/186, 95/156/177/195/331, 95/156/177, 15/86/95/156/177/186/331, 95/177/275, 15/33/95/177/186/224, 15/95/177/186/317, 15/33/95/177/186/226/317, 15/95/149/154/156/177/331, 15/95/154/177/226/317, 15/86/95/156/177/186/195/317, 95/177/257, 15/95/154/156/177/186, 33/95/177/186/224, 95/177/335, 15/86/95/156/177/186/195/224/331, 95/154/156/177/317, 39/95/177, 2/95/177, 15/95/177/224/226/331, 15/33/95/149/177/224, 89/95/177/307, 15/95/154/156/177/317, 15/95/177/186/331, 15/33/95/154/177/317, 15/33/95/177/195/224/317, 15/95/156/177/224/226, 95/154/177/186/331, 95/177/185, 15/18/95/149/177/186/195, 86/95/149/154/156/177, 15/33/95/149/177/331, 95/177/326, 95/177/202, 95/119/177, 95/177/212, 15/33/95/156/177/317, 95/177/280, 95/142/177, 15/95/149/177, 15/33/86/95/156/177/195, 95/177/315, 95/177/256, 15/33/95/156/177/186, 95/138/177, 95/117/177, 95/116/177, 95/177/316, 69/95/177, 95/177/310, 95/177/317, 15/95/177/195/331, 15/33/95/177/331, 95/177/314, or 95/144/177, wherein the amino acid positions are relative to the reference sequence corresponding to residues 12 to 346 of SEQ ID NO: 14, or relative to the reference sequence corresponding to SEQ ID NO: 14.

In some embodiments, the amino acid sequence of the engineered RNA ligase comprises at least a substitution set 95V/149R/156T/177P/195G, 15M/95V/149T/177P/186R/224Y/317A, 15P/95V/156T/177P/195G/224Y/226I/317A/331L, 15M/95V/177P/195G/317A/331R, 95V/149R/177P/224Y/331R, 15E/95V/177P/195G/224Y/226I/317A/331W, 15P/33L/86G/95V/149R/154C/177P/195G/317A, 15M/86G/95V/154C/156T/177P/186R/195G/224Y/226I/331L, 15P/95V/149R/177P/224Y/317A/331R, 15M/95V/154C/156T/177P/186R/317A/331R, 15M/33L/95V/149R/177P/224Y/226I/331W, 15E/95V/177P/186R/224Y/226I/317A/331R, 15Y/33L/95V/149R/177P/186R/224Y/331R, 95V/177P/195G/224Y/331R, 15P/86G/95V/156T/177P/186R/195G/226I/317A/331L, 15M/95V/177P/195G/226I/331W, 95V/156T/177P/186R/224Y/226I/331R, 15Y/95V/149R/154C/156T/177P/224Y/226I, 33L/95V/156T/177P/186R/195G/331L, 15P/86G/95V/149R/154C/156T/177P/317A/331R, 15M/95V/177P/186D/317A/331R, 15P/86G/95V/149R/154C/177P/195G/331L, 15Y/33L/95V/149T/154C/156T/177P/226I, 15P/95V/149R/154C/177P/186R/317A/331L, 95V/177P/195G/331R, 15M/86G/95V/156T/177P/186R/195G/331W, 95V/149R/156T/177P/224Y/226I/331W, 15P/95V/149R/154C/156T/177P/186R/317A/331L, 15Y/95V/154C/177P/186D/195G, 15E/86G/95V/177P/195G/224Y/226I, 95V/177P/230D, 15P/33L/95V/156T/177P/331R, 15L/33L/95V/156T/177P/195G/224Y/226I, 33L/95V/149R/156T/177P/317A, 15E/33L/95V/177P/186R/195G, 95V/177P/334R, 15E/95V/154C/156T/177P/224Y/317A, 15Y/95V/149R/177P/186R/331W, 15Y/86G/95V/149T/177P/186R/195G/317A, 15P/33L/95V/154C/177P/195G, 15P/95V/154C/156T/177P/186R/317A/331R, 15M/95V/156T/177P/186R/331R, 15Y/95V/149T/177P/186D/317A, 15E/33L/95V/156T/177P/186R/214I/224Y/331W, 15E/86G/95V/177P/186R/224Y/226I/317A, 15P/95V/154C/177P/331R, 15Y/95V/149T/154C/177P/226I, 15Y/33L/95V/177P/195G/226I, 15M/95V/177P/224Y/317A/331W, 15E/86G/95V/177P/186R/195G/317A, 95V/177P/226I/317A/331L, 95V/177P/330R, 89W/95V/177P, 15P/95V/149R/177P/186D/331L, 15P/95V/149R/177P/186R, 89M/95V/177P, 95V/156T/177P/195G/331L, 95V/156A/177P, 15E/86G/95V/156T/177P/186R/331R, 95V/177P/195G/331W, 95V/177P/275N, 15P/33L/95V/177P/186R/224Y, 15E/95V/177P/186R/317A, 15M/33L/95V/177P/186R/226I/317A, 89G/95V/177P, 15Y/95V/149R/154C/156T/177P/331W, 15Y/95V/154C/177P/226I/317A, 15Y/86G/95V/156T/177P/186R/195G/317A, 95V/177P/257L, 15Y/95V/154C/156T/177P/186D, 33L/95V/177P/186R/224Y, 95V/177P/335H, 15Y/86G/95V/156T/177P/186R/195G/224Y/331W, 95V/154C/156T/177P/317A, 39Q/95V/177P, 2G/95V/177P, 15M/95V/177P/224Y/226I/331R, 15M/33L/95V/149R/177P/224Y, 89I/95V/177P/307Q, 15M/95V/154C/156T/177P/317A, 15E/95V/177P/186R/331W, 15P/33L/95V/154C/177P/317A, 89H/95V/177P, 15Y/33L/95V/177P/195G/224Y/317A, 15E/95V/156T/177P/224Y/226I, 95V/154C/177P/186R/331L, 95V/177P/185G, 15M/18I/95V/149R/177P/186R/195G, 86G/95V/149R/154C/156T/177P, 15P/33L/95V/149R/177P/331L, 95V/177P/326R, 95V/177P/202Y, 95V/119T/177P, 95V/177P/212I, 2V/95V/177P, 15E/95V/149R/154C/156T/177P/331L, 15P/33L/95V/156T/177P/317A, 15Y/95V/154C/156T/177P/317A, 95V/156L/177P, 95V/177P/335D, 95V/156T/177P, 2E/95V/177P, 95V/177P/280G, 95V/142R/177P, 15E/95V/149R/177P, 95V/156Q/177P, 15E/33L/86G/95V/156T/177P/195G, 95V/177P/315T, 95V/177P/185K, 39S/95V/177P, 95V/177P/256A, 15Y/33L/95V/156T/177P/186D, 95V/138R/177P, 95V/117D/177P, 95V/116N/177P, 95V/177P/316L, 15M/95V/149R/177P, 69D/95V/177P, 95V/156N/177P, 95V/177P/310K, 95V/177P/317A, 15Y/95V/177P/195G/331L, 15M/33L/95V/177P/331L, 95V/177P/330M, 95V/177P/314W, 95V/144W/177P, 15Y/95V/149R/177P, or 95V/177P/315S, wherein the amino acid positions are relative to the reference sequence corresponding to residues 12 to 346 of SEQ ID NO: 14, or relative to the reference sequence corresponding to SEQ ID NO: 14.

In some embodiments, the amino acid sequence of the engineered RNA ligase comprises at least a substitution or substitution set provided in Table 6.1, wherein the amino acid positions are relative to the reference sequence corresponding to residues 12 to 346 of SEQ ID NO: 14, or relative to the reference sequence corresponding to SEQ ID NO: 14.

In some embodiments, the amino acid sequence of the engineered RNA ligase comprises at least a substitution set at amino acid positions 15/95/149/154/156/177/186/195/331, 95/149/154/177/186/195/224/317/331, 15/33/95/149/154/177/186/195, 95/149/156/177/186/195/224/226/317, 15/95/149/156/177/195/224/226, 15/33/86/95/149/154/156/177/186/195/226/331, 15/95/149/156/177/186/195/224/317/331, 15/33/95/149/154/177/195/317, 15/95/149/154/177/195/224/317, 15/95/149/156/177/186/224/226/317/331, 15/33/95/149/154/156/177/186/195/224/226/317/331, 95/149/177/195/224, 86/95/149/154/156/177/186/195/331, 15/33/95/149/156/177/195/224/317/331, 15/95/149/154/156/177/186/195/317/331, 15/33/95/149/154/156/177/186/224/331, 15/95/149/154/156/177/195/224, 15/95/149/154/156/177/195/224/226, 15/95/149/154/156/177/186/195/224/317, 33/95/149/156/177/186/224/331, 15/33/86/95/149/177/195/224/331, 15/86/95/149/154/156/177/186/195/331, 95/149/177/186/195/331, 15/95/154/156/177/186/226/331, 15/95/149/154/177/186/195/224/226, 15/95/149/154/156/177/195/224/331, 15/33/95/149/154/156/177/195/317/331, 15/33/95/154/156/177/186/195/224/317/331, 15/95/149/177/186/195/224/331, 15/33/95/149/154/156/177/195/224/331, 15/95/149/154/156/177/224/226/317/331, 15/33/95/149/156/177/195/331, 15/95/149/154/156/177/186/224/226/331, 95/154/156/177/195/224/226, 15/33/86/95/149/154/156/177/186/195/224/317/331, 15/95/149/154/156/177/186/195/224/226, 15/33/95/149/177/195/317/331, 15/95/149/154/177/224/331, 95/149/154/156/177/195/224/226/331, 15/95/149/154/177/195/331, 15/95/149/154/156/177/186/195, 15/33/95/149/154/156/177/186/195, 15/33/86/95/149/154/177/195/224/317/331, or 95/149/177/186/195/224/226/331, wherein the amino acid positions are relative to the reference sequence corresponding to residues 12 to 346 of SEQ ID NO: 14, or relative to the reference sequence corresponding to SEQ ID NO: 14.

In some embodiments, the amino acid sequence of the engineered RNA ligase comprises at least a substitution set 15M/95V/149R/154C/156T/177P/186R/195G/331R, 95V/149R/154C/177P/186D/195G/224Y/317A/331R, 15M/33L/95V/149T/154C/177P/186R/195G, 95V/149R/156T/177P/186R/195G/224Y/226I/317A, 15Y/95V/149R/156T/177P/195G/224Y/226I, 15Y/33L/86G/95V/149T/154C/156T/177P/186R/195G/226I/331W, 15Y/95V/149R/156T/177P/186R/195G/224Y/317A/331W, 15M/33L/95V/149T/154C/177P/195G/317A, 15M/95V/149R/154C/177P/195G/224Y/317A, 15Y/95V/149R/156T/177P/186D/224Y/226I/317A/331R, 15M/95V/149T/154C/156T/177P/186D/195G/331L, 15Y/33L/95V/149R/154C/156T/177P/186R/195G/224Y/226I/317A/331R, 95V/149R/177P/195G/224Y, 86G/95V/149T/154C/156T/177P/186R/195G/331L, 15P/33L/95V/149R/156T/177P/195G/224Y/317A/331L, 15P/95V/149T/154C/156T/177P/186D/195G/317A/331L, 15Y/33L/95V/149T/154C/156T/177P/186D/224Y/331L, 15P/95V/149R/156T/177P/186R/195G/224Y/317A/331W, 15Y/95V/149R/154C/156T/177P/195G/224Y, 15M/95V/149T/154C/156T/177P/195G/224Y/226I, 15E/95V/149R/154C/156T/177P/186R/195G/224Y/317A, 33L/95V/149T/156T/177P/186R/224Y/331L, 15Y/33L/86G/95V/149R/177P/195G/224Y/331W, 15E/86G/95V/149R/154C/156T/177P/186R/195G/331W, 95V/149R/177P/186R/195G/331W, 15E/95V/154C/156T/177P/186D/226I/331R, 15Y/95V/149T/154C/177P/186R/195G/224Y/226I, 15P/95V/149R/154C/156T/177P/195G/224Y/331W, 15E/33L/95V/149R/154C/156T/177P/195G/317A/331L, 15E/33L/95V/154C/156T/177P/186R/195G/224Y/317A/331L, 15P/95V/149R/177P/186D/195G/224Y/331L, 15P/33L/95V/149R/154C/156T/177P/195G/224Y/331W, 15P/95V/149T/154C/156T/177P/224Y/226I/317A/331W, 15P/33L/95V/149T/156T/177P/195G/331W, 15P/95V/149T/154C/156T/177P/186D/224Y/226I/331L, 95V/154C/156T/177P/195G/224Y/226I, 15Y/33L/86G/95V/149R/154C/156T/177P/186D/195G/224Y/317A/331W, 15Y/95V/149R/154C/156T/177P/186R/195G/224Y/226I, 15P/33L/95V/149R/177P/195G/317A/331L, 15E/95V/149T/154C/177P/224Y/331R, 95V/149T/154C/156T/177P/195G/224Y/226I/331R, 15M/95V/149R/154C/177P/195G/331L, 15Y/95V/149R/154C/156T/177P/186R/195G, 15E/95V/149T/154C/156T/177P/186R/195G/331R, 15M/33L/95V/149T/154C/156T/177P/186R/195G, 15P/33L/86G/95V/149R/154C/177P/195G/224Y/317A/331R, or 95V/149R/177P/186D/195G/224Y/226I/331R, wherein the amino acid positions are relative to the reference sequence corresponding to residues 12 to 346 of SEQ ID NO: 14, or relative to the reference sequence corresponding to SEQ ID NO: 14.

In some embodiments, the amino acid sequence of the engineered RNA ligase comprises at least a substitution or substitution set provided in Tables 7.1, 8.1, and 9.1, wherein the amino acid positions are relative to the reference sequence corresponding to residues 12 to 346 of SEQ ID NO: 14, or relative to the reference sequence corresponding to SEQ ID NO: 14.

In some embodiments, the amino acid sequence of the engineered RNA ligase comprises at least a substitution set at amino acid positions 2/15/39/69/95/144/149/154/156/177/186/195/224/226/316/330, 2/15/39/69/95/119/149/154/156/177/185/186/195/224/226/316, 15/69/95/149/154/156/177/186/195/212/224/226/256/330, 15/39/95/119/149/154/156/177/186/195/224/226/256/316/326/330, 15/69/95/116/119/149/154/156/177/185/186/195/224/226/330, 2/15/39/95/119/144/149/154/156/159/177/186/195/202/224/226/256/316/326/330, 15/81/95/149/154/156/177/183/185/186/195/224/226/230, 2/15/39/69/95/116/144/149/154/156/177/185/186/195/224/226, 2/14/15/39/69/95/116/119/149/154/156/159/177/185/186/195/224/226/256/326, 2/15/95/116/119/149/154/156/177/186/195/224/226/316, 15/95/116/119/144/149/154/156/177/185/186/195/224/226/314/316, 15/39/69/95/116/144/149/154/156/177/181/185/186/195/224/226/256/330, 2/15/39/69/95/119/144/149/154/156/177/185/186/195/224/226/314, 14/15/39/69/95/144/149/154/156/177/186/195/212/224/226/256/314/316/326/330, 15/39/69/95/144/149/154/156/177/185/186/195/224/226/316/330, 15/95/149/154/156/177/185/186/195/224/226/334/335, 15/39/63/95/149/154/156/177/186/195/224/226/230/257/275/330/335, 15/95/149/154/156/177/186/195/224/226/266/316/330/334/335, 15/39/63/95/149/154/156/177/183/185/186/195/224/226/230/330/334, 15/69/95/144/149/154/156/177/186/195/224/226/316, 15/95/116/119/149/154/156/177/186/195/202/212/224/226/326/330, 15/39/69/95/119/138/149/154/156/177/181/185/186/195/224/226/316/326, 15/39/81/95/149/154/156/177/186/195/224/226/230, 2/14/15/69/95/116/119/144/149/154/156/159/177/186/195/224/226/326/330, 15/39/95/149/154/156/177/186/195/224/226/334/345, 15/95/149/154/156/177/186/195/224/226/230/266/334, 2/15/95/116/144/149/154/156/159/177/186/195/224/226, 15/95/149/154/156/177/183/185/186/195/224/226/230, 2/14/15/95/116/149/154/156/177/186/195/224/226/316/326/330, 15/39/63/95/149/154/156/177/186/195/224/226/230/316/345, 15/69/95/138/144/149/154/156/159/177/185/186/195/202/224/226/316, 15/39/95/149/154/156/177/186/195/224/226/257/266/316/334, 15/95/149/154/156/177/186/195/224/226/257/275/334, 15/95/149/154/156/177/183/186/195/224/226, 15/89/95/149/154/156/177/185/186/195/224/226/230/316/345, 2/14/15/95/119/149/154/156/177/186/195/224/226, 15/95/144/149/154/156/177/186/195/224/226/316/330, 15/39/81/95/149/154/156/177/185/186/195/224/226/316/334, 15/89/95/149/154/156/177/186/195/224/226/230/266/345, 15/39/95/149/154/156/177/186/195/224/226/230/275/316, 15/39/95/149/154/156/177/186/195/224/226/230/266/334/335, 15/95/149/154/156/177/185/186/195/224/226/275/316/330, 15/95/119/149/154/156/177/186/195/202/224/226, 15/95/149/154/156/177/186/195/224/226/230/257/275/316/330/345, 15/95/149/154/156/177/186/195/224/226/230, 15/81/95/149/154/156/177/186/195/224/226/330/335/345, 15/95/149/154/156/177/183/186/195/224/226/230/266, 15/39/95/149/154/156/177/185/186/195/224/226, 15/39/81/95/149/154/156/177/183/186/195/224/226/275/316/334/345, 15/95/149/154/156/177/186/195/224/226/230/257/316, 15/39/95/116/149/154/156/177/186/195/224/226, or 15/95/149/154/156/177/183/185/186/195/224/226/257/275/316/330/334, wherein the amino acid positions are relative to the reference sequence corresponding to residues 12 to 346 of SEQ ID NO: 14, or relative to the reference sequence corresponding to SEQ ID NO: 14.

In some embodiments, the amino acid sequence of the engineered RNA ligase comprises at least a substitution set 2E/15Y/39S/69D/95V/144W/149R/154C/156L/177P/186R/195G/224Y/226I/316L/330M, 2V/15Y/39S/69D/95V/119T/149R/154C/156T/177P/185K/186R/195G/224Y/226I/316L, 15Y/69D/95V/149R/154C/156T/177P/186R/195G/212I/224Y/226I/256A/330M, 15Y/39S/95V/119T/149R/154C/156T/177P/186R/195G/224Y/226I/256A/316L/326R/330M, 15Y/69D/95V/116N/119T/149R/154C/156T/177P/185K/186R/195G/224Y/226I/330M, 2E/15Y/39S/95V/119T/144W/149R/154C/156N/159F/177P/186R/195G/202Y/224Y/226I/256A/316L/326R/330M, 15Y/81Y/95V/149R/154C/156T/177P/183V/185G/186R/195G/224Y/226I/230D, 2V/15Y/39S/69D/95V/116N/144W/149R/154C/156L/177P/185K/186R/195G/224Y/226I, 2E/14K/15Y/39S/69D/95V/116N/119T/149R/154C/156T/159F/177P/185K/186R/195G/224Y/226I/256A/326R, 2E/15Y/95V/116N/119T/149R/154C/156T/177P/186R/195G/224Y/226I/316L, 15Y/95V/116N/119T/144W/149R/154C/156T/177P/185K/186R/195G/224Y/226I/314W/316L, 15Y/39S/69D/95V/116N/144W/149R/154C/156T/177P/181D/185K/186R/195G/224Y/226I/256A/330M, 2E/15Y/39S/69D/95V/119T/144W/149R/154C/156L/177P/185K/186R/195G/224Y/226I/314W, 14K/15Y/39S/69D/95V/144W/149R/154C/156N/177P/186R/195G/212I/224Y/226I/256A/314W/316L/326R/330M, 15Y/39S/69D/95V/144W/149R/154C/156L/177P/185K/186R/195G/224Y/226I/316L/330M, 15Y/95V/149R/154C/156Q/177P/185G/186R/195G/224Y/226I/334R/335H, 15Y/39Q/63V/95V/149R/154C/156Q/177P/186R/195G/224Y/226I/230D/257L/275N/330R/335H, 15Y/95V/149R/154C/156Q/177P/186R/195G/224Y/226I/266L/316L/330R/334R/335H, 15Y/39Q/63V/95V/149R/154C/156Q/177P/183V/185G/186R/195G/224Y/226I/230D/330R/334R, 15Y/69D/95V/144W/149R/154C/156T/177P/186R/195G/224Y/226I/316L, 15Y/95V/116N/119T/149R/154C/156L/177P/186R/195G/202Y/212I/224Y/226I/326R/330M, 15Y/39S/69D/95V/119T/138R/149R/154C/156T/177P/181D/185K/186R/195G/224Y/226I/316L/326R, 15Y/39Q/81Y/95V/149R/154C/156Q/177P/186R/195G/224Y/226I/230D, 2V/14K/15Y/69D/95V/116N/119T/144W/149R/154C/156T/159F/177P/186R/195G/224Y/226I/326R/330M, 15Y/39Q/95V/149R/154C/156A/177P/186R/195G/224Y/226I/334R/345V, 15Y/95V/149R/154C/156T/177P/186R/195G/224Y/226I/230D/266L/334R, 2V/15Y/95V/116N/144W/149R/154C/156L/159F/177P/186R/195G/224Y/226I, 15Y/95V/149R/154C/156A/177P/183V/185G/186R/195G/224Y/226I/230D, 2E/14K/15Y/95V/116N/149R/154C/156T/177P/186R/195G/224Y/226I/316L/326R/330M, 15Y/39Q/63V/95V/149R/154C/156A/177P/186R/195G/224Y/226I/230D/316L/345V, 15Y/69D/95V/138R/144W/149R/154C/156T/159F/177P/185K/186R/195G/202Y/224Y/226I/316L, 15Y/39Q/95V/149R/154C/156Q/177P/186R/195G/224Y/226I/257L/266L/316L/334R, 15Y/95V/149R/154C/156Q/177P/186R/195G/224Y/226I/257L/275N/334R, 15Y/95V/149R/154C/156T/177P/183V/186R/195G/224Y/226I, 15Y/89W/95V/149R/154C/156A/177P/185G/186R/195G/224Y/226I/230D/316L/345V, 2E/14K/15Y/95V/119T/149R/154C/156T/177P/186R/195G/224Y/226I, 15Y/95V/144W/149R/154C/156L/177P/186R/195G/224Y/226I/316L/330M, 15Y/39Q/81Y/95V/149R/154C/156T/177P/185G/186R/195G/224Y/226I/316L/334R, 15Y/89H/95V/149R/154C/156T/177P/186R/195G/224Y/226I/230D/266L/345V, 15Y/39Q/95V/149R/154C/156Q/177P/186R/195G/224Y/226I/230D/275N/316L, 15Y/39Q/95V/149R/154C/156Q/177P/186R/195G/224Y/226I/230D/266L/334R/335H, 15Y/95V/149R/154C/156Q/177P/185G/186R/195G/224Y/226I/275N/316L/330R, 15Y/95V/119T/149R/154C/156N/177P/186R/195G/202Y/224Y/226I, 15Y/95V/149R/154C/156T/177P/186R/195G/224Y/226I/230D/257L/275N/316L/330R/345V, 15Y/95V/149R/154C/156T/177P/186R/195G/224Y/226I/230D, 15Y/81Y/95V/149R/154C/156Q/177P/186R/195G/224Y/226I/330R/335H/345V, 15Y/95V/149R/154C/156A/177P/183V/186R/195G/224Y/226I/230D/266L, 15Y/39S/95V/149R/154C/156T/177P/185K/186R/195G/224Y/226I, 15Y/39Q/81Y/95V/149R/154C/156T/177P/183V/186R/195G/224Y/226I/275N/316L/334R/345V, 15Y/95V/149R/154C/156A/177P/186R/195G/224Y/226I/230D/257L/316L, 15Y/39S/95V/116N/149R/154C/156N/177P/186R/195G/224Y/226I, or 15Y/95V/149R/154C/156T/177P/183V/185G/186R/195G/224Y/226I/257L/275N/316L/330R/334R, wherein the amino acid positions are relative to the reference sequence corresponding to residues 12 to 346 of SEQ ID NO: 14, or relative to the reference sequence corresponding to SEQ ID NO: 14.

In some embodiments, the amino acid sequence of the engineered RNA ligase comprises at least a substitution or substitution set provided in Table 10.1, 11.1, and 12.1, wherein the amino acid positions are relative to the reference sequence corresponding to residues 12 to 346 of SEQ ID NO: 14, or relative to the reference sequence corresponding to SEQ ID NO: 14.

In some embodiments, the amino acid sequence of the engineered RNA ligase comprises at least a substitution at an amino acid position provided in Tables 5.1, 6.1, 7.1, 8.1, 9.1, 10.1, 11.1, and 12.1, wherein the amino acid positions are relative to the reference sequence corresponding to residues 12 to 346 of SEQ ID NO: 14, or relative to the reference sequence corresponding to SEQ ID NO: 14.

In some embodiments, the amino acid sequence of the engineered RNA ligase comprises at least one substitution set forth in Tables 5.1, 6.1, 7.1, 8.1, 9.1, 10.1, 11.1, and 12.1, wherein the amino acid positions are relative to the reference sequence corresponding to residues 12 to 346 of SEQ ID NO: 14, or relative to the reference sequence corresponding to SEQ ID NO: 14.

In some embodiments, the amino acid sequence of the engineered RNA ligase comprises at least a substitution or substitution set at the amino acid position(s) set forth in Tables 5.1, 6.1, 7.1, 8.1, 9.1, 10.1, 11.1, and 12.1, wherein the amino acid positions are relative to the reference sequence corresponding to residues 12 to 346 of SEQ ID NO: 14, or relative to the reference sequence corresponding to SEQ ID NO: 14.

In some embodiments, the amino acid sequence of the engineered RNA ligase comprises at least a substitution or substitution set of an RNA ligase variant set forth in Tables 5.1, 6.1, 7.1, 8.1, 9.1, 10.1, 11.1, and 12.1, wherein the amino acid positions are relative to the reference sequence corresponding to residues 12 to 346 of SEQ ID NO: 14, or relative to the reference sequence corresponding to SEQ ID NO: 14.

In some embodiments, the engineered RNA ligase comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 42 or 204, or to the reference sequence corresponding to SEQ ID NO: 42 or 204.

In some embodiments, the engineered RNA ligase comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 24-582, or to the reference sequence corresponding to an even-numbered SEQ ID NO. of SEQ ID NOs: 24-582.

In some embodiments, the engineered RNA ligase comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 42 or 204, or to the reference sequence corresponding to SEQ ID NO: 42 or 204, wherein the amino acid sequence comprises one or more substitutions relative to the reference sequence corresponding to residues 12 to 346 of SEQ ID NO: 42 or 204, or relative to the reference sequence corresponding to SEQ ID NO: 42 or 204.

In some embodiments, the engineered RNA ligase comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or more sequence identity to the reference sequence corresponding to residues 12 to 346 of an even numbered SEQ ID NO. of SEQ ID NOs: 24-582, or to the reference sequence corresponding to an even numbered SEQ ID NO. of SEQ ID NOs: 24-582, wherein the amino acid sequence comprises one or more substitutions relative to the reference sequence corresponding to residues 12 to 346 of SEQ ID NO: 42 or 204, or relative to the reference sequence corresponding to SEQ ID NO: 42 or 204.

In some embodiments, the amino acid sequence of the engineered RNA ligase comprises at least a substitution at amino acid position 2, 14, 15, 18, 33, 39, 63, 69, 81, 83, 85, 86, 89, 95, 116, 117, 119, 138, 142, 144, 149, 154, 156, 159, 171, 175, 177, 181, 185, 186, 195, 202, 212, 214, 224, 226, 230, 247, 256, 257, 260, 266, 275, 280, 283, 285, 288, 291, 296, 303, 306, 307, 310, 314, 315, 316, 317, 326, 330, 331, 333, 334, 335, 337, 339, 342, or 345, or combinations thereof, wherein the amino acid positions are relative to the reference sequence corresponding to residues 12 to 346 of SEQ ID NO: 42 or 204, or relative to the reference sequence corresponding to SEQ ID NO: 42 or 204.

In some embodiments, the amino acid sequence of the engineered RNA ligase comprises at least a substitution or an amino acid residue 2G/V/E, 14K, 15E/L/M/P/Y, 18I, 33L, 39Q/S, 63V, 69D, 81Y, 85L, 86G, 89H/I/M/W, 95V, 116N, 117D, 119T, 138R, 142R, 144W, 149R/T, 154C, 156A/L/N/Q/T, 159F, 171E, 175K, 177P, 181D, 183V, 185G/K, 186D/R, 195G, 202Y, 212I, 214I, 224Y, 226I, 230D, 247K, 256A, 257L, 260S, 266L, 275N, 280G/N, 283I, 285K, 288E, 291P, 296K, 303Q, 306L, 307E/Q, 310K, 314W, 315S/T, 316L, 317A, 326R, 330M/R, 331R/W, 333V, 334R, 335D/H, 337L, 339P, 342I, or 345V, or combinations thereof, wherein the amino acid positions are relative to the reference sequence corresponding to residues 12 to 346 of SEQ ID NO: 42 or 204, or relative to the reference sequence corresponding to SEQ ID NO: 42 or 204.

In some embodiments, the amino acid sequence of the engineered RNA ligase comprises at least a substitution at amino acid position 95 or 177, or combination thereof, wherein the amino acid positions are relative to the reference sequence corresponding to residues 12 to 346 of SEQ ID NO: 42 or 204, or relative to the reference sequence corresponding to SEQ ID NO: 42 or 204.

In some embodiments, the amino acid sequence of the engineered RNA ligase comprises at least an amino acid residue 95V or 177P, or combination thereof, wherein the amino acid positions are relative to the reference sequence corresponding to residues 12 to 346 of SEQ ID NO: 42 or 204, or relative to the reference sequence corresponding to SEQ ID NO: 42 or 204.

In some embodiments, the amino acid sequence of the engineered RNA ligase comprises at least a substitution at amino acid position 15, 95, 149, 154, 156, 177, 186, 195, 224, or 226, or combinations thereof, wherein the amino acid positions are relative to the reference sequence corresponding to residues 12 to 346 of SEQ ID NO: 42 or 204, or relative to the reference sequence corresponding to SEQ ID NO: 42 or 204.

In some embodiments, the amino acid sequence of the engineered RNA ligase comprises at least a substitution or an amino acid residue 15Y, 95V, 149R, 154C, 156T, 177P, 186R, 195G, 224Y, or 226I, or combinations thereof, wherein the amino acid positions are relative to the reference sequence corresponding to residues 12 to 346 of SEQ ID NO: 42 or 204, or relative to the reference sequence corresponding to SEQ ID NO: 42 or 204.

In some embodiments, the engineered RNA ligase comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 42, or to the reference sequence corresponding to SEQ ID NO: 42, wherein the amino acid sequence comprises one or more substitutions relative to the reference sequence corresponding to residues 12 to 346 of SEQ ID NO: 42, or relative to the reference sequence corresponding to SEQ ID NO: 42.

In some embodiments, the engineered RNA ligase comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 130-478, or to the reference sequence corresponding to an even-numbered SEQ ID NO. of SEQ ID NOs: 130-478, wherein the amino acid sequence comprises one or more substitutions relative to the reference sequence corresponding to residues 12 to 346 of SEQ ID NO: 42, or relative to the reference sequence corresponding to SEQ ID NO: 42.

In some embodiments, the amino acid sequence of the engineered RNA ligases comprises a substitution or substitution set at amino acid positions(s) 149/156/195, 15/149/186/224/317, 15/156/195/224/226/317/331, 15/195/317/331, 149/224/331, 15/195/224/226/317/331, 15/33/86/149/154/195/317, 15/86/154/156/186/195/224/226/331, 15/149/224/317/331, 15/154/156/186/317/331, 15/33/149/224/226/331, 15/186/224/226/317/331, 15/33/149/186/224/331, 195/224/331, 15/86/156/186/195/226/317/331, 15/195/22/331, 156/186/224/226/331, 15/149/154/156/224/226, 33/156/186/195/331, 15/86/149/154/156/317/331, 15/186/317/331, 15/86/149/154/195/331, 15/33/149/154/156/226, 15/149/154/186/317/331, 195/331, 15/86/156/186/195/331, 149/156/224/226/331, 15/149/154/156/186/317/331, 15/154/186/195, 15/86/195/224/226, 230, 15/33/156/331, 15/33/156/195/224/226, 33/149/156/317, 15/33/186/195, 334, 15/154/156/224/317, 15/149/186/331, 15/86/149/186/195/317, 15/33/154/195, 15/156/186/331, 15/149/186/317, 15/33/156/186/214/224/331, 15/86/186/224/226/317, 15/154/331, 15/149/154/226, 15/33/195/226, 15/224/317/331, 15/86/186/195/317, 226/317/331, 330, 89, 15/149/186, 156/195/331, 156, 15/86/156/186/331, 275, 15/33/186/224, 15/186/317, 15/33/186/226/317, 15/149/154/156/331, 15/154/226/317, 15/86/156/186/195/317, 257, 15/154/156/186, 33/186/224, 335, 15/86/156/186/195/224/331, 154/156/317, 39, 2, 15/224/226/331, 15/33/149/224, 89/307, 15/154/156/317, 15/186/331, 15/33/154/317, 15/33/195/224/317, 15/156/224/226, 154/186/331, 185, 15/18/149/186/195, 86/149/154/156, 15/33/149/331, 326, 202, 119, 212, 15/33/156/317, 280, 142, 15/149, 15/33/86/156/195, 315, 256, 15/33/156/186, 138, 117, 116, 316, 69, 310, 317, 15/195/331, 15/33/331, 314, or 144, wherein the amino acid positions are relative to the reference sequence corresponding to residues 12 to 346 of SEQ ID NO: 42, or relative to the reference sequence corresponding to SEQ ID NO: 42.

In some embodiments, the amino acid sequence of the engineered RNA ligases comprises a substitution or substitution set 149R/156T/195G, 15M/149T/186R/224Y/317A, 15P/156T/195G/224Y/226I/317A/331L, 15M/195G/317A/331R, 149R/224Y/331R, 15E/195G/224Y/226I/317A/331W, 15P/33L/86G/149R/154C/195G/317A, 15M/86G/154C/156T/186R/195G/224Y/226I/331L, 15P/149R/224Y/317A/331R, 15M/154C/156T/186R/317A/331R, 15M/33L/149R/224Y/226I/331W, 15E/186R/224Y/226I/317A/331R, 15Y/33L/149R/186R/224Y/331R, 195G/224Y/331R, 15P/86G/156T/186R/195G/226I/317A/331L, 15M/195G/226/331W, 156T/186R/224Y/226I/331R, 15Y/149R/154C/156T/224Y/226I, 33L/156T/186R/195G/331L, 15P/86G/149R/154C/156T/317A/331R, 15M/186D/317A/331R, 15P/86G/149R/154C/195G/331L, 15Y/33L/149T/154C/156T/226I, 15P/149R/154C/186R/317A/331L, 195G/331R, 15M/86G/156T/186R/195G/331W, 149R/156T/224Y/226I/331W, 15P/149R/154C/156T/186R/317A/331L, 15Y/154C/186D/195G, 15E/86G/195G/224Y/226I, 230D, 15P/33L/156T/331R, 15L/33L/156T/195G/224Y/226I, 33L/149R/156T/317A, 15E/33L/186R/195G, 334R, 15E/154C/156T/224Y/317A, 15Y/149R/186R/331W, 15Y/86G/149T/186R/195G/317A, 15P/33L/154C/195G, 15P/154C/156T/186R/317A/331R, 15M/156T/186R/331R, 15Y/149T/186D/317A, 15E/33L/156T/186R/214I/224Y/331W, 15E/86G/186R/224Y/226I/317A, 15P/154C/331R, 15Y/149T/154C/226I, 15Y/33L/195G/226I, 15M/224Y/317A/331W, 15E/86G/186R/195G/317A, 226I/317A/331L, 330R, 89W, 15P/149R/186D/331L, 15P/149R/186R, 89M, 156T/195G/331L, 156A, 15E/86G/156T/186R/331R, 195G/331W, 275N, 15P/33L/186R/224Y, 15E/186R/317A, 15M/33L/186R/226I/317A, 89G, 15Y/149R/154C/156T/331W, 15Y/154C/226I/317A, 15Y/86G/156T/186R/195G/317A, 257L, 15Y/154C/156T/186D, 33L/186R/224Y, 335H, 15Y/86G/156T/186R/195G/224Y/331W, 154C/156T/317A, 39Q, 2G, 15M/224Y/226I/331R, 15M/33L/149R/224Y, 89I/307Q, 15M/154C/156T/317A, 15E/186R/331W, 15P/33L/154C/317A, 89H, 15Y/33L/195G/224Y/317A, 15E/156T/224Y/226I, 154C/186R/331L, 185G, 15M/18I/149R/186R/195G, 86G/149R/154C/156T, 15P/33L/149R/331L, 326R, 202Y, 119T, 212I, 2V, 15E/149R/154C/156T/331L, 15P/33L/156T/317A, 15Y/154C/156T/317A, 156L, 335D, 156T, 2E, 280G, 142R, 15E/149R, 156Q, 15E/33L/86G/156T/195G, 315T, 185K, 39S, 256A, 15Y/33L/156T/186D, 138R, 117D, 116N, 316L, 15M/149R, 69D, 156N, 310K, 317A, 15Y/195G/331L, 15M/33L/331L, 330M, 314W, 144W, 15Y/149R, or 315S, wherein the amino acid positions are relative to the reference sequence corresponding to residues 12 to 346 of SEQ ID NO: 42, or relative to the reference sequence corresponding to SEQ ID NO: 42.

In some embodiments, the amino acid sequence of the engineered RNA ligase comprises at least a substitution or substitution set V149R/G156T/D195G, K15M/V149T/Y186R/N224Y/L317A, K15P/G156T/D195G/N224Y/K226I/L317A/I331L, K15M/D195G/L317A/I331R, V149R/N224Y/I331R, K15E/D195G/N224Y/K226I/L317A/I331W, K15P/G33L/K86G/V149R/A154C/D195G/L317A, K15M/K86G/A154C/G156T/Y186R/D195G/N224Y/K226I/I331L, K15P/V149R/N224Y/L317A/I331R, K15M/A154C/G156T/Y186R/L317A/I331R, K15M/G33L/V149R/N224Y/K226I/I331W, K15E/Y186R/N224Y/K226I/L317A/I331R, K15Y/G33L/V149R/Y186R/N224Y/I331R, D195G/N224Y/I331R, K15P/K86G/G156T/Y186R/D195G/K226I/L317A/I331L, K15M/D195G/K226I/I331W, G156T/Y186R/N224Y/K226I/I331R, K15Y/V149R/A154C/G156T/N224Y/K226I, G33L/G156T/Y186R/D195G/I331L, K15P/K86G/V149R/A154C/G156T/L317A/I331R, K15M/Y186D/L317A/I331R, K15P/K86G/V149R/A154C/D195G/I331L, K15Y/G33L/V149T/A154C/G156T/K226I, K15P/V149R/A154C/Y186R/L317A/I331L, D195G/I331R, K15M/K86G/G156T/Y186R/D195G/I331W, V149R/G156T/N224Y/K226I/I331W, K15P/V149R/A154C/G156T/Y186R/L317A/I331L, K15Y/A154C/Y186D/D195G, K15E/K86G/D195G/N224Y/K226I, N230D, K15P/G33L/G156T/I331R, K15L/G33L/G156T/D195G/N224Y/K226I, G33L/V149R/G156T/L317A, K15E/G33L/Y186R/D195G, Q334R, K15E/A154C/G156T/N224Y/L317A, K15Y/V149R/Y186R/I331W, K15Y/K86G/V149T/Y186R/D195G/L317A, K15P/G33L/A154C/D195G, K15P/A154C/G156T/Y186R/L317A/I331R, K15M/G156T/Y186R/I331R, K15Y/V149T/Y186D/L317A, K15E/G33L/G156T/Y186R/V214I/N224Y/I331W, K15E/K86G/Y186R/N224Y/K226I/L317A, K15P/A154C/I331R, K15Y/V149T/A154C/K226I, K15Y/G33L/D195G/K226I, K15M/N224Y/L317A/I331W, K15E/K86G/Y186R/D195G/L317A, K226I/L317A/I331L, T330R, K89W, K15P/V149R/Y186D/I331L, K15P/V149R/Y186R, K89M, G156T/D195G/I331L, G156A, K15E/K86G/G156T/Y186R/I331R, D195G/I331W, T275N, K15P/G33L/Y186R/N224Y, K15E/Y186R/L317A, K15M/G33L/Y186R/K226I/L317A, K89G, K15Y/V149R/A154C/G156T/I331W, K15Y/A154C/K226I/L317A, K15Y/K86G/G156T/Y186R/D195G/L317A, V257L, K15Y/A154C/G156T/Y186D, G33L/Y186R/N224Y, N335H, K15Y/K86G/G156T/Y186R/D195G/N224Y/I331W, A154C/G156T/L317A, H39Q, H2G, K15M/N224Y/K226I/I331R, K15M/G33L/V149R/N224Y, K89I/N307Q, K15M/A154C/G156T/L317A, K15E/Y186R/I331W, K15P/G33L/A154C/L317A, K89H, K15Y/G33L/D195G/N224Y/L317A, K15E/G156T/N224Y/K226I, A154C/Y186R/I331L, N185G, K15M/S18I/V149R/Y186R/D195G, K86G/V149R/A154C/G156T, K15P/G33L/V149R/I331L, K326R, H202Y, Q119T, T212I, H2V, K15E/V149R/A154C/G156T/I331L, K15P/G33L/G156T/L317A, K15Y/A154C/G156T/L317A, G156L, N335D, G156T, H2E, S280G, K142R, K15E/V149R, G156Q, K15E/G33L/K86G/G156T/D195G, D315T, N185K, H39S, K256A, K15Y/G33L/G156T/Y186D, S138R, G117D, G116N, M316L, K15M/V149R, T690, G156N, A310K, L317A, K15Y/D195G/I331L, K15M/G33L/I331L, T330M, H314W, Y144W, K15Y/V149R, or D315S, wherein the amino acid positions are relative to the reference sequence corresponding to residues 12 to 346 of SEQ ID NO: 42, or relative to the reference sequence corresponding to SEQ ID NO: 42.

In some embodiments, the amino acid sequence of the engineered RNA ligases comprises at least a substitution set at amino acid positions 15/149/154/156/186/195/331, 149/154/186/195/224/317/331, 15/33/149/154/186/195, 149/156/186/195/224/226/317, 15/149/156/195/224/226, 15/33/86/149/154/156/186/195/226/331, 15/149/156/186/195/224/317/331, 15/33/149/154/195/317, 15/149/154/195/224/317, 15/149/156/186/224/226/317/331, 15/33/149/154/156/186/195/224/226/317/331, 149/195/224, 86/149/154/156/186/195/331, 15/33/149/156/195/224/317/331, 15/149/154/156/186/195/317/331, 15/33/149/154/156/186/224/331, 15/149/154/156/195/224, 15/149/154/156/195/224/226, 15/149/154/156/186/195/224/317, 33/149/156/186/224/331, 15/33/86/149/195/224/331, 15/86/149/154/156/186/195/331, 149/186/195/331, 15/154/156/186/226/331, 15/149/154/186/195/224/226, 15/149/154/156/195/224/331, 15/33/149/154/156/195/317/331, 15/33/154/156/186/195/224/317/331, 15/149/186/195/224/331, 15/33/149/154/156/195/224/331, 15/149/154/156/224/226/317/331, 15/33/149/156/195/331, 15/149/154/156/186/224/226/331, 154/156/195/224/226, 15/33/86/149/154/156/186/195/224/317/331, 15/149/154/156/186/195/224/226, 15/33/149/195/317/331, 15/149/154/224/331, 149/154/156/195/224/226/331, 15/149/154/195/331, 15/149/154/156/186/195, 15/33/149/154/156/186/195, 15/33/86/149/154/195/224/317/331, or 149/186/195/224/226/331, wherein the amino acid positions are relative to the reference sequence corresponding to residues 12 to 346 of SEQ ID NO: 42, or relative to the reference sequence corresponding to SEQ ID NO: 42.

In some embodiments, the amino acid sequence of the engineered RNA ligases comprises at least a substitution set 15M/149R/154C/156T/186R/195G/331R, 149R/154C/186D/195G/224Y/317A/331R, 15M/33L/149T/154C/186R/195G, 149R/156T/186R/195G/224Y/226I/317A, 15Y/149R/156T/195G/224Y/226I, 15Y/33L/86G/149T/154C/156T/186R/195G/226I/331W, 15Y/149R/156T/186R/195G/224Y/317A/331W, 15M/33L/149T/154C/195G/317A, 15M/149R/154C/195G/224Y/317A, 15Y/149R/156T/186D/224Y/226I/317A/331R, 15M/149T/154C/156T/186D/195G/331L, 15Y/33L/149R/154C/156T/186R/195G/224Y/226I/317A/331R, 149R/195G/224Y, 86G/149T/154C/156T/186R/195G/331L, 15P/33L/149R/156T/195G/224Y/317A/331L, 15P/149T/154C/156T/186D/195G/317A/331L, 15Y/33L/149T/154C/156T/186D/224Y/331L, 15P/149R/156T/186R/195G/224Y/317A/331W, 15Y/149R/154C/156T/195G/224Y, 15M/149T/154C/156T/195G/224Y/226I, 15E/149R/154C/156T/186R/195G/224Y/317A, 33L/149T/156T/186R/224Y/331L, 15Y/33L/86G/149R/195G/224Y/331W, 15E/86G/149R/154C/156T/186R/195G/331W, 149R/186R/195G/331W, 15E/154C/156T/186D/226I/331R, 15Y/149T/154C/186R/195G/224Y/226I, 15P/149R/154C/156T/195G/224Y/331W, 15E/33L/149R/154C/156T/195G/317A/331L, 15E/33L/154C/156T/186R/195G/224Y/317A/331L, 15P/149R/186D/195G/224Y/331L, 15P/33L/149R/154C/156T/195G/224Y/331W, 15P/149T/154C/156T/224Y/226I/317A/331W, 15P/33L/149T/156T/195G/331W, 15P/149T/154C/156T/186D/224Y/226I/331L, 154C/156T/195G/224Y/226I, 15Y/33L/86G/149R/154C/156T/186D/195G/224Y/317A/331W, 15Y/149R/154C/156T/186R/195G/224Y/226I, 15P/33L/149R/195G/317A/331L, 15E/149T/154C/224Y/331R, 149T/154C/156T/195G/224Y/226I/331R, 15M/149R/154C/195G/331L, 15Y/149R/154C/156T/186R/195G, 15E/149T/154C/156T/186R/195G/331R, 15M/33L/149T/154C/156T/186R/195G, 15P/33L/86G/149R/154C/195G/224Y/317A/331R, or 149R/186D/195G/224Y/226I/331R, wherein the amino acid positions are relative to the reference sequence corresponding to residues 12 to 346 of SEQ ID NO: 42, or relative to the reference sequence corresponding to SEQ ID NO: 42.

In some embodiments, the amino acid sequence of the engineered RNA ligase comprises at least a substitution set K15M/V149R/A154C/G156T/Y186R/D195G/I331R, V149R/A154C/Y186D/D195G/N224Y/L317A/I331R, K15M/G33L/V149T/A154C/Y186R/D195G, V149R/G156T/Y186R/D195G/N224Y/K226I/L317A, K15Y/V149R/G156T/D195G/N224Y/K226I, K15Y/G33L/K86G/V149T/A154C/G156T/Y186R/D195G/K226I/I331W, K15Y/V149R/G156T/Y186R/D195G/N224Y/L317A/I331W, K15M/G33L/V149T/A154C/D195G/L317A, K15M/V149R/A154C/D195G/N224Y/L317A, K15Y/V149R/G156T/Y186D/N224Y/K226I/L317A/I331R, K15M/V149T/A154C/G156T/Y186D/D195G/I331L, K15Y/G33L/V149R/A154C/G156T/Y186R/D195G/N224Y/K226I/L317A/I331R, V149R/D195G/N224Y, K86G/V149T/A154C/G156T/Y186R/D195G/I331L, K15P/G33L/V149R/G156T/D195G/N224Y/L317A/I331L, K15P/V149T/A154C/G156T/Y186D/D195G/L317A/I331L, K15Y/G33L/V149T/A154C/G156T/Y186D/N224Y/I331L, K15P/V149R/G156T/Y186R/D195G/N224Y/L317A/I331W, K15Y/V149R/A154C/G156T/D195G/N224Y, K15M/V149T/A154C/G156T/D195G/N224Y/K226I, K15E/V149R/A154C/G156T/Y186R/D195G/N224Y/L317A, G33L/V149T/G156T/Y186R/N224Y/I331L, K15Y/G33L/K86G/V149R/D195G/N224Y/I331W, K15E/K86G/V149R/A154C/G156T/Y186R/D195G/I331W, V149R/Y186R/D195G/I331W, K15E/A154C/G156T/Y186D/K226I/I331R, K15Y/V149T/A154C/Y186R/D195G/N224Y/K226I, K15P/V149R/A154C/G156T/D195G/N224Y/I331W, K15E/G33L/V149R/A154C/G156T/D195G/L317A/I331L, K15E/G33L/A154C/G156T/Y186R/D195G/N224Y/L317A/I331L, K15P/V149R/Y186D/D195G/N224Y/I331L, K15P/G33L/V149R/A154C/G156T/D195G/N224Y/I331W, K15P/V149T/A154C/G156T/N224Y/K226I/L317A/I331W, K15P/G33L/V149T/G156T/D195G/I331W, K15P/V149T/A154C/G156T/Y186D/N224Y/K226I/I331L, A154C/G156T/D195G/N224Y/K226I, K15Y/G33L/K86G/V149R/A154C/G156T/Y186D/D195G/N224Y/L317A/I331W, K15Y/V149R/A154C/G156T/Y186R/D195G/N224Y/K226I, K15P/G33L/V149R/D195G/L317A/I331L, K15E/V149T/A154C/N224Y/I331R, V149T/A154C/G156T/D195G/N224Y/K226I/I331R, K15M/V149R/A154C/D195G/I331L, K15Y/V149R/A154C/G156T/Y186R/D195G, K15E/V149T/A154C/G156T/Y186R/D195G/I331R, K15M/G33L/V149T/A154C/G156T/Y186R/D195G, K15P/G33L/K86G/V149R/A154C/D195G/N224Y/L317A/I331R, or V149R/Y186D/D195G/N224Y/K226I/I331R, wherein the amino acid positions are relative to the reference sequence corresponding to residues 12 to 346 of SEQ ID NO: 42, or relative to the reference sequence corresponding to SEQ ID NO: 42.

In some embodiments, the engineered RNA ligase comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 204, or to the reference sequence corresponding to SEQ ID NO: 204, wherein the amino acid sequence comprises one or more substitutions relative to the reference sequence corresponding to residues 12 to 346 of SEQ ID NO: 204, or relative to the reference sequence corresponding to SEQ ID NO: 204.

In some embodiments, the engineered RNA ligase comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 480-582, to the reference sequence corresponding to an even-numbered SEQ ID NO. of SEQ ID NOs: 480-582, wherein the amino acid sequence comprises one or more substitutions relative to the reference sequence corresponding to residues 12 to 346 of SEQ ID NO: 204, or relative to the reference sequence corresponding to SEQ ID NO: 204.

In some embodiments, the amino acid sequence of the engineered RNA ligase comprises at least a substitution or substitution set at amino acid position(s) 2/39/69/144/156/316/330, 2/39/69/119/185/316, 69/212/256/330, 39/119/256/316/326/330, 69/116/119/185/330, 2/39/119/144/156/159/202/256/316/326/330, 81/183/185/230, 2/39/69/116/144/156/185, 2/14/39/69/116/119/159/185/256/326, 2/116/119/316, 116/119/144/185/314/316, 39/69/116/144/181/185/256/330, 2/39/69/119/144/156/185/314, 14/39/69/144/156/212/256/314/316/326/330, 39/69/144/156/185/316/330, 156/185/334/335, 39/63/156/230/257/275/330/335, 156/266/316/330/334/335, 39/63/156/183/185/230/330/334, 69/144/316, 116/119/156/202/212/326/330, 39/69/119/138/181/185/316/326, 39/81/156/230, 2/14/69/116/119/144/159/326/330, 39/156/334/345, 230/266/334, 2/116/144/156/159, 156/183/185/230, 2/14/116/316/326/330, 39/63/156/230/316/345, 69/138/144/159/185/202/316, 39/156/257/266/316/334, 156/257/275/334, 183, 89/156/185/230/316/345, 2/14/119, 144/156/316/330, 39/81/185/316/334, 89/230/266/345, 39/156/230/275/316, 39/156/230/266/334/335, 156/185/275/316/330, 119/156/202, 230/257/275/316/330/345, 230, 81/156/330/335/345, 156/183/230/266, 39/185, 39/81/183/275/316/334/345, 156/230/257/316, 39/116/156, or 183/185/257/275/316/330/334, wherein the amino acid positions are relative to the reference sequence corresponding to residues 12 to 346 of SEQ ID NO: 204, or relative to the reference sequence corresponding to SEQ ID NO: 204.

In some embodiments, the amino acid sequence of the engineered RNA ligase comprises at least a substitution or substitution set 2E/39S/69D/144W/156L/316L/330M, 2V/39S/69D/119T/185K/316L, 69D/212I/256A/330M, 39S/119T/256A/316L/326R/330M, 69D/116N/119T/185K/330M, 2E/39S/119T/144W/156N/159F/202Y/256A/316L/326R/330M, 81Y/183V/185G/230D, 2V/39S/69D/116N/144W/156L/185K, 2E/14K/39S/69D/116N/119T/159F/185K/256A/326R, 2E/116N/119T/316L, 116N/119T/144W/185K/314W/316L, 39S/69D/116N/144W/181D/185K/256A/330M, 2E/39S/69D/119T/144W/156L/185K/314W, 14K/39S/69D/144W/156N/212I/256A/314W/316L/326R/330M, 39S/69D/144W/156L/185K/316L/330M, 156Q/185G/334R/335H, 39Q/63V/156Q/230D/257L/275N/330R/335H, 156Q/266L/316L/330R/334R/335H, 39Q/63V/156Q/183V/185G/230D/330R/334R, 69D/144W/316L, 116N/119T/156L/202Y/212I/326R/330M, 39S/69D/119T/138R/181D/185K/316L/326R, 39Q/81Y/156Q/230D, 2V/14K/69D/116N/119T/144W/159F/326R/330M, 39Q/156A/334R/345V, 230D/266L/334R, 2V/116N/144W/156L/159F, 156A/183V/185G/230D, 2E/14K/116N/316L/326R/330M, 39Q/63V/156A/230D/316L/345V, 69D/138R/144W/159F/185K/202Y/316L, 39Q/156Q/257L/266L/316L/334R, 156Q/257L/275N/334R, 183V, 89W/156A/185G/230D/316L/345V, 2E/14K/119T, 144W/156L/316L/330M, 39Q/81Y/185G/316L/334R, 89H/230D/266L/345V, 39Q/156Q/230D/275N/316L, 39Q/156Q/230D/266L/334R/335H, 156Q/185G/275N/316L/330R, 119T/156N/202Y, 230D/257L/275N/316L/330R/345V, 230D, 81Y/156Q/330R/335H/345V, 156A/183V/230D/266L, 39S/185K, 39Q/81Y/183V/275N/316L/334R/345V, 156A/230D/257L/316L, 39S/116N/156N, or 183V/185G/257L/275N/316L/330R/334R, wherein the amino acid positions are relative to the reference sequence corresponding to residues 12 to 346 of SEQ ID NO: 204, or relative to the reference sequence corresponding to SEQ ID NO: 204.

In some embodiments, the amino acid sequence of the engineered RNA ligase comprises at least a substitution or substitution set H2E/H39S/T69D/Y144W/T156L/M316L/T330M, H2V/H39S/T69D/Q119T/N185K/M316L, T69D/T212I/K256A/T330M, H39S/Q119T/K256A/M316L/K326R/T330M, T69D/G116N/Q119T/N185K/T330M, H2E/H39S/Q119T/Y144W/T156N/L159F/H202Y/K256A/M316L/K326R/T330M, H81Y/I183V/N185G/N230D, H2V/H39S/T69D/G116N/Y144W/T156L/N185K, H2E/A14K/H39S/T69D/G116N/Q119T/L159F/N185K/K256A/K326R, H2E/G116N/Q119T/M316L, G116N/QI 19T/Y144W/N185K/H314W/M316L, H39S/T69D/G116N/Y144W/K181D/N185K/K256A/T330M, H2E/H39S/T69D/Q119T/Y144W/T156L/N185K/H314W, A14K/H39S/T69D/Y144W/T156N/T212/K256A/H314W/M316L/K326R/T330M, H39S/T69D/Y144W/T156L/N185K/M316L/T330M, T156Q/N185G/Q334R/N335H, H39Q/I63V/T156Q/N230D/V257L/T275N/T330R/N335H, T156Q/V266L/M316L/T330R/Q334R/N335H, H39Q/I63V/T156Q/I183V/N185G/N230D/T330R/Q334R, T69D/Y144W/M316L, G116N/Q119T/T156L/H202Y/T212I/K326R/T330M, H39S/T69D/Q119T/S138R/K181D/N185K/M316L/K326R, H39Q/H81Y/T156Q/N230D, H2V/A14K/T69D/G116N/Q119T/Y44W/L159F/K326R/T330M, H39Q/T156A/Q334R/L345V, N230D/V266L/Q334R, H2V/G116N/Y144W/T156L/L159F, T156A/I183V/N185G/N230D, H2E/A14K/G116N/M316L/K326R/T330M, H39Q/I63V/T156A/N230D/M316L/L345V, T69D/S138R/Y144W/L159F/N185K/H202Y/M316L, H39Q/T156Q/V257L/V266L/M316L/Q334R, T156Q/V257L/T275N/Q334R, I183V, K89W/T156A/N185G/N230D/M316L/L345V, H2E/A14K/Q I19T, Y144W/T156L/M316L/T330M, H39Q/H81Y/N185G/M316L/Q334R, K89H/N230D/V266L/L345V, H39Q/T156Q/N230D/T275N/M316L, H39Q/T156Q/N230D/V266L/Q334R/N335H, T156Q/N185G/T275N/M316L/T330R, Q119T/T156N/H202Y, N230D/V257L/T275N/M316L/T330R/L345V, N230D, H81Y/T156Q/T330R/N335H/L345V, T156A/I183V/N230D/V266L, H39S/N185K, H39Q/H81Y/I183V/T275N/M316L/Q334R/L345V, T156A/N230D/V257L/M316L, H39S/G116N/T156N, or I183V/N185G/V257L/T275N/M316L/T330R/Q334R, wherein the amino acid positions are relative to the reference sequence corresponding to residues 12 to 346 of SEQ ID NO: 204, or relative to the reference sequence corresponding to SEQ ID NO: 204.

In some embodiments, the amino acid sequence of the engineered RNA ligase comprises at least a substitution at an amino acid position provided in Tables 6.1, 7.1, 8.1, 9.1, 10.1, 11.1, and 12.1, wherein the amino acid positions are relative to the reference sequence corresponding to residues 12 to 346 of SEQ ID NO: 42 or 204, or relative to the reference sequence corresponding to SEQ ID NO: 42 or 204, as provided in each of the Tables.

In some embodiments, the amino acid sequence of the engineered RNA ligase comprises at least one substitution provided in Tables 6.1, 7.1, 8.1, 9.1, 10.1, 11.1, and 12.1, wherein the amino acid positions are relative to the reference sequence corresponding to residues 12 to 346 of SEQ ID NO: 42 or 204, or relative to the reference sequence corresponding to SEQ ID NO: 42 or 204, as provided in each of the Tables.

In some embodiments, the amino acid sequence of the engineered RNA ligase comprises at least a substitution or substitution set at the amino acid position(s) provided in Tables 6.1, 7.1, 8.1, 9.1, 10.1, 11.1, and 12.1, wherein the amino acid positions are relative to the reference sequence corresponding to residues 12 to 346 of SEQ ID NO: 42 or 204, or relative to the reference sequence corresponding to SEQ ID NO: 42 or 204, as provided in each of the Tables.

In some embodiments, the amino acid sequence of the engineered RNA ligase comprises at least a substitution or substitution set of an RNA ligase variant provided in Tables 6.1, 7.1, 8.1, 9.1, 10.1, 11.1, and 12.1, wherein the amino acid positions are relative to the reference sequence corresponding to residues 12 to 346 of SEQ ID NO: 42 or 204, or relative to the reference sequence corresponding to SEQ ID NO: 42 or 204, as provided in each of the Tables.

In some embodiments, the engineered RNA ligase comprises an amino acid sequence having least 80%, 81%, 82%, 83%, 84%, 85%, 86%, 87%, 88%, 89%, 90%, 91%, 92%, 93%, 94%, 95%, 96%, 97%, 98%, 99% or more sequence identity to a reference sequence comprising a substitution or substitution set provided in Tables 5.1, 6.1, 7.1, 8.1, 9.1, 10.1, 11.1, and 12.1, wherein the amino acid positions are relative to the reference sequence corresponding to residues 12 to 346 of SEQ ID NO: 14, 42, or 204, or relative to the reference sequence corresponding to SEQ ID NO: 14, 42, or 204.

In some embodiments, the engineered RNA ligase comprises an amino acid sequence having least 80%, 81%, 82%, 83%, 84%, 85%, 86%, 87%, 88%, 89%, 90%, 91%, 92%, 93%, 94%, 95%, 96%, 97%, 98%, 99% or more sequence identity to the sequence comprising residues 12 to 346 of an engineered RNA ligase set forth in Tables 5.1, 6.1, 7.1, 8.1, 9.1, 10.1, 11.1, and 12.1, or to the sequence of an engineered RNA ligase set forth in Tables 5.1, 6.1, 7.1, 8.1, 9.1, 10.1, 11.1, and 12.1.

In some embodiments, the engineered RNA ligase comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or more sequence identity to the sequence corresponding to residues 12 to 346 of SEQ ID NO: 24, 26, 28, 30, 32, 34, 336, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 568, 570, 572, 574, 576, 578, 580, or 582.

In some embodiments, the engineered RNA ligase comprises an amino acid sequence having at least 80%, 85%, 86%, 87%, 88%, 89%, 90%, 91%, 92%, 93%, 94%, 95%, 96%, 97%, 98%, 99% or more sequence identity to the sequence corresponding to SEQ ID NO: 24, 26, 28, 30, 32, 34, 336, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, or 582.

In some embodiments, the engineered RNA ligase comprises an amino acid sequence comprising residues 12 to 346 of an even numbered SEQ ID NO. of SEQ ID NOs: 24-582, or an amino acid sequence comprising an even numbered SEQ ID NO. of SEQ ID NOs: 24-582. In some embodiments, the amino acid sequence of the engineered RNA ligase optionally includes 1, 2, 3, 4, 5, 6, 7, 8, 9, or up to 10 substitutions, insertions, and/or deletions. In some embodiments, the amino acid sequence of the engineered RNA ligase optionally includes 1, 2, 3, 4, 5, 6, 7, 8, 9, or up to 10 substitutions. In some embodiments, the amino acid sequence of the engineered RNA ligase optionally includes 1, 2, 3, 4, or 5 substitutions, insertions, and/or deletions. In some embodiments, the amino acid sequence of the engineered RNA ligase optionally includes 1, 2, 3, 4, or 5 substitutions.

In some embodiments, the engineered RNA ligase comprises an amino acid sequence comprising residues 12 to 346 of SEQ ID NO: 24, 26, 28, 30, 32, 34, 336, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, or 582. In some embodiments, the amino acid sequence of the engineered RNA ligase optionally includes 1, 2, 3, 4, 5, 6, 7, 8, 9, or up to 10 substitutions, insertions, and/or deletions. In some embodiments, the amino acid sequence of the engineered RNA ligase optionally includes 1, 2, 3, 4, 5, 6, 7, 8, 9, or up to 10 substitutions. In some embodiments, the amino acid sequence of the engineered RNA ligase optionally includes 1, 2, 3, 4, or 5 substitutions, insertions, and/or deletions.

In some embodiments, the amino acid sequence of the engineered RNA ligase optionally includes 1, 2, 3, 4, or 5 substitutions.

In some embodiments, the engineered RNA ligase comprises an amino acid sequence comprising SEQ ID NO: 24, 26, 28, 30, 32, 34, 336, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, or 582.

In some embodiments, the amino acid sequence of the engineered RNA ligase optionally includes 1, 2, 3, 4, 5, 6, 7, 8, 9, or up to 10 substitutions, insertions, and/or deletions. In some embodiments, the amino acid sequence of the engineered RNA ligase optionally includes 1, 2, 3, 4, 5, 6, 7, 8, 9, or up to 10 substitutions. In some embodiments, the amino acid sequence of the engineered RNA ligase optionally includes 1, 2, 3, 4, or 5 substitutions, insertions, and/or deletions. In some embodiments, the amino acid sequence of the engineered RNA ligase optionally includes 1, 2, 3, 4, or 5 substitutions.

In some embodiments, the engineered RNA ligase comprises an amino acid sequence comprising residues 12 to 346 of SEQ ID NO: 42 or 204, or an amino acid sequence comprising SEQ ID NO: 42 or 204. In some embodiments, the amino acid sequence of the engineered RNA ligase optionally includes 1, 2, 3, 4, 5, 6, 7, 8, 9, or up to 10 substitutions, insertions, and/or deletions. In some embodiments, the amino acid sequence of the engineered RNA ligase optionally includes 1, 2, 3, 4, 5, 6, 7, 8, 9, or up to 10 substitutions. In some embodiments, the amino acid sequence of the engineered RNA ligase optionally includes 1, 2, 3, 4, or 5 substitutions, insertions, and/or deletions. In some embodiments, the amino acid sequence of the engineered RNA ligase optionally includes 1, 2, 3, 4, or 5 substitutions.

In some of the foregoing embodiments, the engineered RNA ligase polypeptide has 1, 2, 3, 4, or up to 5 substitutions in the amino acid sequence. In some embodiments, the engineered RNA ligase polypeptide has 1, 2, 3, or 4 substitutions in the amino acid sequence. In some embodiments, the substitutions comprises non-conservative or conservative substitutions. In some embodiments, the substitutions comprises conservative substitutions. In some embodiments, the substitutions comprises non-conservative substitutions. In some embodiments, guidance on non-conservative and conservative substitutions are provided by the variants disclosed herein.

In some embodiments, the engineered RNA ligase of the present disclosure has RNA ligase 2 activity, particularly with the improved or enhanced properties described herein. In some embodiments, the engineered RNA ligase has at least one improved or enhanced property as compared to a reference RNA ligase. Exemplary improved properties are provided in the Examples.

In some embodiments, the engineered RNA ligase has increased activity as compared to the reference RNA ligase. In some embodiments, the engineered RNA ligase has at least 1.1 fold, 1.2 fold, 1.3 fold, 1.4 fold, 1.5 fold, 2-fold, 3-fold, 4-fold, 5-fold, 10-fold, or more fold activity compared to the reference RNA ligase. In some embodiments, the increased activity is on modified polynucleotide substrates, as discussed herein and in the Examples.

In some embodiments, the engineered RNA ligase exhibits increased product yield as compared to the reference RNA ligase. In some embodiments, the engineered RNA ligase exhibits increased product yield at substrate concentration of 10 uM to 400 uM, 20 uM to 350 uM, 50 uM to 300 uM, or 100 uM to 250 uM, preferably 200 to 400 uM as compared to the reference RNA ligase. In some embodiments, the engineered RNA ligase exhibits increased product yield at substrate concentration of 10 uM, 20 uM, 50 uM, 100 uM, 150 uM, 200 uM, 250 uM, 300 uM, 350 uM, or 400 uM or higher as compared to the reference RNA ligase.

In some embodiments, the engineered RNA ligase has increased stability as compared to the reference RNA ligase. In some embodiments, the engineered RNA ligase has increased thermostability as compared to the reference RNA ligase.

In some embodiments, the engineered RNA ligase has increased activity on polynucleotides substrates with phosphorothioate internucleotide linkages as compared to the reference RNA ligase. In some embodiments, the engineered RNA ligase has increased activity or product yield on polynucleotides substrates with 2′-modifications (e.g., 2′-O-methyl and/or 2′-fluoro) as compared to the reference RNA ligase.

In some embodiments, the reference RNA ligase has the sequence corresponding to residues 12 to 346 of SEQ ID NO: 14, 42, or 204, or the sequence corresponding to SEQ ID NO: 14, 42, or 204. In some embodiments, the reference RNA ligase has the sequence corresponding to residues 12 to 346 of SEQ ID NO: 14, or the sequence corresponding to SEQ ID NO: 14.

In some embodiments, the engineered RNA ligase has one or more improved property selected from i) increased activity, ii) increased stability, iii) increased thermostability, iv) increased product yield, v) increased activity on polynucleotides with phosphorothioate internucleotide linkages, vi) increased activity on oligonucleotides with 2′-modifications, vii) increased substrate tolerance, or any combination of i), ii), iii), iv), v), vi), and vii), compared to a reference RNA ligase. In some embodiments, the reference RNA ligase has the sequence corresponding to residues 12 to 346 of SEQ ID NO: 14, 42, or 204, or the sequence corresponding to SEQ ID NO: 14, 42, or 204. In some embodiments, the reference RNA ligase has the sequence corresponding to residues 12 to 346 of SEQ ID NO: 14, or the sequence corresponding to SEQ ID NO: 14.

In some embodiments, the present disclosure further provides an engineered RNA ligase comprising an amino acid sequence having at least 80%, 85%, 86%, 87%, 88%, 89%, 90%, 91%, 92%, 93%, 94%, 95%, 96%, 97%, 98%, 99% or more sequence identity to the sequence corresponding to:
residues 12 to 345 of SEQ ID NO: 2;
residues 12 to 343 of SEQ ID NO: 4;
residues 12 to 250 of SEQ ID NO: 6;
residues 12 to 346 of SEQ ID NO: 8;
residues 12 to 345 of SEQ ID NO: 10;
residues 12 to 345 of SEQ ID NO: 12;
residues 12 to 346 of SEQ ID NO: 14;
residues 12 to 346 of SEQ ID NO: 16;
residues 12 to 350 of SEQ ID NO: 18;
residues 12 to 350 of SEQ ID NO: 20; or
residues 12 to 344 of SEQ ID NO: 22.

In some embodiments, the engineered RNA ligase comprises an amino acid sequence having at least 80%, 85%, 86%, 87%, 88%, 89%, 90%, 91%, 92%, 93%, 94%, 95%, 96%, 97%, 98%, 99% or more sequence identity to the sequence corresponding to SEQ ID NO: 2, 4, 6, 8, 10, 12, 14, 16, 18, 20 or 22.

In some embodiments, the engineered RNA ligase comprises an amino acid sequence comprising:
residues 12 to 345 of SEQ ID NO: 2;
residues 12 to 343 of SEQ ID NO: 4;
residues 12 to 250 of SEQ ID NO: 6;
residues 12 to 346 of SEQ ID NO: 8;
residues 12 to 345 of SEQ ID NO: 10;
residues 12 to 345 of SEQ ID NO: 12;
residues 12 to 346 of SEQ ID NO: 14;
residues 12 to 346 of SEQ ID NO: 16;
residues 12 to 350 of SEQ ID NO: 18;
residues 12 to 350 of SEQ ID NO: 20; or residues 12 to 344 of SEQ ID NO: 22.

In some embodiments, the engineered RNA ligase comprises an amino acid sequence comprising SEQ ID NO: 2, 4, 6, 8, 10, 12, 14, 16, 18, 20 or 22.

In some embodiments, the engineered RNA ligase is expressed as a fusion protein. In some embodiments, the engineered RNA ligase described herein can be fused to a variety of polypeptide sequences, such as, by way of example and not limitation, polypeptide tags that can be used for detection and/or purification. In some embodiments, the fusion protein of the engineered RNA ligase comprises a glycine-histidine or histidine-tag (His-tag). In some embodiments, the fusion protein of the engineered RNA ligase comprises an epitope tag, such as c-myc, FLAG, V5, or hemagglutinin (HA). In some embodiments, the fusion protein of the engineered RNA ligase comprises a GST, SUMO, Strep, MBP, or GFP tag. In some embodiments, the fusion is to the amino (N-) terminus of engineered RNA ligase polypeptide. In some embodiments, the fusion is to the carboxy (C-) terminus of the engineered RNA ligase polypeptide.

In some embodiments, the engineered RNA ligase polypeptide described herein is an isolated composition. In some embodiments, the engineered RNA ligase polypeptide is purified, as further discussed herein. In some embodiments, the engineered RNA ligase is provided in solution, as a lyophilizate, or immobilized on a substrate, as further discussed herein.

In some embodiments, the present disclosure further provides functional fragments or biologically active fragments of engineered RNA ligase polypeptides described herein. Thus, for each and every embodiment herein of an engineered RNA ligase, a functional fragment or biologically active fragment of the engineered RNA ligase is provided herewith. In some embodiments, a functional fragment or biologically active fragments of an engineered RNA ligase comprises at least about 90%, at least about 95%, at least about 96%, at least about 97%, at least about 98%, or at least about 99% of the activity of the RNA ligase polypeptide from which it was derived (i.e., the parent RNA ligase). In some embodiments, functional fragments or biologically active fragments comprise at least 90%, 91%, 92%, 93%, 94%, 95%, 96%, 97%, 98%, or 99% of the parent sequence of the RNA ligase. In some embodiments, the functional fragment will be truncated by less than 5, less than 10, less than 15, less than 10, less than 25, less than 30, less than 35, less than 40, less than 45, less than 50 amino acids, less than 55 amino acids, less than 60 amino acids, less than 65 amino acids, or less than 70 amino acids.

In some embodiments, a functional fragment of an engineered RNA ligase herein comprises at least 70%, 75%, 80%, 81%, 82%, 83%, 84%, 85%, 90%, 91%, 92%, 93%, 94%, 95%, 96%, 97%, 98%, or 99% of the parent sequence of the engineered RNA ligase. In some embodiments, the functional fragment will be truncated by less than 5, less than 10, less than 15, less than 10, less than 25, less than 30, less than 35, less than 40, less than 45, less than 50, less than 55, less than 60, less than 65, or less than 70 amino acids.

In some embodiments, the functional fragments or biologically active fragments of the engineered RNA ligase polypeptide described herein include at least a mutation or mutation set in the amino acid sequence of the engineered RNA ligase described herein. Accordingly, in some embodiments, the functional fragments or biologically active fragments of the engineered RNA ligase displays the enhanced or improved property associated with the mutation or mutation set in the parent RNA ligase.

### Polynucleotides Encoding Engineered Polypeptides, Expression Vectors and Host Cells

In another aspect, the present disclosure provides recombinant polynucleotides encoding the engineered RNA ligase described herein. In some embodiments, the recombinant polynucleotides are operably linked to one or more heterologous regulatory sequences that control gene expression to create a recombinant polynucleotide construct capable of expressing the engineered RNA ligase. In some embodiments, expression constructs containing at least one heterologous polynucleotide encoding the engineered RNA ligase polypeptide(s) is introduced into appropriate host cells to express the corresponding RNA ligase polypeptide(s).

As will be apparent to the skilled artisan, availability of a protein sequence and the knowledge of the codons corresponding to the various amino acids provide a description of all the polynucleotides capable of encoding the subject polypeptides. The degeneracy of the genetic code, where the same amino acids are encoded by alternative or synonymous codons, allows an extremely large number of nucleic acids to be made, all of which encode an engineered RNA ligase of the present disclosure. Thus, the present disclosure provides methods and compositions for the production of each and every possible variation of polynucleotides that could be made that encode the engineered RNA ligase polypeptides described herein by selecting combinations based on the possible codon choices, and all such variations of polynucleotides are to be considered specifically disclosed for any polypeptide described herein, including the amino acid sequences presented in the Examples (e.g., in Tables 5.1, 6.1, 7.1, 8.1, 9.1, 10.1, 11.1, and 12.1) and in the accompanying Sequence Listing.

In some embodiments, the codons are preferably optimized for utilization by the chosen host cell for protein production. In some embodiments, preferred codons in bacterial cells are used for expression in bacterial cells. In some embodiments, preferred codons in fungal cells are used for expression in fungal cells. In some embodiments, preferred codons in insect cells are used for expression in insect cells. In some embodiments, preferred codons in mammalian cells are used for expression in mammalian cells. In some embodiments, codon optimized polynucleotides encoding an engineered RNA ligase polypeptide described herein contain preferred codons at about 40%, 50%, 60%, 70%, 80%, 90%, or greater than 90% of the codon positions in the full length coding region.

Accordingly, in some embodiments, a recombinant polynucleotide of the present disclosure encodes an engineered RNA ligase polypeptides described herein. In some embodiments, the polynucleotide sequence of the recombinant polynucleotide is codon optimized.

In some embodiments, the recombinant polynucleotide comprises a polynucleotide sequence encoding an engineered RNA ligase comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 346 of an even-numbered SEQ ID NO. of SEQ ID NOs: 14 and 24-582, or to a reference sequence corresponding to an even-numbered SEQ ID NO. of SEQ ID NOs: 14 and 24-582, wherein the amino acid sequence comprises one or more substitutions relative to the reference sequence corresponding to residues 12 to 346 of SEQ ID NO: 14, 42, or 204, or relative to the reference sequence corresponding to SEQ ID NO: 14, 42, or 204.

In some embodiments, the recombinant polynucleotide comprises a polynucleotide sequence encoding an engineered RNA ligase comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 14, 42, or 204, or to the reference sequence corresponding to SEQ ID NO: 14, 42, or 204, wherein the amino acid sequence comprises one or more substitutions relative to the reference sequence corresponding to residues 12 to 346 of SEQ ID NO: 14, 42, or 204, or relative to the reference sequence corresponding to SEQ ID NO: 14, 42, or 204.

In some embodiments, the recombinant polynucleotide comprises a polynucleotide sequence encoding an engineered RNA ligase comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 14, or to the reference sequence corresponding to SEQ ID NO: 14, wherein the amino acid sequence comprises one or more substitutions relative to the reference sequence corresponding to residues 12 to 346 of SEQ ID NO: 14, or relative to the reference sequence corresponding to SEQ ID NO: 14.

In some embodiments, the recombinant polynucleotide comprises a polynucleotide sequence encoding an engineered RNA ligase comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 42 or 204, or to the reference sequence corresponding to SEQ ID NO: 42 or 204, wherein the amino acid sequence comprises one or more substitutions relative to the reference sequence corresponding to residues 12 to 346 of SEQ ID NO: 14, or relative to the reference sequence corresponding to SEQ ID NO: 14.

In some embodiments, the recombinant polynucleotide encodes an engineered RNA ligase comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or more sequence identity to the reference sequence corresponding to residues 12 to 346 of an even numbered SEQ ID NO. of SEQ ID NOs: 24-582, or to the reference sequence corresponding to an even numbered SEQ ID NO. of SEQ ID NOs: 24-582, wherein the amino acid sequence comprises one or more substitutions relative to the reference sequence corresponding to residues 12 to 346 of SEQ ID NO: 14, or relative to the reference sequence corresponding to SEQ ID NO: 14.

In some embodiments, the recombinant polynucleotide encodes an engineered RNA ligase comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 24-128, or to the reference sequence corresponding to an even-numbered SEQ ID NO. of SEQ ID NOs: 24-128, wherein the amino acid sequence comprises one or more substitutions relative to the reference sequence corresponding to residues 12 to 346 of SEQ ID NO: 14, or relative to the reference sequence corresponding to SEQ ID NO: 14.

In some embodiments, the recombinant polynucleotide comprises a polynucleotide sequence encoding an engineered RNA ligase comprising an amino acid sequence comprising at least a substitution at amino acid position 2, 14, 15, 18, 33, 39, 63, 69, 81, 83, 85, 86, 89, 95, 116, 117, 119, 138, 142, 144, 149, 154, 156, 159, 171, 175, 177, 181, 185, 186, 195, 202, 212, 214, 224, 226, 230, 247, 256, 257, 260, 266, 275, 280, 283, 285, 288, 291, 296, 303, 306, 307, 310, 314, 315, 316, 317, 326, 330, 331, 333, 334, 335, 337, 339, 342, or 345, or combinations thereof, wherein the amino acid positions are relative to the reference sequence corresponding to residues 12 to 346 of SEQ ID NO: 14, or relative to the reference sequence corresponding to SEQ ID NO: 14.

In some embodiments, the recombinant polynucleotide comprises a polynucleotide sequence encoding an engineered RNA ligase comprising an amino acid sequence comprising at least a substitution at amino acid position 95 or 177, or combination thereof, wherein the amino acid positions are relative to the reference sequence corresponding to residues 12 to 346 of SEQ ID NO: 14, or relative to the reference sequence corresponding to SEQ ID NO: 14.

In some embodiments, the recombinant polynucleotide comprises a polynucleotide sequence encoding an engineered RNA ligase comprising an amino acid sequence comprising at least a substitution at amino acid position 15, 95, 149, 154, 156, 177, 186, 195, 224, or 226, or combinations thereof, wherein the amino acid positions are relative to the reference sequence corresponding to residues 12 to 346 of SEQ ID NO: 14, or relative to the reference sequence corresponding to SEQ ID NO: 14.

In some embodiments, the recombinant polynucleotide comprises a polynucleotide sequence encoding an engineered RNA ligase comprising an amino acid sequence comprising at least a substitution or substitution set at amino acid position(s) 177/260/307/345, 171/175/183/280/283/303, 95/159/177/260/288, 175/183/247/280/283/296, 177/307, 81/171/183/335, 175/183, 81/175/183/280/296/335/339, 81/183/247/266, 95/177, 171/280/283/296/303, 85/177/260/288/333/345, 177/291/307/333, 81/171/175/183/316/337, 81/171/266/280/283/335/339, 177, 95/159/177/260/280/288/306/345, 171/175/335, 177/260/30/333, 14/95/177/288/307, 95/177/345, 95/159/177/260/285/288, 266/296, 81/283/335/337, 95/159/177/260/342/345, 63/171/175/183/266/280/296/316, 280/335/337, 81/171/280, 81/171/175/296/316, 171/175/316/335, 171/280/283/296/316, 171/283/335, 63/171/175/183/266/283/303/316, 171/247/266/283/296/303/316/335, 63/81/171/175/183/266/280/283/337, 81/171/303/316/335, 288/307, 14/95/159/177/345, 81/171/175/183/247/266/280/283, 81/247/266, 280, 85/177/260, 63/183, 175/183/247/280/283/316, 291, 247, 171/280/283, 81/171/175/280/316, 333, 171/296/316, 81/171, or 280/316/335, wherein the amino acid positions are relative to the reference sequence corresponding to residues 12 to 346 of SEQ ID NO: 14, or relative to the reference sequence corresponding to SEQ ID NO: 14.

In some embodiments, the recombinant polynucleotide comprises a polynucleotide sequence encoding an engineered RNA ligase comprising an amino acid sequence comprising at least a substitution at an amino acid position provided in Tables 5.1, 6.1, 7.1, 8.1, 9.1, 10.1, 11.1, and 12.1, wherein the amino acid positions are relative to the reference sequence corresponding to residues 12 to 346 of SEQ ID NO: 14, or relative to the reference sequence corresponding to SEQ ID NO: 14.

In some embodiments, the recombinant polynucleotide comprises a polynucleotide sequence encoding an engineered RNA ligase comprising an amino acid sequence comprising at least one substitution provided in Tables 5.1, 6.1, 7.1, 8.1, 9.1, 10.1, 11.1, and 12.1, wherein the amino acid positions are relative to the reference sequence corresponding to residues 12 to 346 of SEQ ID NO: 14, or relative to the reference sequence corresponding to SEQ ID NO: 14.

In some embodiments, the recombinant polynucleotide comprises a polynucleotide sequence encoding an engineered RNA ligase comprising an amino acid sequence comprising at least a substitution or substitution set at the amino acid position(s) set forth in Tables 5.1, 6.1, 7.1, 8.1, 9.1, 10.1, 11.1, and 12.1, wherein the amino acid positions are relative to the reference sequence corresponding to residues 12 to 346 of SEQ ID NO: 14, or relative to the reference sequence corresponding to SEQ ID NO: 14.

In some embodiments, the recombinant polynucleotide comprises a polynucleotide sequence encoding an engineered RNA ligase comprising an amino acid sequence comprising at least a substitution or substitution set of an RNA ligase variant set forth in Tables 5.1, 6.1, 7.1, 8.1, 9.1, 10.1, 11.1, and 12.1, wherein the amino acid positions are relative to the reference sequence corresponding to residues 12 to 346 of SEQ ID NO: 14, or relative to the reference sequence corresponding to SEQ ID NO: 14.

In some embodiments, the recombinant polynucleotide comprises a polynucleotide sequence encoding an engineered RNA ligase comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 42 or 204, or to the reference sequence corresponding to SEQ ID NO: 42 or 204.

In some embodiments, the recombinant polynucleotide comprises a polynucleotide sequence encoding an engineered RNA ligase comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 24-582, or to the reference sequence corresponding to an even-numbered SEQ ID NO. of SEQ ID NOs: 24-582.

In some embodiments, the recombinant polynucleotide comprises a polynucleotide sequence encoding an engineered RNA ligase comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 42 or 204, or to the reference sequence corresponding to SEQ ID NO: 42 or 204, wherein the amino acid sequence comprises one or more substitutions relative to the reference sequence corresponding to residues 12 to 346 of SEQ ID NO: 42 or 204, or relative to the reference sequence corresponding to SEQ ID NO: 42 or 204.

In some embodiments, the recombinant polynucleotide comprises a polynucleotide sequence encoding an engineered RNA ligase comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even numbered SEQ ID NO. of SEQ ID NOs: 24-582, or to the reference sequence corresponding to an even numbered SEQ ID NO. of SEQ ID NOs: 24-582, wherein the amino acid sequence comprises one or more substitutions relative to the reference sequence corresponding to residues 12 to 346 of SEQ ID NO: 42 or 204, or relative to the reference sequence corresponding to SEQ ID NO: 42 or 204.

In some embodiments, the recombinant polynucleotide comprises a polynucleotide sequence encoding an engineered RNA ligase comprising an amino acid sequence comprising at least a substitution at amino acid position 2, 14, 15, 18, 33, 39, 63, 69, 81, 83, 85, 86, 89, 95, 116, 117, 119, 138, 142, 144, 149, 154, 156, 159, 171, 175, 177, 181, 185, 186, 195, 202, 212, 214, 224, 226, 230, 247, 256, 257, 260, 266, 275, 280, 283, 285, 288, 291, 296, 303, 306, 307, 310, 314, 315, 316, 317, 326, 330, 331, 333, 334, 335, 337, 339, 342, or 345, or combinations thereof, wherein the amino acid positions are relative to the reference sequence corresponding to residues 12 to 346 of SEQ ID NO: 42 or 204, or relative to the reference sequence corresponding to SEQ ID NO: 42 or 204.

In some embodiments, the recombinant polynucleotide comprises a polynucleotide sequence encoding an engineered RNA ligase comprising an amino acid sequence comprising at least a substitution at amino acid position 95 or 177, or combination thereof, wherein the amino acid positions are relative to the reference sequence corresponding to residues 12 to 346 of SEQ ID NO: 42 or 204, or relative to the reference sequence corresponding to SEQ ID NO: 42 or 204.

In some embodiments, the recombinant polynucleotide comprises a polynucleotide sequence encoding an engineered RNA ligase comprising an amino acid sequence comprising at least a substitution at amino acid position 15, 95, 149, 154, 156, 177, 186, 195, 224, or 226, or combinations thereof, wherein the amino acid positions are relative to the reference sequence corresponding to residues 12 to 346 of SEQ ID NO: 42 or 204, or relative to the reference sequence corresponding to SEQ ID NO: 42 or 204.

In some embodiments, the recombinant polynucleotide comprises a polynucleotide sequence encoding an engineered RNA ligase comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 42, or to the reference sequence corresponding to SEQ ID NO: 42, wherein the amino acid sequence comprises one or more substitutions relative to the reference sequence corresponding to residues 12 to 346 of SEQ ID NO: 42, or relative to the reference sequence corresponding to SEQ ID NO: 42.

In some embodiments, the recombinant polynucleotide comprises a polynucleotide sequence encoding an engineered RNA ligase comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 130-478, or to the reference sequence corresponding to an even-numbered SEQ ID NO. of SEQ ID NOs: 130-478, wherein the amino acid sequence comprises one or more substitutions relative to the reference sequence corresponding to residues 12 to 346 of SEQ ID NO: 42, or relative to the reference sequence corresponding to SEQ ID NO: 42.

In some embodiments, the recombinant polynucleotide comprises a polynucleotide sequence encoding an engineered RNA ligase comprising an amino acid sequence comprising at least a substitution or substitution set at amino acid position(s) 149/156/195, 15/149/186/224/317, 15/156/195/224/226/317/331, 15/195/317/331, 149/224/331, 15/195/224/226/317/331, 15/33/86/149/154/195/317, 15/86/154/156/186/195/224/226/331, 15/149/224/317/331, 15/154/156/186/317/331, 15/33/149/224/226/331, 15/186/224/226/317/331, 15/33/149/186/224/331, 195/224/331, 15/86/156/186/195/226/317/331, 15/195/22/331, 156/186/224/226/331, 15/149/154/156/224/226, 33/156/186/195/331, 15/86/149/154/156/317/331, 15/186/317/331, 15/86/149/154/195/331, 15/33/149/154/156/226, 15/149/154/186/317/331, 195/331, 15/86/156/186/195/331, 149/156/224/226/331, 15/149/154/156/186/317/331, 15/154/186/195, 15/86/195/224/226, 230, 15/33/156/331, 15/33/156/195/224/226, 33/149/156/317, 15/33/186/195, 334, 15/154/156/224/317, 15/149/186/331, 15/86/149/186/195/317, 15/33/154/195, 15/156/186/331, 15/149/186/317, 15/33/156/186/214/224/331, 15/86/186/224/226/317, 15/154/331, 15/149/154/226, 15/33/195/226, 15/224/317/331, 15/86/186/195/317, 226/317/331, 330, 89, 15/149/186, 156/195/331, 156, 15/86/156/186/331, 275, 15/33/186/224, 15/186/317, 15/33/186/226/317, 15/149/154/156/331, 15/154/226/317, 15/86/156/186/195/317, 257, 15/154/156/186, 33/186/224, 335, 15/86/156/186/195/224/331, 154/156/317, 39, 2, 15/224/226/331, 15/33/149/224, 89/307, 15/154/156/317, 15/186/331, 15/33/154/317, 15/33/195/224/317, 15/156/224/226, 154/186/331, 185, 15/18/149/186/195, 86/149/154/156, 15/33/149/331, 326, 202, 119, 212, 15/33/156/317, 280, 142, 15/149, 15/33/86/156/195, 315, 256, 15/33/156/186, 138, 117, 116, 316, 69, 310, 317, 15/195/331, 15/33/331, 314, or 144, wherein the amino acid positions are relative to the reference sequence corresponding to residues 12 to 346 of SEQ ID NO: 42, or relative to the reference sequence corresponding to SEQ ID NO: 42.

In some embodiments, the recombinant polynucleotide comprises a polynucleotide sequence encoding an engineered RNA ligase comprising an amino acid sequence comprising at least a substitution or substitution set at amino acid position(s) 15/33/149/154/186/195, 149/156/186/195/224/226/317, 15/149/156/195/224/226, 15/33/86/149/154/156/186/195/226/331, 15/149/156/186/195/224/317/331, 15/33/149/154/195/317, 15/149/154/195/224/317, 15/149/156/186/224/226/317/331, 15/33/149/154/156/186/195/224/226/317/331, 149/195/224, 86/149/154/156/186/195/331, 15/33/149/156/195/224/317/331, 15/149/154/156/186/195/317/331, 15/33/149/154/156/186/224/331, 15/149/154/156/195/224, 15/149/154/156/195/224/226, 15/149/154/156/186/195/224/317, 33/149/156/186/224/331, 15/33/86/149/195/224/331, 15/86/149/154/156/186/195/331, 149/186/195/331, 15/154/156/186/226/331, 15/149/154/186/195/224/226, 15/149/154/156/195/224/331, 15/33/149/154/156/195/317/331, 15/33/154/156/186/195/224/317/331, 15/149/186/195/224/331, 15/33/149/154/156/195/224/331, 15/149/154/156/224/226/317/331, 15/33/149/156/195/331, 15/149/154/156/186/224/226/331, 154/156/195/224/226, 15/33/86/149/154/156/186/195/224/317/331, 15/149/154/156/186/195/224/226, 15/33/149/195/317/331, 15/149/154/224/331, 149/154/156/195/224/226/331, 15/149/154/195/331, 15/149/154/156/186/195, 15/33/149/154/156/186/195, 15/33/86/149/154/195/224/317/331, or 149/186/195/224/226/331, wherein the amino acid positions are relative to the reference sequence corresponding to residues 12 to 346 of SEQ ID NO: 42, or relative to the reference sequence corresponding to SEQ ID NO: 42.

In some embodiments, the recombinant polynucleotide comprises a polynucleotide sequence encoding an engineered RNA ligase comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 204, or to the reference sequence corresponding to SEQ ID NO: 204, wherein the amino acid sequence comprises one or more substitutions relative to the reference sequence corresponding to residues 12 to 346 of SEQ ID NO: 204, or relative to the reference sequence corresponding to SEQ ID NO: 204.

In some embodiments, the recombinant polynucleotide comprises a polynucleotide sequence encoding an engineered RNA ligase comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 480-582, to the reference sequence corresponding to an even-numbered SEQ ID NO. of SEQ ID NOs: 480-582, wherein the amino acid sequence comprises one or more substitutions relative to the reference sequence corresponding to residues 12 to 346 of SEQ ID NO: 204, or relative to the reference sequence corresponding to SEQ ID NO: 204.

In some embodiments, the recombinant polynucleotide comprises a polynucleotide sequence encoding an engineered RNA ligase comprising an amino acid sequence comprising at least a substitution or substitution set at amino acid position(s) 2/39/69/144/156/316/330, 2/39/69/119/185/316, 69/212/256/330, 39/119/256/316/326/330, 69/116/119/185/330, 2/39/119/144/156/159/202/256/316/326/330, 81/183/185/230, 2/39/69/116/144/156/185, 2/14/39/69/116/119/159/185/256/326, 2/116/119/316, 116/119/144/185/314/316, 39/69/116/144/181/185/256/330, 2/39/69/119/144/156/185/314, 14/39/69/144/156/212/256/314/316/326/330, 39/69/144/156/185/316/330, 156/185/334/335, 39/63/156/230/257/275/330/335, 156/266/316/330/334/335, 39/63/156/183/185/230/330/334, 69/144/316, 116/119/156/202/212/326/330, 39/69/119/138/181/185/316/326, 39/81/156/230, 2/14/69/116/119/144/159/326/330, 39/156/334/345, 230/266/334, 2/116/144/156/159, 156/183/185/230, 2/14/116/316/326/330, 39/63/156/230/316/345, 69/138/144/159/185/202/316, 39/156/257/266/316/334, 156/257/275/334, 183, 89/156/185/230/316/345, 2/14/119, 144/156/316/330, 39/81/185/316/334, 89/230/266/345, 39/156/230/275/316, 39/156/230/266/334/335, 156/185/275/316/330, 119/156/202, 230/257/275/316/330/345, 230, 81/156/330/335/345, 156/183/230/266, 39/185, 39/81/183/275/316/334/345, 156/230/257/316, 39/116/156, or 183/185/257/275/316/330/334, wherein the amino acid positions are relative to the reference sequence corresponding to residues 12 to 346 of SEQ ID NO: 204, or relative to the reference sequence corresponding to SEQ ID NO: 204.

In some embodiments, the recombinant polynucleotide comprises a polynucleotide sequence encoding an engineered RNA ligase comprising an amino acid sequence comprising a substitution at an amino acid position provided in Tables 6.1, 7.1, 8.1, 9.1, 10.1, 11.1, and 12.1, wherein the amino acid positions are relative to the reference sequence corresponding to residues 12 to 346 of SEQ ID NO: 42 or 204, or relative to the reference sequence corresponding to SEQ ID NO: 42 or 204, as provided in each of the Tables.

In some embodiments, the recombinant polynucleotide comprises a polynucleotide sequence encoding an engineered RNA ligase comprising an amino acid sequence comprising at least one substitution provided in Tables 6.1, 7.1, 8.1, 9.1, 10.1, 11.1, and 12.1, wherein the amino acid positions are relative to the reference sequence corresponding to residues 12 to 346 of SEQ ID NO: 42 or 204, or relative to the reference sequence corresponding to SEQ ID NO: 42 or 204, as provided in each of the Tables.

In some embodiments, the recombinant polynucleotide comprises a polynucleotide sequence encoding an engineered RNA ligase comprising at least a substitution or substitution set at amino acid position(s) provided in Tables 6.1, 7.1, 8.1, 9.1, 10.1, 11.1, and 12.1, wherein the amino acid positions are relative to the reference sequence corresponding to residues 12 to 346 of SEQ ID NO: 42 or 204, or relative to the reference sequence corresponding to SEQ ID NO: 42 or 204, as provided in each of the Tables.

In some embodiments, the recombinant polynucleotide comprises a polynucleotide sequence encoding an engineered RNA ligase comprising at least a substitution or substitution set of an RNA ligase variant provided in Tables 6.1, 7.1, 8.1, 9.1, 10.1, 11.1, and 12.1, wherein the amino acid positions are relative to the reference sequence corresponding to residues 12 to 346 of SEQ ID NO: 42 or 204, or relative to the reference sequence corresponding to SEQ ID NO: 42 or 204, as provided in each of the Tables.

In some embodiments, the recombinant polynucleotide comprises a polynucleotide sequence encoding an engineered RNA ligase comprising an amino acid sequence having least 80%, 81%, 82%, 83%, 84%, 85%, 86%, 87%, 88%, 89%, 90%, 91%, 92%, 93%, 94%, 95%, 96%, 97%, 98%, 99% or more sequence identity to a reference sequence comprising a substitution or substitution set provided in Tables 5.1, 6.1, 7.1, 8.1, 9.1, 10.1, 11.1, and 12.1, wherein the amino acid positions are relative to the reference sequence corresponding to residues 12 to 346 of SEQ ID NO: 14, 42, or 204, or relative to the reference sequence corresponding to SEQ ID NO: 14, 42, or 204.

In some embodiments, the recombinant polynucleotide comprises a polynucleotide sequence encoding an engineered RNA ligase comprising an amino acid sequence having least 80%, 81%, 82%, 83%, 84%, 85%, 86%, 87%, 88%, 89%, 90%, 91%, 92%, 93%, 94%, 95%, 96%, 97%, 98%, 99% or more sequence identity to the sequence comprising residues 12 to 346 of an engineered RNA ligase set forth in Tables 5.1, 6.1, 7.1, 8.1, 9.1, 10.1, 11.1, and 12.1, or to the sequence of an engineered RNA ligase set forth in Tables 5.1, 6.1, 7.1, 8.1, 9.1, 10.1, 11.1, and 12.1.

In some embodiments, the recombinant polynucleotide comprises a polynucleotide sequence encoding an engineered RNA ligase comprising residues 12 to 346 of an even numbered SEQ ID NO. of SEQ ID NOs: 24-582, or an amino acid sequence comprising an even numbered SEQ ID NO. of SEQ ID NOs: 24-582, optionally wherein the amino acid sequence has 1, 2, 3, 4, 5, 6, 7, 8, 9, or up to 10 substitutions.

In some embodiments, the recombinant polynucleotide comprises a polynucleotide sequence encoding an engineered RNA ligase comprising residues 12 to 346 of SEQ ID NO: 42 or 204, or an amino acid sequence comprising SEQ ID NO: 42 or 204, optionally wherein the amino acid sequence has 1, 2, 3, 4, 5, 6, 7, 8, 9, or up to 10 substitutions in the amino acid sequence.

In some embodiments, the recombinant polynucleotide comprises a polynucleotide sequence having at least 70%, 75%, 80%, 81%, 82%, 83%, 84%, 85%, 90%, 91%, 92%, 93%, 94%, 95%, 96%, 97%, 98%, 99%, or more sequence identity to a reference polynucleotide sequence corresponding to nucleotide residues 34 to 1038 of an odd numbered SEQ ID NO. of SEQ ID NOs: 13-581, or to a reference polynucleotide sequence corresponding to an odd numbered SEQ ID NO. of SEQ ID NOs: 13-581, wherein the recombinant polynucleotide encodes an RNA ligase.

In some embodiments, the recombinant polynucleotide comprises a polynucleotide sequence having at least 70%, 75%, 80%, 81%, 82%, 83%, 84%, 85%, 90%, 91%, 92%, 93%, 94%, 95%, 96%, 97%, 98%, 99%, or more sequence identity to a reference polynucleotide sequence corresponding to nucleotide residues 34 to 1038 of SEQ ID NO: 13, 41, or 203, or to a reference polynucleotide sequence corresponding to SEQ ID NO: 13, 41, or 203, wherein the recombinant polynucleotide encodes an RNA ligase.

In some embodiments, the recombinant polynucleotide comprises a polynucleotide sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to the reference sequence corresponding to nucleotide residues 34 to 1038 of SEQ ID NO: 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 573, 575, 577, 579, or 581, wherein the recombinant polynucleotide encodes an RNA ligase.

In some embodiments, the recombinant polynucleotide comprises a polynucleotide sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to the reference sequence corresponding to SEQ ID NO: 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415,417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 573, 575, 577, 579, or 581, wherein the recombinant polynucleotide encodes an RNA ligase.

In some embodiments, the recombinant polynucleotide comprises a polynucleotide sequence comprising nucleotide residues 34 to 1038 of an odd numbered SEQ ID NO. of SEQ ID NOs: 13-581, or a polynucleotide sequence comprising an odd numbered SEQ ID NO. of SEQ ID NOs: 13-581.

In some embodiments, the recombinant polynucleotide comprises a polynucleotide sequence comprising nucleotide residues 34 to 1038 of SEQ ID NO: 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 573, 575, 577, 579, or 581.

In some embodiments, the recombinant polynucleotide comprises a polynucleotide sequence comprising SEQ ID NO: 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 573, 575, 577, 579, or 581.

In some embodiments, the recombinant polynucleotide comprises a polynucleotide sequence encoding an engineered RNA ligase comprising an amino acid sequence having at least 80%, 85%, 86%, 87%, 88%, 89%, 90%, 91%, 92%, 93%, 94%, 95%, 96%, 97%, 98%, 99% or more sequence identity to the sequence corresponding to:
residues 12 to 345 of SEQ ID NO: 2;
residues 12 to 343 of SEQ ID NO: 4;
residues 12 to 250 of SEQ ID NO: 6;
residues 12 to 346 of SEQ ID NO: 8;
residues 12 to 345 of SEQ ID NO: 10;
residues 12 to 345 of SEQ ID NO: 12;
residues 12 to 346 of SEQ ID NO: 14;
residues 12 to 346 of SEQ ID NO: 16;
residues 12 to 350 of SEQ ID NO: 18;
residues 12 to 350 of SEQ ID NO: 20; or
residues 12 to 344 of SEQ ID NO: 22.

In some embodiments, the recombinant polynucleotide comprises a polynucleotide sequence encoding an engineered RNA ligase comprising an amino acid sequence having at least 80%, 85%, 86%, 87%, 88%, 89%, 90%, 91%, 92%, 93%, 94%, 95%, 96%, 97%, 98%, 99% or more sequence identity to the sequence corresponding to SEQ ID NO: 2, 4, 6, 8, 10, 12, 14, 16, 18, 20 or 22.

In some embodiments, the recombinant polynucleotide comprises a polynucleotide sequence encoding an engineered RNA ligase comprising an amino acid sequence comprising:
residues 12 to 345 of SEQ ID NO: 2;
residues 12 to 343 of SEQ ID NO: 4;
residues 12 to 250 of SEQ ID NO: 6;
residues 12 to 346 of SEQ ID NO: 8;
residues 12 to 345 of SEQ ID NO: 10;
residues 12 to 345 of SEQ ID NO: 12;
residues 12 to 346 of SEQ ID NO: 14;
residues 12 to 346 of SEQ ID NO: 16;
residues 12 to 350 of SEQ ID NO: 18;
residues 12 to 350 of SEQ ID NO: 20; or
residues 12 to 344 of SEQ ID NO: 22.

In some embodiments, the recombinant polynucleotide comprises a polynucleotide sequence encoding an engineered RNA ligase comprising an amino acid sequence comprising SEQ ID NO: 2, 4, 6, 8, 10, 12, 14, 16, 18, 20 or 22.

In some embodiments, the present disclosure provides a recombinant polynucleotide capable of hybridizing under highly stringent conditions to a reference polynucleotide encoding an engineered RNA ligase polypeptide described herein, e.g., a recombinant polynucleotide provided in Tables 5.1, 6.1, 7.1, 8.1, 9.1, 10.1, 11.1, and 12.1, or a reverse complement thereof. In some embodiments, the recombinant polynucleotide hybridizes under highly stringent conditions to a reference polynucleotide corresponding to nucleotide residues 34 to 1038 of SEQ ID NO: 13, 41, or 203, or to a reference polynucleotide sequence corresponding to SEQ ID NO: 13, 41, or 203, or a reverse complement thereof. In some embodiments, the recombinant polynucleotide hybridizes under highly stringent conditions to a polynucleotide sequence corresponding to nucleotide residues 34 to 1038 of an odd numbered SEQ ID NO. of SEQ ID NOs: 23-581, or a polynucleotide sequence comprising an odd numbered SEQ ID NO. of SEQ ID NOs: 23-581, or a reverse complement thereof.

In some embodiments, the present disclosure provides a recombinant polynucleotide capable of hybridizing under highly stringent conditions to a reverse complement of a reference polynucleotide encoding an engineered RNA ligase polypeptide described herein, wherein the recombinant polynucleotide hybridizing under stringent conditions encodes an RNA ligase polypeptide comprising an amino acid sequence having one or more amino acid differences as compared to SEQ ID NO: 14, 42, or 204, at residue positions selected from any positions as set forth in Tables 5.1, 6.1, 7.1, 8.1, 9.1, 10.1, 11.1, and 12.1. In some embodiments, the recombinant polynucleotide that hybridizes under highly stringent conditions to a reverse complement of a reference polynucleotide encoding an engineered RNA ligase polypeptide described herein encodes an RNA ligase polypeptide having one or more amino acid differences present in an engineered RNA ligase having an amino acid sequence corresponding to residues 12 to 346 of an even numbered SEQ ID NO. of SEQ ID NOs: 24-582, or an amino acid sequence comprising an even numbered SEQ ID NO. of SEQ ID NOs: 24-582, wherein the amino acid differences are relative to SEQ ID NO: 14, 42, or 204.

In some embodiments, the recombinant polynucleotide that hybridizes under highly stringent conditions comprises a polynucleotide sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a reference polynucleotide sequence corresponding to nucleotide residues 34 to 1038 of SEQ ID NO: 23, 41, or 203, or to a reference polynucleotide sequence corresponding to SEQ ID NO: 23, 41, or 203, or a reverse complement thereof. In some embodiments, the recombinant polynucleotide that hybridizes under highly stringent conditions comprises a polynucleotide sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a reference polynucleotide sequence corresponding to nucleotide residues 34 to 1038 of an odd-numbered SEQ ID NO. of SEQ ID NOs: 23-581, or an odd-numbered SEQ ID NO. of SEQ ID NOs: 23-581, or a reverse complement thereof.

In some additional embodiments, the polynucleotide hybridizing under highly stringent conditions comprises a polynucleotide sequence having at least 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a reverse complement of a polynucleotide reference sequence corresponding to nucleotide residues 34 to 1038 of SEQ ID NO: 23, 41, or 203, or to a reference polynucleotide sequence corresponding to SEQ ID NO: 23, 41, or 203 encodes an engineered RNA ligase polypeptide. In some embodiments, the polynucleotide hybridizing under highly stringent conditions comprises a polynucleotide sequence having at least 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a reverse complement of a polynucleotide reference sequence corresponding to nucleotide residues 34 to 1038 of an odd-numbered SEQ ID NO. of SEQ ID NOs: 23-581, or an odd-numbered SEQ ID NO. of SEQ ID NOs: 23-581 encodes an engineered RNA ligase polypeptide.

In some embodiments, a recombinant polynucleotide encoding any of the RNA ligases herein is manipulated in a variety of ways to facilitate expression of the RNA ligase polypeptide. In some embodiments, the recombinant polynucleotide encoding the RNA ligase comprises expression vectors where one or more control sequences is present to regulate the expression of the RNA ligase polynucleotides and/or polypeptides. Manipulation of the isolated polynucleotide prior to its insertion into a vector may be desirable or necessary depending on the expression vector utilized. Techniques for modifying polynucleotides and nucleic acid sequences utilizing recombinant DNA methods are well known in the art. In some embodiments, the control sequences include among others, promoters, leader sequences, polyadenylation sequences, propeptide sequences, signal peptide sequences, and transcription terminators.

In some embodiments, suitable promoters are selected based on the host cell selection. For bacterial host cells, suitable promoters for directing transcription of the nucleic acid constructs of the present disclosure, include, but are not limited to promoters obtained from the E. coli lac operon, Streptomyces coelicolor agarase gene (dagA), Bacillus subtilis levansucrase gene (sacB), Bacillus licheniformis alpha-amylase gene (amyL), Bacillus stearothermophilus maltogenic amylase gene (amyM), Bacillus amyloliquefaciens alpha-amylase gene (amyQ), Bacillus licheniformis penicillinase gene (penP), Bacillus subtilis xylA and xylB genes, and prokaryotic beta-lactamase gene (see, e.g., Villa-Kamaroff et al., Proc. Natl Acad. Sci. USA, 1978, 75:3727-3731), as well as the tac promoter (see, e.g., DeBoer et al., Proc. Natl Acad. Sci. USA, 1983, 80:21-25). Exemplary promoters for filamentous fungal host cells, include, but are not limited to promoters obtained from the genes for Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase, and Fusarium oxysporum trypsin-like protease (see, e.g., WO 96/00787), as well as the NA2-tpi promoter (a hybrid of the promoters from the genes for Aspergillus niger neutral alpha-amylase and Aspergillus oryzae triose phosphate isomerase), and mutant, truncated, and hybrid promoters thereof. Exemplary yeast cell promoters can be from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH2/GAP), and Saccharomyces cerevisiae 3-phosphoglycerate kinase. Other useful promoters for yeast host cells are known in the art (see, e.g., Romanos et al., Yeast, 1992, 8:423-488). Exemplary promoters for use in insect cells include, but are not limited to, polyhedrin, p10, ELT, OpIE2, and hr5/ie1 promoters. Exemplary promoters for use in mammalian cells include, but are not limited to, those from cytomegalovirus (CMV), chicken β-actin promoter fused with the CMV enhancer, Simian vacuolating virus 40 (SV40), from Homo sapiens phosphoglycerate kinase, beta actin, elongation factor-1a or glyceraldehyde-3-phosphate dehydrogenase, and from Gallus β-actin.

In some embodiments, the control sequence is a suitable transcription terminator sequence (i.e., a sequence recognized by a host cell to terminate transcription). In some embodiments, the terminator sequence is operably linked to the 3′ terminus of the nucleic acid sequence encoding the RNA ligase polypeptide. Any suitable terminator which is functional in the host cell of choice finds use in the present invention. For bacterial expression, the transcription terminators can be a Rho-dependent terminators that rely on a Rho transcription factor, or a Rho-independent, or intrinsic terminators, which do not require a transcription factor. Exemplary bacterial transcription terminators are described in Peters et al., J Mol Biol., 2011, 412(5):793-813. Exemplary transcription terminators for filamentous fungal host cells can be obtained from the genes for Aspergillus oryzae TAKA amylase, Aspergillus niger glucoamylase, Aspergillus nidulans anthranilate synthase, Aspergillus niger alpha-glucosidase, and Fusarium oxysporum trypsin-like protease. Exemplary terminators for yeast host cells can be obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are known in the art (see, e.g., Romanos et al., Yeast, 1992, 8(6):423-88). Exemplary terminators for mammalian cells include, but are not limited to those from cytomegalovirus (CMV), Simian virus 40 (SV40), from Homo sapiens growth hormone hGH, from bovine growth hormone BGH, and from human or rabbit beta globulin.

In some embodiments, the control sequence is a suitable leader sequence, a non-translated region of an mRNA that is used for translation by the host cell. In some embodiments, the leader sequence is operably linked to the 5′ terminus of the nucleic acid sequence encoding the RNA ligase polypeptide. Any suitable leader sequence that is functional in the host cell of choice find use in the present invention. Exemplary leaders for filamentous fungal host cells are obtained from the genes for Aspergillus oryzae TAKA amylase, and Aspergillus nidulans triose phosphate isomerase. Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP). Suitable leaders for mammalian host cells include but are not limited to the 5′-UTR element present in orthopoxvirus mRNA.

In some embodiments, the control sequence is a polyadenylation sequence (i.e., a sequence operably linked to the 3′ terminus of the nucleic acid sequence and which, when transcribed, is recognized by the host cell as a signal to add polyadenosine residues to transcribed mRNA). Any suitable polyadenylation sequence which is functional in the host cell of choice finds use in the present invention. Exemplary polyadenylation sequences for filamentous fungal host cells include, but are not limited to the genes for Aspergillus oryzae TAKA amylase, Aspergillus niger glucoamylase, Aspergillus nidulans anthranilate synthase, Fusarium oxysporum trypsin-like protease, and Aspergillus niger alpha-glucosidase. Useful polyadenylation sequences for yeast host cells are known (see, e.g., Guo and Sherman, Mol. Cell. Biol., 1995, 15:5983-5990). Useful polyadenylation and 3′ UTR sequences for mammalian host cells include, but are not limited to, the 3′-UTRs of α- and β-globin mRNAs that harbor several sequence elements that increase the stability and translation of mRNA.

In some embodiments, the control sequence is also a signal peptide (i.e., a coding region that codes for an amino acid sequence linked to the amino terminus of a polypeptide and directs the encoded polypeptide into the cell's secretory pathway). In some embodiments, the 5′ end of the coding sequence of the nucleic acid sequence inherently contains a signal peptide coding region naturally linked in translation reading frame with the segment of the coding region that encodes the secreted polypeptide. Alternatively, in some embodiments, the 5′ end of the coding sequence contains a signal peptide coding region that is foreign to the coding sequence. Any suitable signal peptide coding region which directs the expressed polypeptide into the secretory pathway of a host cell of choice finds use for expression of the engineered polypeptide(s). Effective signal peptide coding regions for bacterial host cells are the signal peptide coding regions include, but are not limited to those obtained from the genes for Bacillus NC1B 11837 maltogenic amylase, Bacillus stearothermophilus alpha-amylase, Bacillus licheniformis subtilisin, Bacillus licheniformis beta-lactamase, Bacillus stearothermophilus neutral proteases (nprT, nprS, nprM), and Bacillus subtilis prsA. Further signal peptides are known in the art (see, e.g., Simonen and Palva, Microbiol. Rev., 1993, 57:109-137). In some embodiments, effective signal peptide coding regions for filamentous fungal host cells include, but are not limited to the signal peptide coding regions obtained from the genes for Aspergillus oryzae TAKA amylase, Aspergillus niger neutral amylase, Aspergillus niger glucoamylase, Rhizomucor miehei aspartic proteinase, Humicola insolens cellulase, and Humicola lanuginosa lipase. Useful signal peptides for yeast host cells include, but are not limited to those from the genes for Saccharomyces cerevisiae alpha-factor and Saccharomyces cerevisiae invertase. Useful signal peptides for insect and mammalian host cells include but are not limited to, those from the genes for immunoglobulin gamma (IgG) and the signal peptide in a human secreted protein, such as human beta-galactosidase polypeptide.

In some embodiments, the control sequence is a propeptide coding region that codes for an amino acid sequence positioned at the amino terminus of a polypeptide. The resultant polypeptide is referred to as a “proenzyme,” “propolypeptide,” or “zymogen.” A propolypeptide can be converted to a mature active polypeptide by catalytic or autocatalytic cleavage of the propeptide from the propolypeptide. The propeptide coding region may be obtained from any suitable source, including, but not limited to the genes for Bacillus subtilis alkaline protease (aprE), Bacillus subtilis neutral protease (nprT), Saccharomyces cerevisiae alpha-factor, Rhizomucor miehei aspartic proteinase, and Myceliophthora thermophila lactase (see, e.g., WO 95/33836). Where both signal peptide and propeptide regions are present at the amino terminus of a polypeptide, the propeptide region is positioned next to the amino terminus of a polypeptide and the signal peptide region is positioned next to the amino terminus of the propeptide region.

In some embodiments, regulatory sequences are also utilized. These sequences facilitate the regulation of the expression of the polypeptide relative to the growth of the host cell. Examples of regulatory systems are those that cause the expression of the gene to be turned on or off in response to a chemical or physical stimulus, including the presence of a regulatory compound. In prokaryotic host cells, suitable regulatory sequences include, but are not limited to the lac, tac, and trp operator systems. In yeast host cells, suitable regulatory systems include, but are not limited to the ADH2 system or GAL1 system. In filamentous fungi, suitable regulatory sequences include, but are not limited to the TAKA alpha-amylase promoter, Aspergillus niger glucoamylase promoter, and Aspergillus oryzae glucoamylase promoter.

In another aspect, the present disclosure provides a recombinant expression vector comprising a recombinant polynucleotide encoding an engineered RNA ligase polypeptide, and one or more expression regulating regions such as a promoter and a terminator, a replication origin, etc., depending on the type of hosts into which they are to be introduced. In some embodiments, the various nucleic acid and control sequences described herein are joined together (i.e., operably linked) to produce recombinant expression vectors which include one or more convenient restriction sites to allow for insertion or substitution of the nucleic acid sequence encoding the RNA ligase polypeptide at such sites. Alternatively, in some embodiments, the nucleic acid sequence of the present disclosure is expressed by inserting the nucleic acid sequence or a nucleic acid construct comprising the sequence into an appropriate vector for expression. In some embodiments involving the creation of the expression vector, the coding sequence is located in the vector so that the coding sequence is operably linked with the appropriate control sequences for expression.

The recombinant expression vector may be any suitable vector (e.g., a plasmid or virus), that can be conveniently subjected to recombinant DNA procedures and bring about the expression of the RNA ligase polynucleotide sequence. The choice of the vector typically depends on the compatibility of the vector with the host cell into which the vector is to be introduced. The vectors may be linear or closed circular plasmids.

In some embodiments, the expression vector is an autonomously replicating vector (i.e., a vector that exists as an extra-chromosomal entity, the replication of which is independent of chromosomal replication, such as a plasmid, an extra-chromosomal element, a minichromosome, or an artificial chromosome). The vector may contain any means for assuring self-replication. In some alternative embodiments, the vector is one in which, when introduced into the host cell, it is integrated into the genome and replicated together with the chromosome(s) into which it has been integrated. Furthermore, in some embodiments, a single vector or plasmid, or two or more vectors or plasmids which together contain the total DNA to be introduced into the genome of the host cell, and/or a transposon is utilized.

In some embodiment, the recombinant polynucleotides may be provided on a non-replicating expression vector or plasmid. In some embodiments, the non-replicating expression vector or plasmid can be based on viral vectors defective in replication (see, e.g., Travieso et al., npj Vaccines, 2022, Vol. 7, Article 75).

In some embodiments, the expression vector contains one or more selectable markers, which permit selection of transformed cells. A “selectable marker” is a gene, the product of which provides for biocide or viral resistance, resistance to heavy metals, prototrophy to auxotrophs, and the like. Examples of bacterial selectable markers include, but are not limited to the dal genes from Bacillus subtilis or Bacillus licheniformis, or markers, which confer antibiotic resistance such as ampicillin, kanamycin, chloramphenicol or tetracycline resistance. Suitable markers for yeast host cells include, but are not limited to ADE2, HIS3, LEU2, LYS2, MET3, TRP1, and URA3. Selectable markers for use in filamentous fungal host cells include, but are not limited to, amdS (acetamidase; e.g., from A. nidulans or A. orzyae), argB (ornithine carbamoyltransferases), bar (phosphinothricin acetyltransferase; e.g., from S. hygroscopicus), hph (hygromycin phosphotransferase), niaD (nitrate reductase), pyrG (orotidine-5′-phosphate decarboxylase; e.g., from A. nidulans or A. orzyae), sC (sulfate adenyltransferase), and trpC (anthranilate synthase), as well as equivalents thereof.

In another aspect, the present disclosure provides a host cell comprising a recombinant polynucleotide encoding at least one engineered RNA ligase polypeptide described herein, the polynucleotide(s) being operably linked to one or more control sequences for expression of the engineered RNA ligase enzyme(s) in the host cell. In some embodiments, the host cell comprises an expression vector comprising a polynucleotide encoding an engineered RNA ligase polypeptide described herein, where the polynucleotide is operably linked to one or more control sequences. Host cells suitable for use in expressing the polypeptides encoded by the expression vectors of the present invention are known in the art and include but are not limited to, bacterial cells, such as E. coli, B. subtilis, Vibrio fluvialis, Streptomyces and Salmonella typhimurium cells; fungal cells, such as yeast cells (e.g., Saccharomyces cerevisiae or Pichia pastoris (ATCC Accession No. 201178)); insect cells such as Drosophila S2 and Spodoptera Sf9 cells; animal cells such as CHO, COS, BHK, 293, and Bowes melanoma cells; and plant cells. Exemplary host cells also include various Escherichia coli strains (e.g., W3110 (ΔfhuA) and BL21).

In another aspect, the present disclosure provides a method of producing the engineered RNA ligase polypeptides, where the method comprises culturing a host cell capable of expressing a polynucleotide encoding the engineered RNA ligase polypeptide under conditions suitable for expression of the polypeptide such that the engineered RNA ligase is produced. In some embodiments, the method further comprises the step(s) of isolating the RNA ligase polypeptides from the culture and/or host cells.

In some embodiments, the method further comprises purifying the expressed RNA ligase polypeptide, as described herein.

In some embodiments, the RNA ligase polypeptide expressed in a host cell is recovered from the cells and/or the culture medium using any one or more of the known techniques for protein purification, including, among others, lysozyme or detergent treatment, sonication, filtration, salting-out, ultra-centrifugation, and chromatography, such as described herein.

Chromatographic techniques for isolation/purification of the RNA ligase polypeptides include, among others, reverse phase chromatography, high-performance liquid chromatography, ion-exchange chromatography, hydrophobic-interaction chromatography, size-exclusion chromatography, gel electrophoresis, and affinity chromatography. Conditions for purifying the RNA ligase depends, in part, on factors such as net charge, hydrophobicity, hydrophilicity, molecular weight, molecular shape, etc., and will be apparent to those having skill in the art. In some embodiments, affinity techniques may be used to isolate the RNA ligase. For affinity chromatography purification, an antibody that specifically binds RNA ligase polypeptide may be used. In some embodiments, an affinity tag, e.g., His-tag, can be introduced into the RNA ligase polypeptide for purposes of isolation/purification.

Appropriate culture media and growth conditions for host cells are known in the art. It is contemplated that any suitable method for introducing polynucleotides for expression of the RNA ligase polypeptides into cells will find use in the present invention. Suitable techniques include, but are not limited to electroporation, biolistic particle bombardment, liposome mediated transfection, calcium chloride transfection, and protoplast fusion.

In some embodiments, recombinant polypeptides (e.g., RNA ligase enzyme variants) can be produced using any suitable methods known the art. For example, there is a wide variety of different mutagenesis techniques known to those skilled in the art. In addition, mutagenesis kits are also available from many commercial molecular biology suppliers. Methods are available to make specific substitutions at defined amino acids (site-directed), specific or random mutations in a localized region of the gene (region-specific), or random mutagenesis over the entire gene (e.g., saturation mutagenesis). Numerous suitable methods are known to those in the art to generate enzyme variants, including but not limited to site-directed mutagenesis of single-stranded DNA or double-stranded DNA using PCR, cassette mutagenesis, gene synthesis, error-prone PCR, shuffling, and chemical saturation mutagenesis, or any other suitable method known in the art. Non-limiting examples of methods used for DNA and protein engineering are provided in the following patents: U.S. Pat. Nos. 6,117,679; 6,420,175; 6,376,246; 6,586,182; 7,747,391; 7,747,393; 7,783,428; and 8,383,346. After the variants are produced, they can be screened for any desired property (e.g., high or increased activity, or low or reduced activity, increased thermal activity, increased stability, increased substrate range, increased inhibitor resistance or tolerance, increased salt tolerance, and/or pH stability, etc.). Exemplary methods are provided in the Examples.

In some embodiments, the engineered RNA ligase polypeptides with the properties disclosed herein can be obtained by subjecting the polynucleotide encoding the naturally occurring or engineered RNA ligase polypeptide to a suitable mutagenesis and/or directed evolution methods known in the art, for example, as described herein. An exemplary directed evolution technique is mutagenesis and/or DNA shuffling (see, e.g., Stemmer, Proc. Natl. Acad. Sci. USA, 1994, 91:10747-10751; WO 95/22625; WO 97/0078; WO 97/35966; WO 98/27230; WO 00/42651; WO 01/75767 and U.S. Pat. No. 6,537,746). Other directed evolution procedures that can be used include, among others, staggered extension process (StEP), in vitro recombination (see, e.g., Zhao et al., Nat. Biotechnol., 1998, 16:258-261), mutagenic PCR (see, e.g., Caldwell et al., PCR Methods Appl., 1994, 3:S136-S140), and cassette mutagenesis (see, e.g., Black et al., Proc. Natl. Acad. Sci. USA, 1996, 93:3525-3529).

Mutagenesis and directed evolution methods can be applied to RNA ligase-encoding polynucleotides to generate variant libraries that can be expressed, screened, and assayed. Any suitable mutagenesis and directed evolution methods find use in the present disclosure and are known in the art (see, e.g., U.S. Pat. Nos. 5,605,793, 5,811,238, 5,830,721, 5,834,252, 5,837,458, 5,928,905, 6,096,548, 6,117,679, 6,132,970, 6,165,793, 6,180,406, 6,251,674, 6,265,201, 6,277,638, 6,287,861, 6,287,862, 6,291,242, 6,297,053, 6,303,344, 6,309,883, 6,319,713, 6,319,714, 6,323,030, 6,326,204, 6,335,160, 6,335,198, 6,344,356, 6,352,859, 6,355,484, 6,358,740, 6,358,742, 6,365,377, 6,365,408, 6,368,861, 6,372,497, 6,337,186, 6,376,246, 6,379,964, 6,387,702, 6,391,552, 6,391,640, 6,395,547, 6,406,855, 6,406,910, 6,413,745, 6,413,774, 6,420,175, 6,423,542, 6,426,224, 6,436,675, 6,444,468, 6,455,253, 6,479,652, 6,482,647, 6,483,011, 6,484,105, 6,489,146, 6,500,617, 6,500,639, 6,506,602, 6,506,603, 6,518,065, 6,519,065, 6,521,453, 6,528,311, 6,537,746, 6,573,098, 6,576,467, 6,579,678, 6,586,182, 6,602,986, 6,605,430, 6,613,514, 6,653,072, 6,686,515, 6,703,240, 6,716,631, 6,825,001, 6,902,922, 6,917,882, 6,946,296, 6,961,664, 6,995,017, 7,024,312, 7,058,515, 7,105,297, 7,148,054, 7,220,566, 7,288,375, 7,384,387, 7,421,347, 7,430,477, 7,462,469, 7,534,564, 7,620,500, 7,620,502, 7,629,170, 7,702,464, 7,747,391, 7,747,393, 7,751,986, 7,776,598, 7,783,428, 7,795,030, 7,853,410, 7,868,138, 7,783,428, 7,873,477, 7,873,499, 7,904,249, 7,957,912, 7,981,614, 8,014,961, 8,029,988, 8,048,674, 8,058,001, 8,076,138, 8,108,150, 8,170,806, 8,224,580, 8,377,681, 8,383,346, 8,457,903, 8,504,498, 8,589,085, 8,762,066, 8,768,871, 9,593,326, 9,665,694, 9,684,771, and all related PCT and non-US counterparts; Ling et al., Anal. Biochem., 1997, 254(2):157-78; Dale et al., Meth. Mol. Biol., 1996, 57:369-74; Smith, Ann. Rev. Genet., 1985, 19:423-462; Botstein et al., Science, 1985, 229:1193-1201; Carter, Biochem. J., 1986, 237:1-7; Kramer et al., Cell, 1984, 38:879-887; Wells et al., Gene, 1985, 34:315-323; Minshull et al., Curr. Op. Chem. Biol., 1999, 3:284-290; Christians et al., Nat. Biotechnol., 1999, 17:259-264; Crameri et al., Nature, 1998, 391:288-291; Crameri, et al., Nat. Biotechnol., 1997, 15:436-438; Zhang et al., Proc. Nat. Acad. Sci. U.S.A., 1997, 94:4504-4509; Crameri et al., Nat. Biotechnol., 1996, 14:315-319; Stemmer, Nature, 1994, 366:389-391; Stemmer, Proc. Nat. Acad. Sci. USA, 1994, 91:10747-10751; EP 3 049 973; WO 95/22625; WO 97/0078; WO 97/35966; WO 98/27230; WO 00/42651; WO 01/75767; WO 2009/152336; and WO 2015/048573, all of which are incorporated herein by reference).

In some embodiments, the clones obtained following mutagenesis treatment are screened by subjecting the enzyme preparations to a defined treatment conditions or assay conditions (e.g., temperature, pH, type of ligase substrate (e.g., DNA, RNA, secondary structure, etc.), input substrate concentration, nucleotide cofactors, etc.) and measuring enzyme activity after the treatments or other suitable assay conditions. Clones containing a polynucleotide encoding the polypeptide of interest are then isolated from the gene, sequenced to identify the nucleotide sequence changes (if any), and used to express the enzyme in a host cell. Measuring enzyme activity from the expression libraries can be performed using any suitable method known in the art and as described in the Examples.

In some embodiments, for engineered polypeptides of known sequence, the polynucleotides encoding the enzyme can be prepared by standard solid-phase methods, according to known synthetic methods. In some embodiments, fragments of up to about 100 bases can be individually synthesized, then joined (e.g., by enzymatic or chemical ligation methods, or polymerase mediated methods) to form any desired continuous sequence (see, e.g., Hughes et al., Cold Spring Harb Perspect Biol. 2017 January; 9(1):a023812). For example, polynucleotides and oligonucleotides disclosed herein can be prepared by chemical synthesis using the classical phosphoramidite method (see, e.g., Beaucage et al., Tet. Lett., 1981, 22:1859-69; and Matthes et al., EMBO J., 1984, 3:801-05), as it is typically practiced in automated synthetic methods. According to the phosphoramidite method, oligonucleotides are synthesized (e.g., in an automatic DNA synthesizer), purified, annealed, ligated and cloned in appropriate vectors.

In some embodiments, a method for preparing the engineered RNA ligase polypeptide can comprise: (a) synthesizing a polynucleotide encoding a polypeptide comprising an amino acid sequence selected from the amino acid sequence of any variant as described herein, and (b) expressing the RNA ligase polypeptide encoded by the polynucleotide. In some embodiments of the method, the amino acid sequence encoded by the polynucleotide can optionally have one or several (e.g., up to 3, 4, 5, or up to 10) amino acid residue deletions, insertions and/or substitutions. In some embodiments, the amino acid sequence has optionally 1-2, 1-3, 1-4, 1-5, 1-6, 1-7, 1-8, 1-9, 1-10, 1-15, 1-20, 1-21, 1-22, 1-23, 1-24, 1-25, 1-30, 1-35, 1-40, 1-45, or 1-50 amino acid residue deletions, insertions and/or substitutions. In some embodiments, the amino acid sequence has optionally 1, 2, 3, 4, 5, 6, 7, 8, 9, 10, 11, 12, 13, 14, 15, 16, 17, 18, 19, 20, 21, 22, 23, 24, 25, 30, 30, 35, 40, 45, or 50 amino acid residue deletions, insertions and/or substitutions. In some embodiments, the amino acid sequence has optionally 1, 2, 3, 4, 5, 6, 7, 8, 9, 10, 11, 12, 13, 14, 15, 16, 18, 20, 21, 22, 23, 24, or 25 amino acid residue deletions, insertions and/or substitutions. In some embodiments, the substitutions are conservative or non-conservative substitutions.

In some embodiments, any of the engineered RNA ligase polypeptides expressed in a host cell are recovered and/or purified from the cells and/or the culture medium using any one or more of the known techniques for protein purification, including, among others, lysozyme treatment, sonication, filtration, salting-out, ultra-centrifugation, and chromatography, as described herein.

### Composition of RNA Ligases

In a further aspect, the present disclosure provides compositions of the RNA ligases disclosed herein. In some embodiments, the composition comprises at least one engineered RNA ligase polypeptide described herein. In some embodiments, the engineered RNA ligase polypeptide in the compositions is isolated or purified. In some embodiments, the RNA ligase is combined with other components and compounds to provide compositions and formulations comprising the engineered RNA ligase polypeptide as appropriate for different applications and uses.

In some embodiments, the composition comprises at least one engineered RNA ligase described herein. For example, a composition comprises at least one engineered RNA ligase provided in Tables 4.1, 5.1, 6.1, 7.1, 8.1, 9.1, 10.1, 11.1, and 12.1.

In some embodiments, the composition comprises an RNA ligase provided in Table 4.1. In some embodiments, the composition comprises an RNA ligase comprising an amino acid sequence comprising: 12 to 345 of SEQ ID NO: 2; residues 12 to 343 of SEQ ID NO: 4; residues 12 to 250 of SEQ ID NO: 6; residues 12 to 346 of SEQ ID NO: 8; residues 12 to 345 of SEQ ID NO: 10; residues 12 to 345 of SEQ ID NO: 12; residues 12 to 346 of SEQ ID NO: 14; residues 12 to 346 of SEQ ID NO: 16; residues 12 to 350 of SEQ ID NO: 18; residues 12 to 350 of SEQ ID NO: 20; or residues 12 to 344 of SEQ ID NO: 22. In some embodiments, the composition comprises an RNA ligase comprising an amino acid sequence comprising SEQ ID NO: 2, 4, 6, 8, 10, 12, 14, 16, 18, 20 or 22.

In some embodiments, the composition further comprises one or more of a buffer, a nucleotide substrate (e.g., ATP or dATP), and/or at least one or more polynucleotide substrates, for example a polynucleotide substrate with modified nucleotides.

In some embodiments, the composition further comprises an additive or ligation enhancing agent, including one or more of polyethylene glycol (e.g., PEG 6000, PEG 8000, etc.) or other molecular crowding reagents, including, among others, bovine serum albumin, Ficoll, and dextran (e.g., Dextran 6000).

In some embodiments, an engineered RNA ligase described herein is provided in solution, as a lyophilizate, or is immobilized on a substrate. In some embodiments, the substrate is a solid substrate, porous substrate, membrane, or particles. The enzyme can be entrapped in matrixes or membranes. In some embodiments, matrices include polymeric materials such as calcium-alginate, agar, k-carrageenin, polyacrylamide, agarose or derivatives thereof (e.g., cross-linked agarose), and collagen, or solid matrices, such as activated carbon, porous ceramic, and diatomaceous earth. In some embodiments, the matrix is a particle, a membrane, or a fiber. Types of membranes include, among others, nylon, cellulose, polysulfone, or polyacrylate.

In some embodiments, the enzyme is immobilized on the surface of a support material. In some embodiments, the enzyme is adsorbed on the support material. In some embodiments, the enzyme is immobilized on the support material by covalent attachment. Support materials include, among others, inorganic materials, such as alumina, silica, porous glass, ceramics, diatomaceous earth, clay, and bentonite, or organic materials, such as cellulose (CMC, DEAE-cellulose), starch, activated carbon, polyacrylamide, polystyrene, and ion-exchange resins, such as Amberlite, Sephadex, and Dowex.

Uses of Engineered RNA ligase Polypeptides and Kits

In another aspect, the present disclosure provides uses of the engineered RNA ligases for polynucleotide synthesis, RNA repair, or other molecular biological uses.

In some embodiments, the engineered RNA ligase is used for ligating polynucleotides and oligonucleotides. In some embodiments, the engineered RNA ligase is used for synthesizing polynucleotides from shorter oligonucleotides. In some embodiments, a method of ligating at least a first polynucleotide strand and a second polynucleotide strand comprises contacting a first polynucleotide strand and a second polynucleotide strand with an engineered RNA ligase described herein in presence of a nucleotide substrate under conditions suitable for ligation of the first polynucleotide strand to the second polynucleotide strand, wherein the first polynucleotide strand comprises a ligatable 5′-end and the second polynucleotide strand comprises a 3′-end ligatable to the 5′-end of the first polynucleotide strand.

In some embodiments, the second polynucleotide strand comprising a ligatable 3′-end has at least 2, 3, 4, 5 or 6 ribonucleotides at the 3′ terminal end. In some embodiments of the method, the 3′-end of the second polynucleotide strand is a 3′-OH. In some embodiments of the method, the 5′-end of the first polynucleotide strand is a 5′-phosphate. In some embodiments of the method, the internucleotide linkage contained in the first polynucleotide strand and/or second polynucleotide strand comprises a phosphate linkage.

In some embodiments, the method further comprises a third polynucleotide strand, wherein the first polynucleotide strand and second polynucleotide strand hybridize adjacent to one another on the third polynucleotide strand to position the 5′-end of the first polynucleotide strand adjacent to the 3′-end of the second polynucleotide strand, e.g., to form a nick. In some embodiments of the method, the third polynucleotide strand is continuous with the first polynucleotide strand or second polynucleotide strand.

In some embodiments of the method, the third polynucleotide strand is continuous with the first polynucleotide strand and second polynucleotide strand to form a single continuous polynucleotide ligase substrate. In some embodiments, the third polynucleotide strand is RNA, DNA, or a mixture of RNA and DNA.

In some embodiments of the method, the third polynucleotide comprises a splint or bridging polynucleotide, wherein the 5′-terminal sequence of the first polynucleotide strand and the 3′-terminal sequence of the second polynucleotide strand hybridize adjacent to one another on the splint or bridging polynucleotide to position the 5′-end of the first polynucleotide strand adjacent to the 3′-end of the second polynucleotide strand. In some embodiments, the splint or bridging polynucleotide is RNA, DNA, or a mixture of RNA and DNA.

In some embodiments, the first polynucleotide strand is hybridized to the third polynucleotide strand to form a first double stranded fragment, and the second polynucleotide strand is hybridized to a fourth polynucleotide strand to form a second double stranded fragment, where the first and second double stranded fragments can base pair to form a substrate for the engineered RNA ligase. In some embodiments, the first double stranded fragment and the second double stranded fragment have complementary overhangs or complementary single stranded ends, also referred to as cohesive or sticky ends, that can base pair and form double stranded nick(s) between the first and second double stranded fragments that serve as a substrate for the engineered RNA ligase.

In some embodiments, the complementary ends (e.g., sticky or cohesive ends) are of sufficient length for the double stranded fragments to base pair and form suitable substrates for the engineered RNA ligase. In some embodiments, the complementary single stranded end comprises at least 1-50, 2-40, 3-35, 4-30, 5-25, 6-20, or 8-15 or more nucleotides in length. In some embodiments, the complementary single stranded end is from 1-10, 2-8, or 4-6 nucleotides in length. In some embodiments, the complementary single stranded end is 1, 2, 3, 4, 5, 6, 8, 10, 12, 14, 16, 18, 20, or more nucleotides in length.

In some embodiments, the double stranded region or complementary portion of each of the double stranded fragment is from 4-50, 6-45, 8-40, 10-35, 12-30, or 14-25 nucleotides in length. In some embodiments, the double stranded region is at least 4, 5, 6, 7, 8, 9, 10, 12, 14, 16, 18, 20, 22, 24, 26, 28, 30, 35, 40, 45, or 50 nucleotides in length.

In some embodiments of the method, the ligase substrates comprise at least 2, 3, 4, 5, or 6 or more double stranded fragments, where each double stranded fragment has a complementary end that can base pair with at least one other double stranded fragment with a complementary end to form substrates for the RNA ligase. In some embodiments, the double stranded fragments have complementary or cohesive ends, e.g., at the 5′ and 3′ ends, such that the double stranded fragments can be ligated to form concatemers. In some embodiments, where the number of double stranded fragments is 3, at least one of the double stranded fragments has complementary ends at the 5′ and 3′ ends of the fragment, where double stranded fragment base pairs with two other different double stranded fragments to form a substrate that can be ligated to a product containing the three double stranded fragments. It is to be understood that where 3 or more double stranded fragments are ligated, the complementary ends of the double stranded fragments can be designed for ligation of all of the different double stranded fragments.

In some embodiments of the method, the first polynucleotide strand and/or second polynucleotide strand comprise one or more modified nucleotides or nucleotide analogs, wherein the modified nucleotide or nucleotide analog comprises a nucleobase analog or modified nucleobase, modified nucleoside sugar residue, modified internucleotide linkage, modified 5′-end phosphate group, and/or modified 3′-end hydroxyl group.

In some embodiments, the polynucleotide substrate comprises a modified 5′-end phosphate group, wherein the modified phosphate group is a phosphate analog. In some embodiments of the method, the first polynucleotide comprises a 5′-phosphate analog. In some embodiments, the 5′-phosphate analog is a phosphorothioate, phosphoramidate, monomethylphosphate, methylphosphonate, vinylphosphonate, or phosphonocarboxylate.

In some embodiments of the method, the polynucleotide substrate comprises one or more modified nucleoside sugar residues. In some embodiments of the method, the first polynucleotide and/or second polynucleotide comprises one or more modified nucleoside sugar residues. In some embodiments, the modified nucleoside sugar residue is a 2′-O-alkyl, a 2′-halo, a 3-D-ribo LNA, or a α-L-ribo-LNA (e.g., locked nucleic acids). In some embodiments, the modified nucleoside sugar residue is, among others, a 2′-O-methyl, 2′-O-ethyl, or 2′-O-propyl group. In some embodiments, the modified nucleoside sugar residue is 2′-fluoro, 2′-bromo, or 2′-chloro, preferably 2′-fluoro. In some embodiments, the sugar residue is modified with a conjugate, such as a targeting moiety, for example, GalNAc or lipid moieties.

In some embodiments of the method, the polynucleotide substrate comprises one or more modified nucleotide residues having a modified nucleobase or nucleobase analog. In some embodiments of the method, the first polynucleotide and/or second polynucleotide comprises one or more modified nucleotide residues having a modified nucleobase or nucleobase analog. In some embodiments, the nucleobase analog is, among others, xanthine, hypoxanthine, inosine, 6-methyladenine, 7-methylguanine, 2,6-diaminopurine, 5-methylcytosine, 5-hydroxycytosine, 5-bromocytosine, 5-iodocytosine, 2-thiothymine, 5-fluorouracil, 5-bromouracil, 8-bromoguanine, 8-aminoguanine, or 8-aza-7-deazaguanine. In some embodiments, the nucleobase is modified with a conjugate, such as a targeting moiety, for example, GalNAc and lipid moieties.

In some embodiments of the method, the polynucleotide substrate comprises one or more modified or non-standard internucleotide linkages, i.e., internucleotide linkages other than phosphate linkages. In some embodiments, the first polynucleotide and/or second polynucleotide comprises one or more modified or non-standard internucleotide linkages. In some embodiments, the internucleotide linkage is a phosphorothioate, phosphoacetate, phosphoramidate, methylphosphonate, or phosphonocarboxylate. In some embodiments, at least 1%, 2%, 5%, 10%, or more of the internucleotide linkages are non-standard internucleotide linkages. In some embodiments, the non-standard internucleotide linkages are present 1, 2, 3, 4 or 5 nucleotides of the 5′ and/or 3′-terminus of the polynucleotide substrates.

In some embodiments of the method, the polynucleotide substrate comprises modified 3′-hydroxyl group. In some embodiments, 3′-end of a polynucleotide substrate is attached to a matrix or surface, such as a solid matrix. In such instances, the polynucleotide substrate attached to a matrix has a ligatable 5′-end. In some embodiments, the 3′-end of a polynucleotide substrate is modified with an amine, halo, phosphate, phosphate analog, —O-alkyl, lipid moiety, or a detectable label.

In some embodiments, the polynucleotide substrate comprises C-4′ modifications, including among others, 4′-thio-C2′ modifications, 4′/5′ aminoalkyl/C2′ modifications, C4′-Guanidino-C2′-modifications, and C4′-O-Me/C2′-modifications (see, e.g., Gangopadhyay et al., RNA Biology, 2022, 19:1, 452-467.

In some embodiments, the method includes a nucleotide substrate used by the RNA ligase to catalyze the joining reaction. In some embodiments, the nucleotide substrate is ATP and/or dATP.

Preferably, the nucleotide substrate is ATP. In some embodiments, the reaction conditions for the ligation include additional components, such as Mg⁺², buffer, and/or salts.

In some embodiments, the reaction conditions also include a ligation enhancing reagent, including, among others, polyethylene glycol (e.g., PEG 6000 and PEG 8000) or other molecular crowding reagents, for example bovine serum albumin (BSA), dextran, and Ficoll.

In some embodiments, ligation reaction is carried out at a suitable temperature and reaction time period. In some embodiments, the ligation reaction temperature is from about 2° C. to about 60° C. In some embodiments, the ligation reaction temperature is from 4° C. to 55° C., 4° C. to 50° C., 4° C. to 45° C., or 10° C. to 40° C. In some embodiments, the ligation reaction temperature is 2° C., 5° C., 10° C., 15° C., 20° C., 25° C., 30° C., 37° C., 40° C., 45° C., 50° C., 55° C., or 60° C. In some embodiments, the ligation reaction temperature can use different temperatures, for example a temperature at which stable hybrids are formed between polynucleotide substrate(s) followed by a higher temperature to promote completion of ligation reaction.

In some embodiments, the ligation reaction time can be a sufficient time for ligation of polynucleotide substrate(s). In some embodiments, the ligation reaction time is from 0.5-72 hr or longer.

In some embodiments, the ligation reaction time is 1-72 hr, 2-48 hr, or 2-24 hr. In some embodiments, the ligation reaction time is 0.5, 1, 2, 4, 5, 12, 24, 48, or 72 hr or longer.

In some embodiments, the engineered RNA ligase is used to ligate nicks or related nick structures (see, e.g., Cheng et al., Royal Soc Chem Adv., 2019, 9:8620-8627). In some embodiments, the engineered RNA ligase is used to synthesize RNAs from shorter oligonucleotides, for example by use of splint nucleic acids (see, e.g., Stark et al., RNA, 2006, 12:2014-2019). In some embodiments, the engineered RNA ligase is used to ligate modified oligonucleotides, such as provided in the Examples. Other examples of modified oligonucleotide products that can be synthesized using short oligonucleotide substrates, include, among others, shRNAs or siRNAs described in patent publications WO22104366, WO22029209, WO22031847, WO20226960, US2022072024, US2021238606, U.S. Ser. No. 11/286,488, US2017305956, WO22212153, WO22192519, WO22125490, WO22072447, WO21257568, WO21102373, WO21072395, WO21022108, US2022079971, U.S. Ser. No. 11/034,957, US2021332365, U.S. Ser. No. 11/015,201, U.S. Ser. No. 10/995,336, U.S. Ser. No. 11/091,759, U.S. Ser. No. 10/889,813, U.S. Ser. No. 10/130,651, U.S. Ser. No. 10/513,703, WO19232255, WO21108640, WO22147304, and WO21138537.

In a further aspect, the present disclosure provides a kit comprising an engineered RNA ligase described herein. In some embodiments, the kit further comprises one or more of a buffer, a nucleotide substrate (e.g., ATP or dATP), and/or one or more polynucleotide substrates. In some embodiments, the kit further comprises an additive or ligation enhancing agent, including one or more of polyethylene glycol (e.g., PEG 6000, PEG 8000, etc.) or other molecular crowding reagents, including, among others, bovine serum albumin, Ficoll, and dextran (e.g., Dextran 6000).

### EXAMPLES

The following Examples, including experiments and results achieved, are provided for illustrative purposes only and are not to be construed as limiting the present invention.

In the experimental disclosure below, the following abbreviations where relevant apply: ppm (parts per million); M (molar); mM (millimolar), uM and μM (micromolar); nM (nanomolar); mol (moles); gm and g (gram); mg (milligrams); ug and μg (micrograms); L and 1 (liter); ml and mL (milliliter); ul, uL, μl, and μL (microliter); cm (centimeters); mm (millimeters); um and μm (micrometers); sec. (seconds); min(s) (minute(s)); h(s) and hr(s) (hour(s)); U (units); OD (optical density); MW (molecular weight); rpm (rotations per minute); rcf (relative centrifugal force); psi and PSI (pounds per square inch); ° C. (degrees Celsius); RT and rt (room temperature); NGS (next-generation sequencing); ds (double stranded); ss (single stranded); CDS (coding sequence); DNA (deoxyribonucleic acid); RNA (ribonucleic acid); E. coli W3110 (commonly used laboratory E. coli strain, available from the Coli Genetic Stock Center [CGSC], New Haven, CT); HTP (high throughput); HPLC (high pressure liquid chromatography); FPLC (fast protein liquid chromatography); ddH2O (double distilled water); PBS (phosphate buffered saline); BSA (bovine serum albumin); DTT (dithiothreitol); CAM (chloramphenicol); CAT (chloramphenicol acetyltransferase); IPTG (isopropyl β-D-1-thiogalactopyranoside); FIOPC (fold improvements over positive control); FIOP (fold improvements over parent); LB (Luria-Bertani); TB (Terrific-Broth).

### Example 1

E. coli Expression Hosts Containing Recombinant RNA Ligase 2 (Rnl2) Genes

To produce enzyme, RNA ligase 2 genes were cloned in either of two different expression vectors. For Examples 3-6, pCK110900 (see, e.g., FIG. 3 of US Pat. Appln. Publn. No. 2006/0195947) operably linked to the lac promoter under control of the lac1 repressor was used. The expression vector also contains the P15a origin of replication and the chloramphenicol resistance gene. Example 7-13 used the pJV vector system (see U.S. patent Ser. No. 10/184,117). The resulting plasmids were transformed into E. coli W3110, using standard methods known in the art. The transformants were isolated by subjecting the cells to chloramphenicol selection, as known in the art (see e.g., U.S. Pat. No. 8,383,346 and WO2010/144103).

### Example 2

### Preparation of RNA Ligase (Rnl2)-Containing Wet Cell Pellets

E. coli. cells containing recombinant Rnl2-encoding genes from monoclonal colonies were inoculated into 180 μl LB containing 1% glucose and 30 μg/mL chloramphenicol (CAM) in the wells of 96-well, shallow-well microtiter plates. The plates were sealed with O₂-permeable seals, and cultures were grown overnight at 30° C., 200 rpm, and 85% humidity. Then, 10 μl of each of the cell cultures were transferred into the wells of 96-well, deep-well plates containing 390 mL TB and 30 μg/mL CAM. The deep-well plates were sealed with O₂-permeable seals and incubated at 30° C., 250 rpm, and 85% humidity until OD600 0.6-0.8 was reached. The cell cultures were then induced by IPTG to a final concentration of 1 mM and incubated overnight under the same conditions as originally used. The cells were then pelleted using centrifugation at 4,000 rpm for 10 min. The supernatants were discarded, and the pellets were frozen at −80° C. prior to lysis.

### Example 3

E. coli Shake Flake Expression and Purification of Recombinant RNA Ligase 2 (Rnl2) Genes

To determine the first round backbone, a set of ten ligase enzymes were selected based on their homology to SEQ ID NO: 2. HTP cultures grown as described above were plated onto LB agar plates with 1% glucose and 30 μg/ml chloramphenicol and grown overnight at 37° C. A single colony from each culture was transferred to 6 ml of LB broth with 1% glucose and 30 μg/ml chloramphenicol. The cultures were grown for 18 h at 30° C., 250 rpm, and subcultured at a dilution of approximately 1:50 into 250 ml of Terrific Broth with 30 μg/ml of chloramphenicol, to a final OD600 of approximately 0.05. The cultures were incubated for approximately 3 h at 30° C., 250 rpm, to an OD600 of 0.6-0.8, and then induced with the addition of IPTG at a final concentration of 1 mM. The induced cultures were incubated for 20 h at 30° C., 250 rpm. Following this incubation period, the cultures were centrifuged at 4000 rpm×10 min. The culture supernatant was discarded, and the pellets were resuspended in 35 ml of 50 mM Tris-HCl, pH 7.5. This cell suspension was chilled in an ice bath and lysed using a Microfluidizer cell disruptor (Microfluidics M-110L). The crude lysate was pelleted by centrifugation (16,000 rpm for 60 min at 4° C.), and the supernatant was then filtered through a 0.2 μm PES membrane to further clarify the lysate. These RNA ligase 2 lysates were supplemented with 20 mM imidazole and 500 mM NaCl. Lysates were then purified using an ÄKTA Pure purification system and a 5 ml HisTrap FF column (GE Healthcare) using the run parameters provided below in Table 1. The wash buffer comprised 50 mM Tris-HCl pH 8, 500 mM NaCl, 20 mM imidazole, and the elution buffer comprised 50 mM Tris-HCl, pH 8, 500 mM NaCl, 300 mM imidazole:

TABLE 1
FPLC Run Parameters

| Parameter | Volume | Units |
|---|---|---|
| Column volume | 5 | ml |
| Flow rate | 12 | ml/min |
| Pressure limit pre-column | 0.5 | MPa |
| Pressure limit delta-column | 0.3 | MPa |
| Sample volume | 50 | mls |
| Equilibration volume | 5 | CV |
| Wash Unbound volume | 15 | CV |
| Isocratic (step) Elution volume | 5 | CV |
| Fraction volume | 1.5 | mls |
| RE-equilibration volume | 5 | CV |

The most concentrated fractions were identified by UV absorption (A280) and pooled; 3 ml of eluate was dialyzed overnight in 1×ligase storage buffer (10 mM Tris-HCl pH 7.5, 50 mM KCl, 35 mM ammonium sulfate, 50% glycerol) overnight in a 3K Slide-A-Lyzer™ dialysis cassette (Thermo Fisher) for buffer exchange. RNA ligase 2 concentrations in the preparations were measured by absorption at 280 nm.

### Example 4

Improvements Over SEQ ID NO: 2 in qPCR Amplification Activity

The oligonucleotides in Table 2 were used to assay RNA ligase activity. Each of the oligonucleotides are in the 5′-3′ direction.

TABLE 2
RNA Ligase Oligonucleotide Substrates

| Ligase Substrate Name | Sequence |
|---|---|
| sense strand fragment 1 | UUGCCAAGCUUGGU (SEQ ID NO: 583) |
| sense strand fragment 2 | CAUCUAGCAG (SEQ ID NO: 584) |
| sense strand fragment 3 | CCGAAAGGCUGC (SEQ ID NO: 585) |
| sense strand fragment 4 | CAUCUAGCAGCCGAAAGG (SEQ ID NO: 586) |
| sense strand fragment 5 | CUGC |
| antisense strand fragment 1 | AGCUUGGCAAGG (SEQ ID NO: 587) |
| antisense strand fragment 2 | UAGAUGACCA (SEQ ID NO: 588) |
| antisense strand fragment 3 | GCAAGG |
| antisense strand fragment 4 | AGCUUG |

The oligonucleotides in Table 2 have the following features. Sense strand fragment 1 has a 2-O-methyl on nucleotide residues 1-7 and 12-14, a 2′-fluoro on nucleotide residues 8-11, a 5′-OH terminus, a 3′-OH terminus, and a phosphorothioate internucleotide linkage between nucleotide residues 1-2. Sense strand fragment 2 has a 2′-O-methyl on nucleotide residues 1-10, a 5′-phosphate terminus, and a 3-OH terminus. Sense fragment 3 has 2′-O-methyl on nucleotide residues 1-3 and 7-12, a 5′-phosphate terminus, a 3′-OH terminus, and a GalNac moiety attached to the 2′ position of each nucleotide residues 4-6, as shown below. Sense fragment 4 has 2′-O-methyl on nucleotide residues 1-13 and 17-18, a 5′-phosphate terminus, a 3′-OH terminus, and a GalNac attached to the 2′ position of each of nucleotide residues 14-16, as shown below. Sense strand fragment 5 has 2′-O-methyl on nucleotide residues 1-4, a 5′-phosphate terminus, and a 3′-OH terminus. Antisense fragment 1 has 2′-O-methyl on nucleotide residues 1-3 and 5-12, a 2′-fluoro on nucleotide residue 4, a 5′-phosphate terminus, a 3′-OH terminus, and a phosphorothioate internucleotide linkage between nucleotide residues 10-11 and 11-12. Antisense fragment 2 has 2′-O-methyl on nucleotide residues 1, 6, 8, and 9, a 2′-fluoro on nucleotide residues 2-5, 7, and 10, a 5′-monomethylphosphate terminus, a 3′-OH terminus, and a phosphorothioate internucleotide linkage between each of residues 1-2, 2-3, and 3-4. Antisense fragment 3 has 2′-O-methyl on nucleotide residues 1-6, a 5′-phosphate terminus, a 3′-OH terminus, and a phosphorothioate internucleotide linkage between each of nucleotide residues 4-5 and 5-6. Antisense fragment 4 has 2′-O-methyl on nucleotide residues 1-3 and 5-6, a 2′-fluoro on nucleotide residue 4, a 5′-phosphate terminus, and a 3′-OH terminus. The combination of oligonucleotides used in the RNA ligase reactions are provided in Table 3.

TABLE 3

| Reaction # | Oligonucleotides |
|---|---|
| 1 | sense strand fragment 1 |
| | sense strand fragment 4 |
| | sense strand fragment 5 |
| | antisense strand fragment 1 |
| | antisense strand fragment 2 |
| 2 | sense strand fragment 1 |
| | sense strand fragment 2 |
| | sense strand fragment 3 |
| | antisense strand fragment 1 |
| | antisense strand fragment 2 |
| 3 | sense strand fragment 1 |
| | sense strand fragment 4 |
| | sense strand fragment 5 |
| | antisense strand fragment 2 |
| | antisense strand fragment 3 |
| | antisense strand fragment 4 |
| 4 | sense strand fragment 1 |
| | sense strand fragment 2 |
| | sense strand fragment 3 |
| | antisense strand fragment 2 |
| | antisense strand fragment 3 |
| | antisense strand fragment 4 |

The GalNac residues are attached to the 2′ position of the adenine nucleotide residues as follows:

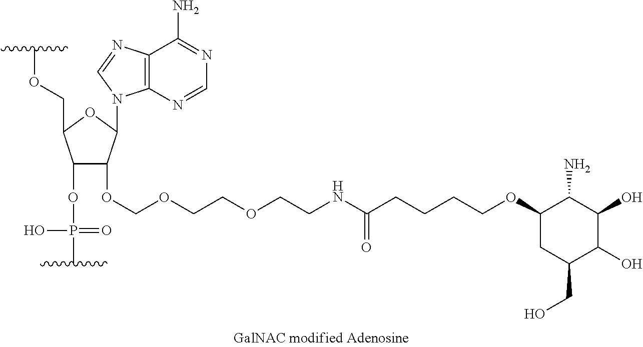 [SMILES: *OCC1OC(n2cnc3c(N)ncnc32)C(OCOCCOCCNC(=O)CCCCO[C@@H]2C[C@H](CO)C(O)[C@H](O)[C@H]2N)C1OP(*)(=O)O]

The target product of each of the ligation reactions in Table 3 has the following sequences: Product 1—UUGCCAAGCUUGGUCAUCUAGCAGCCGAAAGGCUGC (SEQ NO: 589) and Product 2—UAGAUGACCAAGCUUGGCAAGG (SEQ NO: 590), where each of the products have the modifications in the substrate oligonucleotides, and where Product 1 and Product 2 are hybridized to each other to form a shRNA with the GalNac ligands.

To test sample activity, 0.1 g/L protein was assayed in reactions containing 50 mM Tris-HCl buffer at pH 7.5, 2 mM MgCl₂, 1 mM DTT, 0.4 mM ATP, and 0.4 mM of an equimolar mixture of the fragments compromising either of the 4 reactions (Reaction 1: sense strands 1, 4, and 5, and antisense strands 1-2; Reaction 2: sense strands 1-3, and antisense strands 1-2; Reaction 3: sense strands 1, 4 and 5, and antisense strands 2-4; or Reaction 4: sense strands 1-3, and antisense strands 2-4. The reactions were mixed before being incubated for 2 hours at 37° C. in a PCR thermocycler. Afterwards, EDTA was added to each reaction to a final concentration of 10.0 mM and the samples analyzed via UPLC to quantify the remaining fragment concentrations and product yields.

The % Conversions relative to SEQ ID NO: 2 were calculated as in the following:

$$\% conversion = \frac{area under the curve for all products}{\begin{matrix}Total area under the curve for all \\ products and remaining fragments\end{matrix}} * 100$$

using Analytical method 13.1, and the results are shown in Table 4.1.

TABLE 4.1
Relative to SEQ ID NO: 2

| SEQ ID | Reaction 1 % Conv. | Reaction 2 % | Reaction 3 % Conv. | Reaction 4 % Conv. |
|---|---|---|---|---|
| NO: | Relative to SEQ ID | Conv. Relative to | Relative to SEQ ID | Relative to SEQ ID |
| (nt/aa) | NO: 2 | SEQ ID NO: 2 | NO: 2 | NO: 2 |
| 1/2 | +++ | +++ | + | + |
| 3/4 | ++ | ++ | + | + |
| 5/6 | + | + | + | + |
| 7/8 | + | + | + | + |
| 9/10 | + | + | + | + |
| 11/12 | ++ | ++ | +++ | ++ |
| 13/14 | +++ | +++ | +++ | +++ |
| 15/16 | + | + | ++ | + |
| 17/18 | + | + | ++ | +++ |
| 19/20 | ++ | + | + | ++ |
| 21/22 | + | ++ | ++ | ++ |
| | Levels of increased | Levels of increased | Levels of increased | Levels of increased |
| | activity were | activity were | activity were | activity were |
| | determined relative | determined relative | determined | determined relative to |
| | to the reference | to the reference | relative to the | the reference |
| | polypeptide of SEQ | polypeptide of SEQ | reference polypeptide | polypeptide of SEQ |
| | ID NO: 2 and | ID NO: 2 and | of SEQ ID | ID NO: 2 and defined |
| | defined as follows: | defined as follows: | NO: 2 and defined as | as follows: “+” 5.21 to |
| | “+” .37 to 72.44 (first | “+” .00 to 57.00 | follows: “+” 5.59 to | 53.26 |
| | 50%), “++” > 72.44 | (first 50%), “++” > | 37.02 (first 50%), | (first 50%), “++” > |
| | (next 30%), “+++” > | 57.00 (next 30%), | “++” > 37.02 (next | 53.26 (next 30%), |
| | 89.03 (top 20%) | “+++” > 77.50 (top | 30%), “+++” > 40.68 | “+++” > 54.51 (top |
| | | 20%) | (top 20%) | 20%) |

To test thermostability, each sample was incubated at 1.5 g/L in 40 mM Tris-HCl at pH 7.5, 100 mM KCl, 0.1 mM EDTA for 1 hour at room temperature and from 30 to 80° C. After incubation, the activity of each sample was tested: 0.05 g/L protein was assayed in reactions containing 50 mM Tris-HCl buffer at pH 7.5, 2 mM MgCl₂, 1 mM DTT, 0.4 mM ATP, and 0.4 mM of an equimolar mixture of the fragments compromising Reaction 2 (sense strands 1-3, and antisense strands 1-2). The reactions were mixed before being incubated for 2 hours at 37° C. in a PCR thermocycler. Afterwards, EDTA was added to each reaction to a final concentration of 10.0 mM and the samples analyzed via UPLC to quantify the remaining fragment concentrations and product yields.

Using plots of % residual activity vs. incubation temperature, the temperature at which 50% of the untested sample relative to SEQ ID NO: 2 were calculated as in the following:

$$\% residual activity = \frac{\% conversion at temperature}{\% conversion sample at room temperature} * 100$$

using Analytical method 13.1, and the results are shown in Table 4.2.

TABLE 4.2
Relative to SEQ ID NO: 2

| SEQ ID NO: (nt/aa) | Stability Ranking Relative to SEQ ID NO: 2 |
|---|---|
| 1/2 | + |
| 3/4 | + |
| 5/6 | +++ |
| 7/8 | +++ |
| 9/10 | + |
| 11/12 | + |
| 13/14 | + |
| 15/16 | ++ |
| 17/18 | ++ |
| 19/20 | + |
| 21/22 | ++ |

Levels of increased activity were determined relative to the reference polypeptide of SEQ ID NO: 2 and defined as follows: “+” 1.00 to 1.00 (first 50%), “++” > 1.00 (next 30%), “+++” > 3.00 (top 20%)

### Example 5

### Improvements Over SEQ ID NO: 14 in RNA Ligase 2 Activity

SEQ ID NO: 14 was selected as the parent enzyme after screening for activity on all four reaction ligation scenarios and for stability. Libraries of engineered genes were produced using established techniques (e.g., saturation mutagenesis, recombination of previously identified beneficial mutations). The polypeptides encoded by each gene were produced in HTP as described in Example 1, and the cell paste was generated as described in Example 2.

To prepare cells for lysis, 300 μl of 0.1 mg/mL lysozyme in 50 mM Tris-HCl buffer at pH 7.5 was added to the cell paste of each sample. After thorough resuspension, the cells were incubated at 40° C. for 1 hour in a PCR thermocycler, then again at 44° C. for another hour. The samples were then centrifuged for 10 minutes at 4000 rpm and 4° C. and the clear supernatants used in subsequent biocatalytic reactions.

To screen, 20%(v/v) lysate was transferred into reactions containing 50 mM Tris-HCl buffer at pH 7.5, 2 mM MgCl2, 1 mM DTT, 0.4 mM ATP, and 0.4 mM of an equimolar mixture of the fragments compromising Reaction 2 (sense strands 1-3, and antisense strands 1-2). The reactions were mixed before being incubated for 16 hours at 37° C. in a PCR thermocycler. Afterwards, EDTA was added to each reaction to a final concentration of 18.3 mM and the samples analyzed via UPLC to quantify the remaining fragment concentrations and product yields.

Using peak area from Analytical method 13.1, the % conversion for each sample was calculated as shown here:

$$\% conversion = \frac{area under the curve for all products}{\begin{matrix}Total area under the curve for all \\ products and remaining fragments\end{matrix}} * 100$$

Activity relative to SEQ ID NO: 14 (FIOP) was calculated as the % conversion formed by the variant over % conversion of SEQ ID NO: 14 and shown in Table 5.1.

TABLE 5.1
Relative to SEQ ID NO: 14

| SEQ | | FIOP |
|---|---|---|
| ID NO: | Amino Acid Differences | to SEQ ID |
| (nt/aa) | (Relative to SEQ ID NO: 14) | NO: 14 |
| 23/24 | V177P/T260S/N307E/L345V | +++ |
| 25/26 | A171E/Q175K/I183V/S280N/L283I/K303Q | +++ |
| 27/28 | M95V/L159F/V177P/T260S/S288E | +++ |
| 29/30 | Q175K/I183V/A247K/S280N/L283I/R296K | +++ |
| 31/32 | V177P/N307E | +++ |
| 33/34 | H81Y/A171E/I1183V/N335D | +++ |
| 35/36 | Q175K/I183V | +++ |
| 37/38 | H81Y/Q175K/I183V/S280N/R296K/N335D/S339P | +++ |
| 39/40 | H81Y/I183V/A247K/V266L | +++ |
| 41/42 | M95V/V177P | +++ |
| 43/44 | A171E/S280N/L283I/R296K/K303Q | ++ |
| 45/46 | M85L/V177P/T260S/S288E/I333V/L345V | ++ |
| 47/48 | V177P/A291P/N307E/I333V | ++ |
| 49/50 | H81Y/A171E/Q175K/I183V/M316L/I337L | ++ |
| 51/52 | H81Y/A171E/V266L/S280N/L283I/N335D/S339P | ++ |
| 53/54 | V177P | ++ |
| 55/56 | M95V/L159F/V177P/T260S/S280N/S288E/I306L/L345V | ++ |
| 57/58 | A171E/Q175K/N335D | ++ |
| 59/60 | V177P/T260S/I306L/I333V | ++ |
| 61/62 | A14K/M95V/V177P/S288E/N307E | ++ |
| 63/64 | M95V/V177P/L345V | ++ |
| 65/66 | M95V/L159F/V177P/T260S/H285K/S288E | ++ |
| 67/68 | V266L/R296K | ++ |
| 69/70 | H81Y/L283I/N335D/I337L | ++ |
| 71/72 | M95V/L159F/V177P/T260S/H342I/L345V | ++ |
| 73/74 | I63V/A171E/Q175K/I183V/V266L/S280N/R296K/M316L | ++ |
| 75/76 | S280N/N335D/I337L | + |
| 77/78 | H81Y/A171E/S280N | + |
| 79/80 | H81Y/A171E/Q175K/R296K/M316L | + |
| 81/82 | A171E/Q175K/M316L/N335D | + |
| 83/84 | A171E/S280N/L283I/R296K/M316L | + |
| 85/86 | A171E/L283I/N335D | + |
| 87/88 | I63V/A171E/Q175K/I183V/V266L/L283I/K303Q/M316L | + |
| 89/90 | A171E/A247K/V266L/L283I/R296K/K303Q/M316L/ | + |
| | N335D | |
| 91/92 | I63V/H81Y/A171E/Q175K/I183V/V266L/S280N/L283I/ | + |
| | I337L | |
| 93/94 | H81Y/A171E/K303Q/M316L/N335D | + |
| 95/96 | S288E/N307E | + |
| 97/98 | A14K/M95V/L159F/V177P/L345V | + |
| 99/100 | H81Y/A171E/Q175K/I183V/A247K/V266L/S280N/L283I | + |
| 101/102 | H81Y/A247K/V266L | + |
| 103/104 | S280N | + |
| 105/106 | M85L/V177P/T260S | + |
| 107/108 | I63V/I183V | + |
| 109/110 | Q175K/I183V/A247K/S280N/L283I/M316L | + |
| 111/112 | A291P | + |
| 113/114 | A247K | + |
| 115/116 | A171E/S280N/L283I | + |
| 117/118 | H81Y/A171E/Q175K/S280N/M316L | + |
| 119/120 | I333V | + |
| 121/122 | A171E/R296K/M316L | + |
| 123/124 | H81Y/A171E | + |
| 125/126 | S280N/M316L/N335D | + |

Levels of increased activity were determined relative to the reference polypeptide of SEQ ID NO: 14 and defined as follows: “+” 1.81 to 3.45 (first 50%), “++” > 3.45 (next 30%), “+++” > 6.07 (top 20%).

### Example 6

### Improvements Over SEQ ID NO: 42 in RNA Ligase 2 Activity

SEQ ID NO: 42 was selected as the parent enzyme after screening variants as described in Example 5 above. Libraries of engineered genes were produced using established techniques (e.g., saturation mutagenesis, recombination of previously identified beneficial mutations). The polypeptides encoded by each gene were produced in HTP as described in Example 1 and the cell paste was generated as described in Example 2.

To prepare cells for lysis, 300 μl of 0.1 mg/mL lysozyme in 50 mM Tris-HCl buffer at pH 7.5 was added to the cell paste of each sample. After thorough resuspension, the cells were incubated at 45° C. for 1 hr in a PCR thermocycler. The samples were then centrifuged for 10 minutes at 4000 rpm and 4° C. and the clear supernatants used in subsequent biocatalytic reactions.

To screen, 20%(v/v) lysate was transferred into reactions containing 50 mM Tris-HCl buffer at pH 7.5, 2 mM MgCl₂, 1 mM DTT, 0.4 mM ATP, and 0.4 mM of an equimolar mixture of the fragments compromising Reaction 2 (sense strands 1-3, and antisense strands 1-2). The reactions were mixed before being incubated for 16 hours at 37° C. in a PCR thermocycler. Afterwards, EDTA was added to each reaction to a final concentration of 18.3 mM and the samples analyzed via UPLC to quantify the remaining fragment concentrations and product yields.

Using peak area from Analytical method 13.1, the % conversion for each sample was calculated as shown here:

$$\% conversion = \frac{area under the curve for all products}{\begin{matrix}Total area under the curve for all \\ products and remaining fragments\end{matrix}} * 100$$

Activity relative to SEQ ID NO: 42 (FIOP) was calculated as the % conversion formed by the variant over % conversion of SEQ ID NO: 42 and shown in Table 6.1.

TABLE 6.1
Relative to SEQ ID NO: 42

| SEQ | Amino Acid | Amino Acid | FIOP |
|---|---|---|---|
| ID | Differences | Differences | Relative to |
| NO: | (Relative to | (Relative to | SEQ ID |
| (nt/aa) | SEQ ID NO: 14) | SEQ ID NO: 42) | NO: 42 |
| 223/224 | M95V/V149R/G156T/V177P/D195G | V149R/G156T/D195G | +++ |
| 225/226 | K15M/M95V/V149T/V177P/Y186R/ | K15M/V149T/Y186R/N224Y/ | +++ |
| | N224Y/L317A | L317A | |
| 227/228 | K15P/M95V/G156T/V177P/D195G/ | K15P/G156T/D195G/N224Y/ | +++ |
| | N224Y/K226I/L317A/I331L | K226I/L317A/I331L | |
| 229/230 | K15M/M95V/V177P/D195G/L317A/ | K15M/D195G/L317A/I331R | +++ |
| | I331R | | |
| 231/232 | M95V/V149R/V177P/N224Y/I331R | V149R/N224Y/I331R | +++ |
| 233/234 | K15E/M95V/V177P/D195G/N224Y/ | K15E/D195G/N224Y/K226I/ | +++ |
| | K226I/L317A/I331W | L317A/I331W | |
| 235/236 | K15P/G33L/K86G/M95V/V149R/ | K15P/G33L/K86G/V149R/ | +++ |
| | A154C/V177P/D195G/L317A | A154C/D195G/L317A | |
| 237/238 | K15M/K86G/M95V/A154C/G156T/ | K15M/K86G/A154C/G156T/ | +++ |
| | V177P/Y186R/D195G/N224Y/K226I/ | Y186R/D195G/N224Y/K226I/ | |
| | I331L | I331L | |
| 239/240 | K15P/M95V/V149R/V177P/N224Y/ | K15P/V149R/N224Y/L317A/ | +++ |
| | L317A/I331R | I331R | |
| 241/242 | K15M/M95V/A154C/G156T/V177P/ | K15M/A154C/G156T/Y186R/ | +++ |
| | Y186R/L317A/I331R | L317A/I331R | |
| 243/244 | K15M/G33L/M95V/V149R/V177P/ | K15M/G33L/V149R/N224Y/ | +++ |
| | N224Y/K226I/I331W | K226I/I331W | |
| 245/246 | K15E/M95V/V177P/Y186R/N224Y/ | K15E/Y186R/N224Y/K226I/ | +++ |
| | K226I/L317A/I331R | L317A/I331R | |
| 247/248 | K15Y/G33L/M95V/V149R/V177P/ | K15Y/G33L/V149R/Y186R/ | +++ |
| | Y186R/N224Y/I331R | N224Y/I331R | |
| 249/250 | M95V/V177P/D195G/N224Y/I331R | D195G/N224Y/I331R | +++ |
| 251/252 | K15P/K86G/M95V/G156T/V177P/ | K15P/K86G/G156T/Y186R/D195 | +++ |
| | Y186R/D195G/K226I/L317A/I331L | G/K226I/L317A/I331L | |
| 253/254 | K15M/M95V/V177P/D195G/K226I/ | K15M/D195G/K226I/I331W | +++ |
| | I331W | | |
| 255/256 | M95V/G156T/V177P/Y186R/N224Y/ | G156T/Y186R/N224Y/K226I/ | +++ |
| | K226I/I331R | I331R | |
| 257/258 | K15Y/M95V/V149R/A154C/G156T/ | K15Y/V149R/A154C/G156T/ | +++ |
| | V177P/N224Y/K226I | N224Y/K226I | |
| 259/260 | G33L/M95V/G156T/V177P/Y186R/ | G33L/G156T/Y186R/D195G/ | +++ |
| | D195G/I331L | I331L | |
| 261/262 | K15P/K86G/M95V/V149R/A154C/ | K15P/K86G/V149R/A154C/ | +++ |
| | G156T/V177P/L317A/I331R | G156T/L317A/I331R | |
| 263/264 | K15M/M95V/V177P/Y186D/L317A/ | K15M/Y186D/L317A/I331R | +++ |
| | I331R | | |
| 265/266 | K15P/K86G/M95V/V149R/A154C/ | K15P/K86G/V149R/A154C/ | +++ |
| | V177P/D195G/I331L | D195G/I331L | |
| 267/268 | K15Y/G33L/M95V/V149T/A154C/ | K15Y/G33L/V149T/A154C/ | +++ |
| | G156T/V177P/K226I | G156T/K226I | |
| 269/270 | K15P/M95V/V149R/A154C/V177P/ | K15P/V149R/A154C/Y186R/ | +++ |
| | Y186R/L317A/I331L | L317A/I331L | |
| 271/272 | M95V/V177P/D195G/I331R | D195G/I331R | +++ |
| 273/274 | K15M/K86G/M95V/G156T/V177P/ | K15M/K86G/G156T/Y186R/ | +++ |
| | Y186R/D195G/I331W | D195G/I331W | |
| 275/276 | M95V/V149R/G156T/V177P/N224Y/ | V149R/G156T/N224Y/K226I/ | ++ |
| | K226I/I331W | I331W | |
| 277/278 | K15P/M95V/V149R/A154C/G156T/ | K15P/V149R/A154C/G156T/ | ++ |
| | V177P/Y186R/L317A/I331L | Y186R/L317A/I331L | |
| 279/280 | K15Y/M95V/A154C/V177P/Y186D/ | K15Y/A154C/Y186D/D195G | ++ |
| | D195G/ | | |
| 281/282 | K15E/K86G/M95V/V177P/D195G/ | K15E/K86G/D195G/N224Y/ | ++ |
| | N224Y/K226I | K226I | |
| 283/284 | M95V/V177P/N230D | N230D | ++ |
| 285/286 | K15P/G33L/M95V/G156T/V177P/ | K15P/G33L/G156T/I331R | ++ |
| | I331R | | |
| 287/288 | K15L/G33L/M95V/G156T/V177P/ | K15L/G33L/G156T/D195G/ | ++ |
| | D195G/N224Y/K226I | N224Y/K226I | |
| 289/290 | G33L/M95V/V149R/G156T/V177P/ | G33L/V149R/G156T/L317A | ++ |
| | L317A | | |
| 291/292 | K15E/G33L/M95V/V177P/Y186R/ | K15E/G33L/Y186R/D195G | ++ |
| | D195G | | |
| 293/294 | M95V/V177P/Q334R | Q334R | ++ |
| 295/296 | K15E/M95V/A154C/G156T/V177P/ | K15E/A154C/G156T/N224Y/ | ++ |
| | N224Y/L317A | L317A | |
| 297/298 | K15Y/M95V/V149R/V177P/Y186R/ | K15Y/V149R/Y186R/I331W | ++ |
| | I331W | | |
| 299/300 | K15Y/K86G/M95V/V149T/V177P/ | K15Y/K86G/V149T/Y186R/ | ++ |
| | Y186R/D195G/L317A | D195G/L317A | |
| 301/302 | K15P/G33L/M95V/A154C/V177P/ | K15P/G33L/A154C/D195G | ++ |
| | D195G | | |
| 303/304 | K15P/M95V/A154C/G156T/V177P/ | K15P/A154C/G156T/Y186R/ | ++ |
| | Y186R/L317A/I331R | L317A/I331R | |
| 305/306 | K15M/M95V/G156T/V177P/Y186R/ | K15M/G156T/Y186R/I331R | ++ |
| | I331R | | |
| 307/308 | K15Y/M95V/V149T/V177P/Y186D/ | K15Y/V149T/Y186D/L317A | ++ |
| | L317A | | |
| 309/310 | K15E/G33L/M95V/G156T/V177P/ | K15E/G33L/G156T/Y186R/ | ++ |
| | Y186R/V214I/N224Y/I331W | V214I/N224Y/I331W | |
| 311/312 | K15E/K86G/M95V/V177P/Y186R/ | K15E/K86G/Y186R/N224Y/ | ++ |
| | N224Y/K226I/L317A | K226I/L317A | |
| 313/314 | K15P/M95V/A154C/V177P/I331R/ | K15P/A154C/I331R | ++ |
| 315/316 | K15Y/M95V/V149T/A154C/V177P/ | K15Y/V149T/A154C/K226I | ++ |
| | K226I | | |
| 317/318 | K15Y/G33L/M95V/V177P/D195G/ | K15Y/G33L/D195G/K226I | ++ |
| | K226I | | |
| 319/320 | K15M/M95V/V177P/N224Y/L317A/ | K15M/N224Y/L317A/I331W | ++ |
| | I331W | | |
| 321/322 | K15E/K86G/M95V/V177P/Y186R/ | K15E/K86G/Y186R/D195G/ | ++ |
| | D195G/L317A | L317A | |
| 323/324 | M95V/V177P/K226I/L317A/I331L | K226I/L317A/I331L | ++ |
| 325/326 | M95V/V177P/T330R | T330R | ++ |
| 327/328 | K89W/M95V/V177P | K89W | ++ |
| 329/330 | K15P/M95V/V149R/V177P/Y186D/ | K15P/V149R/Y186D/I331L | ++ |
| | I331L | | |
| 331/332 | K15P/M95V/V149R/V177P/Y186R | K15P/V149R/Y186R | ++ |
| 333/334 | K89M/M95V/V177P | K89M | ++ |
| 335/336 | M95V/G156T/V177P/D195G/I331L | G156T/D195G/I331L | ++ |
| 337/338 | M95V/G156A/V177P | G156A | ++ |
| 339/340 | K15E/K86G/M95V/G156T/V177P/ | K15E/K86G/G156T/Y186R/ | ++ |
| | Y186R/I331R | I331R | |
| 341/342 | M95V/V177P/D195G/I331W | D195G/I331W | ++ |
| 343/344 | M95V/V177P/T275N | T275N | ++ |
| 345/346 | K15P/G33L/M95V/V177P/Y186R/ | K15P/G33L/Y186R/N224Y | ++ |
| | N224Y | | |
| 347/348 | K15E/M95V/V177P/Y186R/L317A | K15E/Y186R/L317A | ++ |
| 349/350 | K15M/G33L/M95V/V177P/Y186R/ | K15M/G33L/Y186R/K226I/ | ++ |
| | K226I/L317A | L317A | |
| 351/352 | K89G/M95V/V177P | K89G | + |
| 353/354 | K15Y/M95V/V149R/A154C/G156T/ | K15Y/V149R/A154C/G156T/ | + |
| | V177P/I331W | I331W | |
| 355/356 | K15Y/M95V/A154C/V177P/K226I/ | K15Y/A154C/K226I/L317A | + |
| | L317A | | |
| 357/358 | K15Y/K86G/M95V/G156T/V177P/ | K15Y/K86G/G156T/Y186R/ | + |
| | Y186R/D195G/L317A | D195G/L317A | |
| 359/360 | M95V/V177P/V257L | V257L | + |
| 361/362 | K15Y/M95V/A154C/G156T/V177P/ | K15Y/A154C/G156T/Y186D | + |
| | Y186D | | |
| 363/364 | G33L/M95V/V177P/Y186R/N224Y | G33L/Y186R/N224Y | + |
| 365/366 | M95V/V177P/N335H | N335H | + |
| 367/368 | K15Y/K86G/M95V/G156T/V177P/ | K15Y/K86G/G156T/Y186R/ | + |
| | Y186R/D195G/N224Y/I331W | D195G/N224Y/I331W | |
| 369/370 | M95V/A154C/G156T/V177P/L317A | A154C/G156T/L317A | + |
| 371/372 | H39Q/M95V/V177P | H39Q | + |
| 373/374 | H2G/M95V/V177P | H2G | + |
| 375/376 | K15M/M95V/V177P/N224Y/K226I/ | K15M/N224Y/K226I/I331R | + |
| | I331R | | |
| 377/378 | K15M/G33L/M95V/V149R/V177P/ | K15M/G33L/V149R/N224Y | + |
| | N224Y | | |
| 379/380 | K89I/M95V/V177P/N307Q | K89I/N307Q | + |
| 381/382 | K15M/M95V/A154C/G156T/V177P/ | K15M/A154C/G156T/L317A | + |
| | L317A | | |
| 383/384 | K15E/M95V/V177P/Y186R/I331W | K15E/Y186R/I331W | + |
| 385/386 | K15P/G33L/M95V/A154C/V177P/ | K15P/G33L/A154C/L317A | + |
| | L317A | | |
| 387/388 | K89H/M95V/V177P | K89H | + |
| 389/390 | K15Y/G33L/M95V/V177P/D195G/ | K15Y/G33L/D195G/N224Y/ | + |
| | N224Y/L317A | L317A | |
| 391/392 | K15E/M95V/G156T/V177P/N224Y/ | K15E/G156T/N224Y/K226I | + |
| | K226I | | |
| 393/394 | M95V/A154C/V177P/Y186R/I331L | A154C/Y186R/I331L | + |
| 395/396 | M95V/V177P/N185G | N185G | + |
| 397/398 | K15M/S18I/M95V/V149R/V177P/ | K15M/S18I/V149R/Y186R/ | + |
| | Y186R/D195G | D195G | |
| 399/400 | K86G/M95V/V149R/A154C/G156T/ | K86G/V149R/A154C/G156T | + |
| | V177P | | |
| 401/402 | K15P/G33L/M95V/V149R/V177P/ | K15P/G33L/V149R/I331L | + |
| | I331L | | |
| 403/404 | M95V/V177P/K326R | K326R | + |
| 405/406 | M95V/V177P/H202Y | H202Y | + |
| 407/408 | M95V/Q119T/V177P | Q119T | + |
| 409/410 | M95V/V177P/T212I | T212I | + |
| 411/412 | H2V/M95V/V177P | H2V | + |
| 413/414 | K15E/M95V/V149R/A154C/G156T/ | K15E/V149R/A154C/G156T/ | + |
| | V177P/I331L | I331L | |
| 415/416 | K15P/G33L/M95V/G156T/V177P/ | K15P/G33L/G156T/L317A | + |
| | L317A | | |
| 417/418 | K15Y/M95V/A154C/G156T/V177P/ | K15Y/A154C/G156T/L317A | + |
| | L317A | | |
| 419/420 | M95V/G156L/V177P | G156L | + |
| 421/422 | M95V/V177P/N335D | N335D | + |
| 423/424 | M95V/G156T/V177P | G156T | + |
| 425/426 | H2E/M95V/V177P | H2E | + |
| 427/428 | M95V/V177P/S280G | S280G | + |
| 429/430 | M95V/K142R/V177P | K142R | + |
| 431/432 | K15E/M95V/V149R/V177P | K15E/V149R | + |
| 433/434 | M95V/G156Q/V177P | G156Q | + |
| 435/436 | K15E/G33L/K86G/M95V/G156T/ | K15E/G33L/K86G/G156T/ | + |
| | V177P/D195G | D195G | |
| 437/438 | M95V/V177P/D315T | D315T | + |
| 439/440 | M95V/V177P/N185K | N185K | + |
| 441/442 | H39S/M95V/V177P | H39S | + |
| 443/444 | M95V/V177P/K256A | K256A | + |
| 445/446 | K15Y/G33L/M95V/G156T/V177P/ | K15Y/G33L/G156T/Y186D | + |
| | Y186D | | |
| 447/448 | M95V/S138R/V177P | S138R | + |
| 449/450 | M95V/G117D/V177P | G117D | + |
| 451/452 | M95V/G116N/V177P | G116N | + |
| 453/454 | M95V/V177P/M316L | M316L | + |
| 455/456 | K15M/M95V/V149R/V177P | K15M/V149R | + |
| 457/458 | T69D/M95V/V177P | T69D | + |
| 459/460 | M95V/G156N/V177P | G156N | + |
| 461/462 | M95V/V177P/A310K | A310K | + |
| 463/464 | M95V/V177P/L317A | L317A | + |
| 465/466 | K15Y/M95V/V177P/D195G/I331L | K15Y/D195G/I331L | + |
| 467/468 | K15M/G33L/M95V/V177P/I331L | K15M/G33L/I331L | + |
| 469/470 | M95V/V177P/T330M | T330M | + |
| 471/472 | M95V/V177P/H314W | H314W | + |
| 473/474 | M95V/Y144W/V177P | Y144W | |
| 475/476 | K15Y/M95V/V149R/V177P | K15Y/V149R | + |
| 477/478 | M95V/V177P/D315S | D315S | + |

Levels of increased activity were determined relative to the reference polypeptide of SEQ ID NO: 42 and defined as follows: “+” 2.00 to 3.82 (first 50%), “++” > 3.82 (next 30%), “+++” > 8.29 (top 20%)

### Example 7

### Improvements Over SEQ ID NO: 128 in RNA Ligase 2 Activity

SEQ ID NO: 42 was recloned into a pJV vector to give SEQ ID NO: 128, which was further selected as the parent enzyme. Libraries of engineered genes were produced using established techniques (e.g., saturation mutagenesis, recombination of previously identified beneficial mutations). The polypeptides encoded by each gene were produced in HIP as described in Example 1 and the cell paste was generated as described in Example 2.

To prepare cells for lysis, 300 μl of 0.1 mg/mL lysozyme in 50 mM Tris-HCl buffer at pH 7.5 was added to the cell paste of each sample. After thorough resuspension, the cells were incubated at 45° C. for 1 hour in a PCR thermocycler. The samples were then centrifuged for 10 minutes at 4000 rpm and 4° C. and the clear supernatants used in subsequent biocatalytic reactions.

To screen, 20%(v/v) lysate was transferred into reactions containing 50 mM Tris-HCl buffer at pH 7.5, 2 mM MgCl₂, 1 mM DTT, 0.4 mM ATP, and 0.4 mM of an equimolar mixture of the fragments compromising Reaction 2 (sense strands 1-3, and antisense strands 1-2). The reactions were mixed before being incubated for 16 hours at 37° C. in a PCR thermocycler. Afterwards, EDTA was added to each reaction to a final concentration of 18.3 mM and the samples analyzed via UPLC to quantify the remaining fragment concentrations and product yields.

Using peak area from Analytical method 13.1, the % conversion for each sample was calculated as shown here:

$$\% conversion = \frac{area under the curve for all products}{\begin{matrix}Total area under the curve for all \\ products and remaining fragments\end{matrix}} * 100$$

Activity relative to SEQ ID NO: 128 (FIOP) was calculated as the % conversion formed by the variant over % conversion of SEQ ID NO: 128 and shown in Table 7.1.

TABLE 7.1
Relative to SEQ ID NO: 128

| SEQ | Amino Acid | Amino Acid | FIOP |
|---|---|---|---|
| ID | Differences | Differences | Relative to |
| NO: | (Relative to | (Relative to | SEQ ID |
| (nt/aa) | SEQ ID NO: 14) | SEQ ID NO: 128) | NO: 128 |
| 129/130 | K15M/M95V/V149R/A154C/G156T/ | K15M/V149R/A154C/G156T/ | +++ |
| | V177P/Y186R/D195G/I331R | Y186R/D195G/I331R | |
| 131/132 | M95V/V149R/A154C/V177P/Y186D/ | V149R/A154C/Y186D/D195G/ | +++ |
| | D195G/N224Y/L317A/I331R | N224Y/L317A/I331R | |
| 133/134 | K15M/G33L/M95V/V149T/A154C/ | K15M/G33L/V149T/A154C/ | +++ |
| | V177P/Y186R/D195G | Y186R/D195G | |
| 135/136 | M95V/V149R/G156T/V177P/Y186R/ | V149R/G156T/Y186R/D195G/ | +++ |
| | D195G/N224Y/K226I/L317A | N224Y/K226I/L317A | |
| 137/138 | K15Y/M95V/V149R/G156T/V177P/ | K15Y/V149R/G156T/D195G/ | +++ |
| | D195G/N224Y/K226I | N224Y/K226I | |
| 139/140 | K15Y/G33L/K86G/M95V/V149T/ | K15Y/G33L/K86G/V149T/ | +++ |
| | A154C/G156T/V177P/Y186R/D195G/ | A154C/G156T/Y186R/D195G/ | |
| | K2261/1331W | K226I/I331W | |
| 141/142 | K15Y/M95V/V149R/G156T/V177P/ | K15Y/V149R/G156T/Y186R/ | +++ |
| | Y186R/D195G/N224Y/L317A/I331W | D195G/N224Y/L317A/I331W | |
| 143/144 | K15M/G33L/M95V/V149T/A154C/ | K15M/G33L/V149T/A154C/ | +++ |
| | V177P/D195G/L317A | D195G/L317A | |
| 145/146 | K15M/M95V/V149R/A154C/V177P/ | K15M/V149R/A154C/D195G/ | +++ |
| | D195G/N224Y/L317A | N224Y/L317A | |
| 147/148 | K15Y/M95V/V149R/G156T/V177P/ | K15Y/V149R/G156T/Y186D/ | ++ |
| | Y186D/N224Y/K226I/L317A/I331R | N224Y/K226I/L317A/I331R | |
| 149/150 | K15M/M95V/V149T/A154C/G156T/ | K15M/V149T/A154C/G156T/ | ++ |
| | V177P/Y186D/D195G/I331L | Y186D/D195G/I331L | |
| 151/152 | K15Y/G33L/M95V/V149R/A154C/ | K15Y/G33L/V149R/A154C/ | ++ |
| | G156T/V177P/Y186R/D195G/N224Y/ | G156T/Y186R/D195G/N224Y/ | |
| | K226I/L317A/I331R | K226I/L317A/I331R | |
| 153/154 | M95V/V149R/V177P/D195G/N224Y | V149R/D195G/N224Y | ++ |
| 155/156 | K86G/M95V/V149T/A154C/G156T/ | K86G/V149T/A154C/G156T/ | ++ |
| | V177P/Y186R/D195G/I331L | Y186R/D195G/I331L | |
| 157/158 | K15P/G33L/M95V/V149R/G156T/ | K15P/G33L/V149R/G156T/ | ++ |
| | V177P/D195G/N224Y/L317A/I331L | D195G/N224Y/L317A/I331L | |
| 159/160 | K15P/M95V/V149T/A154C/G156T/ | K15P/V149T/A154C/G156T/Y | ++ |
| | V177P/Y186D/D195G/L317A/I331L | 186D/D195G/L317A/I331L | |
| 161/162 | K15Y/G33L/M95V/V149T/A154C/G | K15Y/G33L/V149T/A154C/G | ++ |
| | 156T/V177P/Y186D/N224Y/I331L | 156T/Y186D/N224Y/I331L | |
| 163/164 | K15P/M95V/V149R/G156T/V177P/ | K15P/V149R/G156T/Y186R/ | ++ |
| | Y186R/D195G/N224Y/L317A/I331W | D195G/N224Y/L317A/I331W | |
| 165/166 | K15Y/M95V/V149R/A154C/G156T/ | K15Y/V149R/A154C/G156T/ | ++ |
| | V177P/D195G/N224Y | D195G/N224Y | |
| 167/168 | K15M/M95V/V149T/A154C/G156T/ | K15M/V149T/A154C/G156T/ | ++ |
| | V177P/D195G/N224Y/K226I | D195G/N224Y/K226I | |
| 169/170 | K15E/M95V/V149R/A154C/G156T/ | K15E/V149R/A154C/G156T/ | ++ |
| | V177P/Y186R/D195G/N224Y/L317A | Y186R/D195G/N224Y/L317A | |
| 171/172 | G33L/M95V/V149T/G156T/V177P/ | G33L/V149T/G156T/Y186R/ | ++ |
| | Y186R/N224Y/I331L | N224Y/I331L | |
| 173/174 | K15Y/G33L/K86G/M95V/V149R/ | K15Y/G33L/K86G/V149R/ | ++ |
| | V177P/D195G/N224Y/I331W | D195G/N224Y/I331W | |
| 175/176 | K15E/K86G/M95V/V149R/A154C/ | K15E/K86G/V149R/A154C/ | + |
| | G156T/V177P/Y186R/D195G/I331W | G156T/Y186R/D195G/I331W | |
| 177/178 | M95V/V149R/V177P/Y186R/D195G/ | V149R/Y186R/D195G/I331W | + |
| | I331W | | |
| 179/180 | K15E/M95V/A154C/G156T/V177P/ | K15E/A154C/G156T/Y186D/ | + |
| | Y186D/K226I/I331R | K226I/I331R | |
| 181/182 | K15Y/M95V/V149T/A154C/V177P/ | K15Y/V149T/A154C/Y186R/ | + |
| | Y186R/D195G/N224Y/K226I | D195G/N224Y/K226I | |
| 183/184 | K15P/M95V/V149R/A154C/G156T/ | K15P/V149R/A154C/G156T/ | + |
| | V177P/D195G/N224Y/I331W | D195G/N224Y/I331W | |
| 185/186 | K15E/G33L/M95V/V149R/A154C/ | K15E/G33L/V149R/A154C/ | + |
| | G156T/V177P/D195G/L317A/I331L | G156T/D195G/L317A/I331L | |
| 187/188 | K15E/G33L/M95V/A154C/G156T/ | K15E/G33L/A154C/G156T/ | + |
| | V177P/Y186R/D195G/N224Y/L317A/ | Y186R/D195G/N224Y/L317A/ | |
| | I331L | I331L | |
| 189/190 | K15P/M95V/V149R/V177P/Y186D/ | K15P/V149R/Y186D/D195G/ | + |
| | D195G/N224Y/I331L | N224Y/I331L | |
| 191/192 | K15P/G33L/M95V/V149R/A154C/ | K15P/G33L/V149R/A154C/ | + |
| | G156T/V177P/D195G/N224Y/I331W | G156T/D195G/N224Y/I331W | |
| 193/194 | K15P/M95V/V149T/A154C/G156T/ | K15P/V149T/A154C/G156T/ | + |
| | V177P/N224Y/K226I/L317A/I331W | N224Y/K226I/L317A/I331W | |
| 195/196 | K15P/G33L/M95V/V149T/G156T/ | K15P/G33L/V149T/G156T/ | + |
| | V177P/D195G/I331W | D195G/I331W | |
| 197/198 | K15P/M95V/V149T/A154C/G156T/ | K15P/V149T/A154C/G156T/ | + |
| | V177P/Y186D/N224Y/K226I/I331L | Y186D/N224Y/K226I/I331L | |
| 199/200 | M95V/A154C/G156T/V177P/D195G/ | A154C/G156T/D195G/N224Y/ | + |
| | N224Y/K226I | K226I | |
| 201/202 | K15Y/G33L/K86G/M95V/V149R/ | K15Y/G33L/K86G/V149R/ | + |
| | A154C/G156T/V177P/Y186D/D195G/ | A154C/G156T/Y186D/D195G/ | |
| | N224Y/L317A/I331W | N224Y/L317A/I331W | |
| 203/204 | K15Y/M95V/V149R/A154C/G156T/ | K15Y/V149R/A154C/G156T/ | + |
| | V177P/Y186R/D195G/N224Y/K226I | Y186R/D195G/N224Y/K226I | |
| 205/206 | K15P/G33L/M95V/V149R/V177P/ | K15P/G33L/V149R/D195G/ | + |
| | D195G/L317A/I331L | L317A/I331L | |
| 207/208 | K15E/M95V/V149T/A154C/V177P/ | K15E/V149T/A154C/N224Y/ | + |
| | N224Y/I331R | I331R | |
| 209/210 | M95V/V149T/A154C/G156T/V177P/ | V149T/A154C/G156T/D195G/ | + |
| | D195G/N224Y/K226I/I331R | N224Y/K226I/I331R | |
| 211/212 | K15M/M95V/V149R/A154C/V177P/ | K15M/V149R/A154C/D195G/ | + |
| | D195G/I331L | I331L | |
| 213/214 | K15Y/M95V/V149R/A154C/G156T/ | K15Y/V149R/A154C/G156T/ | + |
| | V177P/Y186R/D195G | Y186R/D195G | |
| 215/216 | K15E/M95V/V149T/A154C/G156T/ | K15E/V149T/A154C/G156T/ | + |
| | V177P/Y186R/D195G/I331R | Y186R/D195G/I331R | |
| 217/218 | K15M/G33L/M95V/V149T/A154C/ | K15M/G33L/V149T/A154C/ | + |
| | G156T/V177P/Y186R/D195G | G156T/Y186R/D195G | |
| 219/220 | K15P/G33L/K86G/M95V/V149R/ | K15P/G33L/K86G/V149R/ | + |
| | A154C/V177P/D195G/N224Y/L317A/ | A154C/D195G/N224Y/L317A/ | |
| | I331R | I331R | |
| 221/222 | M95V/V149R/V177P/Y186D/D195G/ | V149R/Y186D/D195G/N224Y/ | + |
| | N224Y/K226I/I331R | K226I/I331R | |

Levels of increased activity were determined relative to the reference polypeptide of SEQ ID NO: 128 and defined as follows: “+” 18.99 to 22.90 (first 50%), “++” > 22.90 (next 30%), “+++” > 25.50 (top 20%)

### Example 8

### Improvements Over SEQ ID NO: 128 in RNA Ligase 2 Activity

SEQ ID NO: 128 was selected as the parent enzyme after screening variants as described in Example 5 above, lop variants were selected from Example 7 above to test again under another condition. Libraries of engineered genes were produced using established techniques (e.g., saturation mutagenesis, recombination of previously identified beneficial mutations). The polypeptides encoded by each gene were produced in HIP as described in Example 1 and the cell paste was generated as described in Example 2.

To prepare cells for lysis, 300 μl of 0.1 mg/mL lysozyme in 50 mM Tris-HCl buffer at pH 7.5 was added to the cell paste of each sample. After thorough resuspension, the cells were incubated at 45° C. for 1 hour in a PCR thermocycler. The samples were then centrifuged for 10 minutes at 4000 rpm and 4° C. and the clear supernatants used in subsequent biocatalytic reactions.

To screen, 20% (v/v) lysate was transferred into reactions containing 50 mM Tris-HCl buffer at pH 7.5, 2 mM MgCl₂, 1 mM DTT, 0.4 mM ATP, and 0.4 mM of an equimolar mixture of the fragments compromising Reaction 1 (sense strands 1, 4, and 5, and antisense strands 1-2). The reactions were mixed before being incubated for 16 hours at 37° C. in a PCR thermocycler. Afterwards, EDTA was added to each reaction to a final concentration of 18.3 mM and the samples analyzed via UPLC to quantify the remaining fragment concentrations and product yields.

Using peak area from Analytical method 13.1, the % conversion for each sample was calculated as shown here:

$$\% conversion = \frac{area under the curve for all products}{\begin{matrix}Total area under the curve for all \\ products and remaining fragments\end{matrix}} * 100$$

Activity relative to SEQ ID NO: 128 (FIOP) was calculated as the % conversion formed by the variant over % conversion of SEQ ID NO: 128 and shown in Table 8.1.

TABLE 8.1
Relative to SEQ ID NO: 128

| SEQ | Amino Acid | Amino Acid | FIOP |
|---|---|---|---|
| ID | Differences | Differences | Relative to |
| NO: | (Relative to | (Relative to | SEQ ID |
| (nt/aa) | SEQ ID NO: 14) | SEQ ID NO: 128) | NO: 128 |
| 209/210 | M95V/V149T/A154C/G156T/V177P/ | V149T/A154C/G156T/D195G/ | +++ |
| | D195G/N224Y/K226I/I331R | N224Y/K226I/I331R | |
| 151/152 | K15Y/G33L/M95V/V149R/A154C/ | K15Y/G33L/V149R/A154C/G156T/ | +++ |
| | G156T/V177P/Y186R/D195G/ | Y186R/D195G/N224Y/K226I/ | |
| | N224Y/K226I/L317A/I331R | L317A/I331R | |
| 201/202 | K15Y/G33L/K86G/M95V/V149R/ | K15Y/G33L/K86G/V149R/A154C/ | +++ |
| | A154C/G156T/V177P/Y186D/ | G156T/Y186D/D195G/N224Y/ | |
| | D195G/N224Y/L317A/I331W | L317A/I331W | |
| 131/132 | M95V/V149R/A154C/V177P/ | V149R/A154C/Y186D/D195G/ | +++ |
| | Y186D/D195G/N224Y/L317A/I331R | N224Y/L317A/I331R | |
| 135/136 | M95V/V149R/G156T/V177P/ | V149R/G156T/Y186R/D195G/ | +++ |
| | Y186R/D195G/N224Y/K226I/L317A | N224Y/K226I/L317A | |
| 163/164 | K15P/M95V/V149R/G156T/ | K15P/V149R/G156T/Y186R/ | +++ |
| | V177P/Y186R/D195G/N224Y/ | D195G/N224Y/L317A/I331W | |
| | L317A/I331W | | |
| 203/204 | K15Y/M95V/V149R/A154C/G156T/ | K15Y/V149R/A154C/G156T/ | +++ |
| | V177P/Y186R/D195G/N224Y/ | Y186R/D195G/N224Y/K226I | |
| | K226I | | |
| 221/222 | M95V/V149R/V177P/Y186D/ | V149R/Y186D/D195G/N224Y/ | +++ |
| | D195G/N224Y/K226I/I331R | K226I/I331R | |
| 129/130 | K15M/M95V/V149R/A154C/G156T/ | K15M/V149R/A154C/G156T/ | +++ |
| | V177P/Y186R/D195G/I331R | Y186R/D195G/I331R | |
| 181/182 | K15Y/M95V/V149T/A154C/V177P/ | K15Y/V149T/A154C/Y186R/ | ++ |
| | Y186R/D195G/N224Y/K226I | D195G/N224Y/K226I | |
| 157/158 | K15P/G33L/M95V/V149R/G156T/ | K15P/G33L/V149R/G156T/ | ++ |
| | V177P/D195G/N224Y/L317A/ | D195G/N224Y/L317A/I331L | |
| | I331L | | |
| 141/142 | K15Y/M95V/V149R/G156T/ | K15Y/V149R/G156T/Y186R/ | ++ |
| | V177P/Y186R/D195G/N224Y/ | D195G/N224Y/L317A/I331W | |
| | L317A/I331W | | |
| 167/168 | K15M/M95V/V149T/A154C/G156T/ | K15M/V149T/A154C/G156T/ | ++ |
| | V177P/D195G/N224Y/K226I | D195G/N224Y/K226I | |
| 215/216 | K15E/M95V/V149T/A154C/G156T/ | K15E/V149T/A154C/G156T/ | ++ |
| | V177P/Y186R/D195G/I331R | Y186R/D195G/I331R | |
| 183/184 | K15P/M95V/V149R/A154C/G156T/ | K15P/V149R/A154C/G156T/ | ++ |
| | V177P/D195G/N224Y/I331W | D195G/N224Y/I331W | |
| 169/170 | K15E/M95V/V149R/A154C/G156T/ | K15E/V149R/A154C/G156T/ | ++ |
| | V177P/Y186R/D195G/N224Y/ | Y186R/D195G/N224Y/L317A | |
| | L317A | | |
| 165/166 | K15Y/M95V/V149R/A154C/G156T/ | K15Y/V149R/A154C/G156T/ | ++ |
| | V177P/D195G/N224Y | D195G/N224Y | |
| 159/160 | K15P/M95V/V149T/A154C/G156T/ | K15P/V149T/A154C/G156T/ | ++ |
| | V177P/Y186D/D195G/L317A/ | Y186D/D195G/L317A/I331L | |
| | I331L | | |
| 149/150 | K15M/M95V/V149T/A154C/ | K15M/V149T/A154C/G156T/ | ++ |
| | G156T/V177P/Y186D/D195G/I331L | Y186D/D195G/I331L | |
| 217/218 | K15M/G33L/M95V/V149T/A154C/ | K15M/G33L/V149T/A154C/ | ++ |
| | G156T/V177P/Y186R/D195G | G156T/Y186R/D195G | |
| 213/214 | K15Y/M95V/V149R/A154C/ | K15Y/V149R/A154C/G156T/Y186R/ | ++ |
| | G156T/V177P/Y186R/D195G | D195G | |
| 137/138 | K15Y/M95V/V149R/G156T/ | K15Y/V149R/G156T/D195G/N224Y/ | ++ |
| | V177P/D195G/N224Y/K226I | K226I | |
| 219/220 | K15P/G33L/K86G/M95V/V149R/ | K15P/G33L/K86G/V149R/A154C/ | ++ |
| | A154C/V177P/D195G/N224Y/ | D195G/N224Y/L317A/I331R | |
| | L317A/I331R | | |
| 191/192 | K15P/G33L/M95V/V149R/A154C/ | K15P/G33L/V149R/A154C/G156T/ | + |
| | G156T/V177P/D195G/N224Y/ | D195G/N224Y/I331W | |
| | I331W | | |
| 189/190 | K15P/M95V/V149R/V177P/ | K15P/V149R/Y186D/D195G/N224Y/ | + |
| | Y186D/D195G/N224Y/I331L | I331L | |
| 133/134 | K15M/G33L/M95V/V149T/ | K15M/G33L/V149T/A154C/Y186R/ | + |
| | A154C/V177P/Y186R/D195G | D195G | |
| 161/162 | K15Y/G33L/M95V/V149T/A154C/ | K15Y/G33L/V149T/A154C/G156T/ | + |
| | G156T/V177P/Y186D/N224Y/ | Y186D/N224Y/I331L | |
| | I331L | | |
| 185/186 | K15E/G33L/M95V/V149R/A154C/ | K15E/G33L/V149R/A154C/G156T/ | + |
| | G156T/V177P/D195G/L317A/ | D195G/L317A/I331L | |
| | I331L | | |
| 139/140 | K15Y/G33L/K86G/M95V/V149T/ | K15Y/G33L/K86G/V149T/A154C/ | + |
| | A154C/G156T/V177P/Y186R/ | G156T/Y186R/D195G/K226I/I331W | |
| | D195G/K226I/I331W | | |
| 177/178 | M95V/V149R/V177P/Y186R/ | V149R/Y186R/D195G/I331W | + |
| | D195G/I331W | | |
| 143/144 | K15M/G33L/M95V/V149T/ | K15M/G33L/V149T/A154C/ | + |
| | A154C/V177P/D195G/L317A | D195G/L317A | |
| 187/188 | K15E/G33L/M95V/A154C/G156T/ | K15E/G33L/A154C/G156T/ | + |
| | V177P/Y186R/D195G/N224Y/ | Y186R/D195G/N224Y/L317A/I331L | |
| | L317A/I331L | | |
| 145/146 | K15M/M95V/V149R/A154C/ | K15M/V149R/A154C/D195G/ | + |
| | V177P/D195G/N224Y/L317A | N224Y/L317A | |
| 147/148 | K15Y/M95V/V149R/G156T/V177P/ | K15Y/V149R/G156T/Y186D/ | + |
| | Y186D/N224Y/K226I/L317A/ | N224Y/K226I/L317A/I331R | |
| | I331R | | |
| 195/196 | K15P/G33L/M95V/V149T/G156T/ | K15P/G33L/V149T/G156T/D195G/ | + |
| | V177P/D195G/I331W | I331W | |
| 153/154 | M95V/V149R/V177P/D195G/ | V149R/D195G/N224Y | + |
| | N224Y | | |
| 211/212 | K15M/M95V/V149R/A154C/ | K15M/V149R/A154C/D195G/I331L | + |
| | V177P/D195G/I331L | | |
| 193/194 | K15P/M95V/V149T/A154C/G156T/ | K15P/V149T/A154C/G156T/N224Y/ | + |
| | V177P/N224Y/K226I/L317A/ | K226I/L317A/I331W | |
| | I331W | | |
| 197/198 | K15P/M95V/V149T/A154C/G156T/ | K15P/V149T/A154C/G156T/Y186D/ | + |
| | V177P/Y186D/N224Y/K226I/ | N224Y/K226I/I331L | |
| | I331L | | |
| 171/172 | G33L/M95V/V149T/G156T/ | G33L/V149T/G156T/Y186R/ | + |
| | V177P/Y186R/N224Y/I331L | N224Y/I331L | |
| 155/156 | K86G/M95V/V149T/A154C/G156T/ | K86G/V149T/A154C/G156T/ | + |
| | V177P/Y186R/D195G/I331L | Y186R/D195G/I331L | |
| 199/200 | M95V/A154C/G156T/V177P/ | A154C/G156T/D195G/N224Y/ | + |
| | D195G/N224Y/K226I | K226I | |
| 173/174 | K15Y/G33L/K86G/M95V/V149R/ | K15Y/G33L/K86G/V149R/D195G/ | + |
| | V177P/D195G/N224Y/I331W | N224Y/I331W | |
| 205/206 | K15P/G33L/M95V/V149R/V177P/ | K15P/G33L/V149R/D195G/L317A/ | + |
| | D195G/L317A/I331L | I331L | |
| 175/176 | K15E/K86G/M95V/V149R/A154C/ | K15E/K86G/V149R/A154C/G156T/ | + |
| | G156T/V177P/Y186R/D195G/ | Y186R/D195G/I331W | |
| | I331W | | |
| 207/208 | K15E/M95V/V149T/A154C/ | K15E/V149T/A154C/N224Y/I331R | + |
| | V177P/N224Y/I331R | | |
| 179/180 | K15E/M95V/A154C/G156T/V177P/ | K15E/A154C/G156T/Y186D/K226I/ | + |
| | Y186D/K226I/I331R | I331R | |

Levels of increased activity were determined relative to the reference polypeptide of SEQ ID NO: 128 and defined as follows: “+” 11.21 to 19.80 (first 50%), “++” > 19.80 (next 30%), “+++” > 20.97 (top 20%)

### Example 9

### Improvements Over SEQ ID NO: 128 in RNA Ligase 2 Activity

The same samples from Example 8 were tested again, but with Reaction 3. To screen, 20%(v/v) lysate was transferred into reactions containing 50 mM Tris-HCl buffer at pH 7.5, 2 mM MgCl₂, 1 mM DTT, 0.4 mM ATP, and 0.4 mM of an equimolar mixture of the fragments compromising Reaction 3 (sense strands 1, 4 and 5, and antisense strands 2-4). The reactions were mixed before being incubated for 16 hours at 37° C. in a PCR thermocycler. Afterwards, EDTA was added to each reaction to a final concentration of 18.3 mM and the samples analyzed via UPLC to quantify the remaining fragment concentrations and product yields.

Using peak area from Analytical method 13.1, the % conversion for each sample was calculated as shown here:

$$\% conversion = \frac{area under the curve for all products}{\begin{matrix}Total area under the curve for all \\ products and remaining fragments\end{matrix}} * 100$$

Activity relative to SEQ ID NO: 128 (FIOP) was calculated as the % conversion formed by the variant over % conversion of SEQ ID NO: 128 and shown in Table 9.1.

TABLE 9.1
Relative to SEQ ID NO: 128

| SEQ | Amino Acid | Amino Acid | FIOP |
|---|---|---|---|
| ID | Differences | Differences | Relative to |
| NO: | (Relative to | (Relative to | SEQ ID |
| (nt/aa) | SEQ ID NO: 14) | SEQ ID NO: 40 and 128) | NO: 128 |
| 163/164 | K15P/M95V/V149R/G156T/V177P/ | K15P/V149R/G156T/Y186R/ | +++ |
| | Y186R/D195G/N224Y/L317A/I331W | D195G/N224Y/L317A/I331W | |
| 203/204 | K15Y/M95V/V149R/A154C/G156T/ | K15Y/V149R/A154C/G156T/ | +++ |
| | V177P/Y186R/D195G/N224Y/K226I | Y186R/D195G/N224Y/K226I | |
| 141/142 | K15Y/M95V/V149R/G156T/V177P/ | K15Y/V149R/G156T/Y186R/ | +++ |
| | Y186R/D195G/N224Y/L317A/I331W | D195G/N224Y/L317A/I331W | |
| 131/132 | M95V/V149R/A154C/V177P/Y186D/ | V149R/A154C/Y186D/D195G/ | +++ |
| | D195G/N224Y/L317A/I331R | N224Y/L317A/I331R | |
| 169/170 | K15E/M95V/V149R/A154C/G156T/ | K15E/V149R/A154C/G156T/ | +++ |
| | V177P/Y186R/D195G/N224Y/L317A | Y186R/D195G/N224Y/L317A | |
| 201/202 | K15Y/G33L/K86G/M95V/V149R/ | K15Y/G33L/K86G/V149R/ | +++ |
| | A154C/G156T/V177P/Y186D/D195G/ | A154C/G156T/Y186D/D195G/ | |
| | N224Y/L317A/I331W | N224Y/L317A/I331W | |
| 191/192 | K15P/G33L/M95V/V149R/A154C/ | K15P/G33L/V149R/A154C/ | +++ |
| | G156T/V177P/D195G/N224Y/I331W | G156T/D195G/N224Y/I331W | |
| 183/184 | K15P/M95V/V149R/A154C/G156T/ | K15P/V149R/A154C/G156T/ | +++ |
| | V177P/D195G/N224Y/I331W | D195G/N224Y/I331W | |
| 157/158 | K15P/G33L/M95V/V149R/G156T/ | K15P/G33L/V149R/G156T/ | +++ |
| | V177P/D195G/N224Y/L317A/I331L | D195G/N224Y/L317A/I331L | |
| 219/220 | K15P/G33L/K86G/M95V/V149R/A154C/ | K15P/G33L/K86G/V149R/ | ++ |
| | V177P/D195G/N224Y/L317A/I331R | A154C/D195G/N224Y/L317A/I331R | |
| 151/152 | K15Y/G33L/M95V/V149R/A154C/ | K15Y/G33L/V149R/A154C/ | ++ |
| | G156T/V177P/Y186R/D195G/N224Y/ | G156T/Y186R/D195G/N224Y/ | |
| | K226I/L317A/I331R | K226I/L317A/I331R | |
| 167/168 | K15M/M95V/V149T/A154C/G156T/ | K15M/V149T/A154C/G156T/ | ++ |
| | V177P/D195G/N224Y/K226I | D195G/N224Y/K226I | |
| 129/130 | K15M/M95V/V149R/A154C/G156T/ | K15M/V149R/A154C/G156T/ | ++ |
| | V177P/Y186R/D195G/I331R | Y186R/D195G/I331R | |
| 221/222 | M95V/V149R/V177P/Y186D/D195G/ | V149R/Y186D/D195G/N224Y/ | ++ |
| | N224Y/K226I/I331R | K226I/I331R | |
| 189/190 | K15P/M95V/V149R/V177P/Y186D/ | K15P/V149R/Y186D/D195G/ | ++ |
| | D195G/N224Y/I331L | N224Y/I331L | |
| 181/182 | K15Y/M95V/V149T/A154C/V177P/ | K15Y/V149T/A154C/Y186R/ | ++ |
| | Y186R/D195G/N224Y/K226I | D195G/N224Y/K226I | |
| 165/166 | K15Y/M95V/V149R/A154C/G156T/ | K15Y/V149R/A154C/G156T/ | ++ |
| | V177P/D195G/N224Y | D195G/N224Y | |
| 159/160 | K15P/M95V/V149T/A154C/G156T/ | K15P/V149T/A154C/G156T/ | ++ |
| | V177P/Y186D/D195G/L317A/I331L | Y186D/D195G/L317A/I331L | |
| 135/136 | M95V/V149R/G156T/V177P/Y186R/ | V149R/G156T/Y186R/D195G/ | ++ |
| | D195G/N224Y/K226I/L317A | N224Y/K226I/L317A | |
| 209/210 | M95V/V149T/A154C/G156T/V177P/ | V149T/A154C/G156T/D195G/ | ++ |
| | D195G/N224Y/K226I/I331R | N224Y/K226I/I331R | |
| 217/218 | K15M/G33L/M95V/V149T/A154C/ | K15M/G33L/V149T/A154C/ | ++ |
| | G156T/V177P/Y186R/D195G | G156T/Y186R/D195G | |
| 197/198 | K15P/M95V/V149T/A154C/G156T/ | K15P/V149T/A154C/G156T/ | ++ |
| | V177P/Y186D/N224Y/K226I/I331L | Y186D/N224Y/K226I/I331L | |
| 193/194 | K15P/M95V/V149T/A154C/G156T/ | K15P/V149T/A154C/G156T/ | ++ |
| | V177P/N224Y/K226I/L317A/I331W | N224Y/K226I/L317A/I331W | |
| 149/150 | K15M/M95V/V149T/A154C/G156T/ | K15M/V149T/A154C/G156T/ | + |
| | V177P/Y186D/D195G/I331L | Y186D/D195G/I331L | |
| 215/216 | K15E/M95V/V149T/A154C/G156T/ | K15E/V149T/A154C/G156T/ | + |
| | V177P/Y186R/D195G/I331R | Y186R/D195G/I331R | |
| 137/138 | K15Y/M95V/V149R/G156T/V177P/ | K15Y/V149R/G156T/D195G/ | + |
| | D195G/N224Y/K226I | N224Y/K226I | |
| 145/146 | K15M/M95V/V149R/A154C/V177P/ | K15M/V149R/A154C/D195G/ | + |
| | D195G/N224Y/L317A | N224Y/L317A | |
| 139/140 | K15Y/G33L/K86G/M95V/V149T/ | K15Y/G33L/K86G/V149T/ | + |
| | A154C/G156T/V177P/Y186R/D195G/ | A154C/G156T/Y186R/D195G/ | |
| | K226I/I331W | K226I/I331W | |
| 187/188 | K15E/G33L/M95V/A154C/G156T/ | K15E/G33L/A154C/G156T/ | + |
| | V177P/Y186R/D195G/N224Y/L317A/ | Y186R/D195G/N224Y/L317A/ | |
| | I331L | I331L | |
| 153/154 | M95V/V149R/V177P/D195G/N224Y | V149R/D195G/N224Y | + |
| 185/186 | K15E/G33L/M95V/V149R/A154C/ | K15E/G33L/V149R/A154C/ | + |
| | G156T/V177P/D195G/L317A/I331L | G156T/D195G/L317A/I331L | |
| 213/214 | K15Y/M95V/V149R/A154C/G156T/ | K15Y/V149R/A154C/G156T/ | + |
| | V177P/Y186R/D195G | Y186R/D195G | |
| 171/172 | G33L/M95V/V149T/G156T/V177P/ | G33L/V149T/G156T/Y186R/ | + |
| | Y186R/N224Y/I331L | N224Y/I331L | |
| 147/148 | K15Y/M95V/V149R/G156T/V177P/ | K15Y/V149R/G156T/Y186D/ | + |
| | Y186D/N224Y/K226I/L317A/I331R | N224Y/K226I/L317A/I331R | |
| 133/134 | K15M/G33L/M95V/V149T/A154C/ | K15M/G33L/V149T/A154C/ | + |
| | V177P/Y186R/D195G | Y186R/D195G | |
| 173/174 | K15Y/G33L/K86G/M95V/V149R/ | K15Y/G33L/K86G/V149R/ | + |
| | V177P/D195G/N224Y/I331W | D195G/N224Y/I331W | |
| 161/162 | K15Y/G33L/M95V/V149T/A154C/ | K15Y/G33L/V149T/A154C/ | + |
| | G156T/V177P/Y186D/N224Y/I331L | G156T/Y186D/N224Y/I331L | |
| 143/144 | K15M/G33L/M95V/V149T/A154C/ | K15M/G33L/V149T/A154C/ | + |
| | V177P/D195G/L317A | D195G/L317A | |
| 177/178 | M95V/V149R/V177P/Y186R/D195G/ | V149R/Y186R/D195G/I331W | + |
| | I331W | | |
| 155/156 | K86G/M95V/V149T/A154C/G156T/ | K86G/V149T/A154C/G156T/ | + |
| | V177P/Y186R/D195G/I331L | Y186R/D195G/I331L | |
| 199/200 | M95V/A154C/G156T/V177P/D195G/ | A154C/G156T/D195G/N224Y/ | + |
| | N224Y/K226I | K226I | |
| 211/212 | K15M/M95V/V149R/A154C/V177P/ | K15M/V149R/A154C/D195G/ | + |
| | D195G/I331L | I331L | |
| 195/196 | K15P/G33L/M95V/V149T/G156T/ | K15P/G33L/V149T/G156T/ | + |
| | V177P/D195G/I331W | D195G/I331W | |
| 205/206 | K15P/G33L/M95V/V149R/V177P/ | K15P/G33L/V149R/D195G/ | + |
| | D195G/L317A/I331L | L317A/I331L | |
| 207/208 | K15E/M95V/V149T/A154C/V177P/ | K15E/V149T/A154C/N224Y/ | + |
| | N224Y/I331R | I331R | |
| 179/180 | K15E/M95V/A154C/G156T/V177P/ | K15E/A154C/G156T/Y186D/ | + |
| | Y186D/K226I/I331R | K226I/I331R | |
| 175/176 | K15E/K86G/M95V/V149R/A154C/ | K15E/K86G/V149R/A154C/ | + |
| | G156T/V177P/Y186R/D195G/I331W | G156T/Y186R/D195G/I331W | |

Levels of increased activity were determined relative to the reference polypeptide of SEQ ID NO: 128 and defined as follows: “+” 5.65 to 10.38 (first 50%), “++” > 10.38 (next 30%), “+++” > 13.42 (top 20%)

### Example 10

### Improvements Over SEQ ID NO: 204 in RNA Ligase 2 Activity

SEQ ID NO: 204 was selected as the parent enzyme after screening variants as described in Examples 7-9 above. Libraries of engineered genes were produced using established techniques (e.g., saturation mutagenesis, recombination of previously identified beneficial mutations). The polypeptides encoded by each gene were produced in HIP as described in Example 1 and the cell paste was generated as described in Example 2.

To prepare cells for lysis, 300 μl of 0.1 mg/mL lysozyme in 50 mM Tris-HCl buffer at pH 7.5 was added to the cell paste of each sample. After thorough resuspension, the cells were incubated at 48° C. for 1 hour in a PCR thermocycler. The samples were then centrifuged for 10 min at 4000 rpm and 4° C. and the clear supernatants used in subsequent biocatalytic reactions.

To screen, 20%(v/v) lysate was transferred into reactions containing 50 mM Tris-HCl buffer at pH 7.5, 2 mM MgCl₂, 1 mM DTT, 0.4 mM ATP, and 0.4 mM of an equimolar mixture of the fragments compromising Reaction 2 (sense strands 1-3, and antisense strands 1-2). The reactions were mixed before being incubated for 16 hours at 37° C. in a PCR thermocycler. Afterwards, EDTA was added to each reaction to a final concentration of 20.0 mM and the samples analyzed via UPLC to quantify the remaining fragment concentrations and product yields.

Using peak area from Analytical method 13.1, the % conversion for each sample was calculated as shown here:

$$\% conversion = \frac{area under the curve for all products}{\begin{matrix}Total area under the curve for all \\ products and remaining fragments\end{matrix}} * 100$$

Activity relative to SEQ ID NO: 204 (FIOP) was calculated as the % conversion formed by the variant over % conversion of SEQ ID NO: 204 and shown in Table 10.1.

TABLE 10.1
Relative to SEQ ID NO: 204

| SEQ | Amino Acid | Amino Acid | FIOP |
|---|---|---|---|
| ID | Differences | Differences | Relative to |
| NO: | (Relative to | (Relative to | SEQ ID |
| (nt/aa) | SEQ ID NO: 14) | SEQ ID NO: 204) | NO: 204 |
| 479/480 | H2E/K15Y/H39S/T69D/M95V/Y144W/ | H2E/H39S/T69D/Y144W/ | +++ |
| | V149R/A154C/G156L/V177P/Y186R/ | T156L/M316L/T330M | |
| | D195G/N224Y/K226I/M316L/T330M | | |
| 481/482 | H2V/K15Y/H39S/T69D/M95V/Q119T/ | H2V/H39S/T69D/Q119T/ | +++ |
| | V149R/A154C/G156T/V177P/N185K/ | N185K/M316L | |
| | Y186R/D195G/N224Y/K226I/M316L | | |
| 483/484 | K15Y/T69D/M95V/V149R/A154C/ | T69D/T212I/K256A/T330M | +++ |
| | G156T/V177P/Y186R/D195G/T212I/ | | |
| | N224Y/K226I/K256A/T330M | | |
| 485/486 | K15Y/H39S/M95V/Q119T/V149R/ | H39S/Q119T/K256A/M316L/ | +++ |
| | A154C/G156T/V177P/Y186R/D195G/ | K326R/T330M | |
| | N224Y/K226I/K256A/M316L/K326R/ | | |
| | T330M | | |
| 487/488 | K15Y/T69D/M95V/G116N/Q119T/ | T69D/G116N/Q119T/N185K/ | +++ |
| | V149R/A154C/G156T/V177P/N185K/ | T330M | |
| | Y186R/D195G/N224Y/K226I/T330M | | |
| 489/490 | H2E/K15Y/H39S/M95V/Q119T/Y144W/ | H2E/H39S/Q119T/Y144W/ | +++ |
| | V149R/A154C/G156N/L159F/V177P/ | T156N/L159F/H202Y/K256A/ | |
| | Y186R/D195G/H202Y/N224Y/K226I/ | M316L/K326R/T330M | |
| | K256A/M316L/K326R/T330M | | |
| 491/492 | K15Y/H81Y/M95V/V149R/A154C/ | H81Y/I183V/N185G/N230D | +++ |
| | G156T/V177P/I183V/N185G/Y186R/ | | |
| | D195G/N224Y/K226I/N230D | | |
| 493/494 | H2V/K15Y/H39S/T69D/M95V/G116N/ | H2V/H39S/T69D/G116N/ | +++ |
| | Y144W/V149R/A154C/G156L/V177P/ | Y144W/T156L/N185K | |
| | N185K/Y186R/D195G/N224Y/K226I | | |
| 495/496 | H2E/A14K/K15Y/H39S/T69D/M95V/ | H2E/A14K/H39S/T69D/ | +++ |
| | G116N/Q119T/V149R/A154C/G156T/ | G116N/Q119T/L159F/N185K/ | |
| | L159F/V177P/N185K/Y186R/D195G/ | K256A/K326R | |
| | N224Y/K226I/K256A/K326R | | |
| 497/498 | H2E/K15Y/M95V/G116N/Q119T/V149R/ | H2E/G116N/Q119T/M316L | +++ |
| | A154C/G156T/V177P/Y186R/D195G/ | | |
| | N224Y/K226I/M316L | | |
| 499/500 | K15Y/M95V/G116N/Q119T/Y144W/V149R/ | G116N/Q119T/Y144W/N185K/ | ++ |
| | A154C/G156T/V177P/N185K/Y186R/ | H314W/M316L | |
| | D195G/N224Y/K226I/H314W/M316L | | |
| 501/502 | K15Y/H39S/T69D/M95V/G116N/Y144W/ | H39S/T69D/G116N/Y144W/ | ++ |
| | V149R/A154C/G156T/V177P/K181D/ | K181D/N185K/K256A/T330M | |
| | N185K/Y186R/D195G/N224Y/K226I/ | | |
| | K256A/T330M | | |
| 503/504 | H2E/K15Y/H39S/T69D/M95V/Q119T/ | H2E/H39S/T69D/Q119T/ | ++ |
| | Y144W/V149R/A154C/G156L/V177P/ | Y144W/T156L/N185K/H314W | |
| | N185K/Y186R/D195G/N224Y/K226I/ | | |
| | H314W | | |
| 505/506 | A14K/K15Y/H39S/T69D/M95V/Y144W/ | A14K/H39S/T69D/Y144W/ | ++ |
| | V149R/A154C/G156N/V177P/Y186R/ | T156N/T212I/K256A/H314W/ | |
| | D195G/T212I/N224Y/K226I/K256A/ | M316L/K326R/T330M | |
| | H314W/M316L/K326R/T330M | | |
| 507/508 | K15Y/H39S/T69D/M95V/Y144W/V149R/ | H39S/T69D/Y144W/T156L/ | ++ |
| | A154C/G156L/V177P/N185K/Y186R/ | N185K/M316L/T330M | |
| | D195G/N224Y/K226I/M316L/T330M | | |
| 509/510 | K15Y/M95V/V149R/A154C/G156Q/V177P/ | T156Q/N185G/Q334R/N335H | ++ |
| | N185G/Y186R/D195G/N224Y/K226I/ | | |
| | Q334R/N335H | | |
| 511/512 | K15Y/H39Q/I63V/M95V/V149R/A154C/ | H39Q/I63V/T156Q/N230D/ | ++ |
| | G156Q/V177P/Y186R/D195G/N224Y/ | V257L/T275N/T330R/N335H | |
| | K226I/N230D/V257L/T275N/T330R/ | | |
| | N335H | | |
| 513/514 | K15Y/M95V/V149R/A154C/G156Q/ | T156Q/V266L/M316L/T330R/ | ++ |
| | V177P/Y186R/D195G/N224Y/K226I/V266L/ | Q334R/N335H | |
| | M316L/T330R/Q334R/N335H | | |
| 515/516 | K15Y/H39Q/I63V/M95V/V149R/A154C/ | H39Q/I63V/T156Q/I183V/ | ++ |
| | G156Q/V177P/I183V/N185G/Y186R/ | N185G/N230D/T330R/Q334R | |
| | D195G/N224Y/K226I/N230D/T330R/ | | |
| | Q334R | | |
| 517/518 | K15Y/T69D/M95V/Y144W/V149R/ | T69D/Y144W/M316L | ++ |
| | A154C/G156T/V177P/Y186R/D195G/ | | |
| | N224Y/K226I/M316L | | |
| 519/520 | K15Y/M95V/G116N/Q119T/V149R/ | G116N/Q119T/T156L/H202Y/ | ++ |
| | A154C/G156L/V177P/Y186R/D195G/ | T212I/K326R/T330M | |
| | H202Y/T212I/N224Y/K226I/K326R/T330M | | |
| 521/522 | K15Y/H39S/T69D/M95V/Q119T/S138R/ | H39S/T69D/Q119T/S138R/ | ++ |
| | V149R/A154C/G156T/V177P/K181D/ | K181D/N185K/M316L/K326R | |
| | N185K/Y186R/D195G/N224Y/K226I/ | | |
| | M316L/K326R | | |
| 523/524 | K15Y/H39Q/H81Y/M95V/V149R/ | H39Q/H81Y/T156Q/N230D | ++ |
| | A154C/G156Q/V177P/Y186R/D195G/ | | |
| | N224Y/K226I/N230D | | |
| 525/526 | H2V/A14K/K15Y/T69D/M95V/G116N/ | H2V/A14K/T69D/G116N/ | ++ |
| | Q119T/Y144W/V149R/A154C/G156T/ | Q119T/Y144W/L159F/K326R/ | |
| | L159F/V177P/Y186R/D195G/N224Y/ | T330M | |
| | K226I/K326R/T330M | | |
| 527/528 | K15Y/H39Q/M95V/V149R/A154C/ | H39Q/T156A/Q334R/L345V | ++ |
| | G156A/V177P/Y186R/D195G/N224Y/ | | |
| | K226I/Q334R/L345V | | |
| 529/530 | K15Y/M95V/V149R/A154C/G156T/ | N230D/V266L/Q334R | ++ |
| | V177P/Y186R/D195G/N224Y/K226I/ | | |
| | N230D/V266L/Q334R | | |
| 531/532 | H2V/K15Y/M95V/G116N/Y144W/ | H2V/G116N/Y144W/T156L/ | + |
| | V149R/A154C/G156L/L159F/V177P/ | L159F | |
| | Y186R/D195G/N224Y/K226I | | |
| 533/534 | K15Y/M95V/V149R/A154C/G156A/V177P/ | T156A/I183V/N185G/N230D | + |
| | I183V/N185G/Y186R/D195G/N224Y/ | | |
| | K226I/N230D | | |
| 535/536 | H2E/A14K/K15Y/M95V/G116N/V149R/ | H2E/A14K/G116N/M316L/ | + |
| | A154C/G156T/V177P/Y186R/D195G/ | K326R/T330M | |
| | N224Y/K226I/M316L/K326R/T330M | | |
| 537/538 | K15Y/H39Q/I63V/M95V/V149R/A154C/ | H39Q/I63V/T156A/N230D/ | + |
| | G156A/V177P/Y186R/D195G/N224Y/ | M316L/L345V | |
| | K226I/N230D/M316L/L345V | | |
| 539/540 | K15Y/T69D/M95V/S138R/Y144W/V149R/ | T69D/S138R/Y144W/L159F/ | + |
| | A154C/G156T/L159F/V177P/N185K/ | N185K/H202Y/M316L | |
| | Y186R/D195G/H202Y/N224Y/K226I/ | | |
| | M316L | | |
| 541/542 | K15Y/H39Q/M95V/V149R/A154C/G156Q/ | H39Q/T156Q/V257L/V266L/ | + |
| | V177P/Y186R/D195G/N224Y/K226I/ | M316L/Q334R | |
| | V257L/V266L/M316L/Q334R | | |
| 543/544 | K15Y/M95V/V149R/A154C/G156Q/ | T156Q/V257L/T275N/Q334R | + |
| | V177P/Y186R/D195G/N224Y/K226I/ | | |
| | V257L/T275N/Q334R | | |
| 545/546 | K15Y/M95V/V149R/A154C/G156T/ | I183V | + |
| | V177P/I183V/Y186R/D195G/N224Y/K226I | | |
| 547/548 | K15Y/K89W/M95V/V149R/A154C/G156A/ | K89W/T156A/N185G/N230D/ | + |
| | V177P/N185G/Y186R/D195G/N224Y/ | M316L/L345V | |
| | K226I/N230D/M316L/L345V | | |
| 549/550 | H2E/A14K/K15Y/M95V/Q119T/V149R/ | H2E/A14K/Q119T | + |
| | A154C/G156T/V177P/Y186R/D195G/ | | |
| | N224Y/K226I | | |
| 551/552 | K15Y/M95V/Y144W/V149R/A154C/G156L/ | Y144W/T156L/M316L/T330M | + |
| | V177P/Y186R/D195G/N224Y/K226I/ | | |
| | M316L/T330M | | |
| 553/554 | K15Y/H39Q/H81Y/M95V/V149R/A154C/ | H39Q/H81Y/N185G/M316L/ | + |
| | G156T/V177P/N185G/Y186R/D195G/ | Q334R | |
| | N224Y/K226I/M316L/Q334R | | |
| 555/556 | K15Y/K89H/M95V/V149R/A154C/G156T/ | K89H/N230D/V266L/L345V | + |
| | V177P/Y186R/D195G/N224Y/K226I/ | | |
| | N230D/V266L/L345V | | |
| 557/558 | K15Y/H39Q/M95V/V149R/A154C/G156Q/ | H39Q/T156Q/N230D/T275N/ | + |
| | V177P/Y186R/D195G/N224Y/K226I/ | M316L | |
| | N230D/T275N/M316L | | |
| 559/560 | K15Y/H39Q/M95V/V149R/A154C/G156Q/ | H39Q/T156Q/N230D/V266L/ | + |
| | V177P/Y186R/D195G/N224Y/K226I/ | Q334R/N335H | |
| | N230D/V266L/Q334R/N335H | | |
| 561/562 | K15Y/M95V/V149R/A154C/G156Q/V177P/ | T156Q/N185G/T275N/M316L/ | + |
| | N185G/Y186R/D195G/N224Y/K226I/ | T330R | |
| | T275N/M316L/T330R | | |
| 563/564 | K15Y/M95V/Q119T/V149R/A154C/G156N/ | Q119T/T156N/H202Y | + |
| | V177P/Y186R/D195G/H202Y/N224Y/ | | |
| | K226I | | |
| 565/566 | K15Y/M95V/V149R/A154C/G156T/V177P/ | N230D/V257L/T275N/M316L/ | + |
| | Y186R/D195G/N224Y/K226I/N230D/ | T330R/L345V | |
| | V257L/T275N/M316L/T330R/L345V | | |
| 567/568 | K15Y/M95V/V149R/A154C/G156T/ | N230D | + |
| | V177P/Y186R/D195G/N224Y/K226I/N230D | | |
| 569/570 | K15Y/H81Y/M95V/V149R/A154C/G156Q/ | H81Y/T156Q/T330R/N335H/ | + |
| | V177P/Y186R/D195G/N224Y/K226I/ | L345V | |
| | T330R/N335H/L345V | | |
| 571/572 | K15Y/M95V/V149R/A154C/G156A/ | T156A/I183V/N230D/V266L | + |
| | V177P/I183V/Y186R/D195G/N224Y/K226I/ | | |
| | N230D/V266L | | |
| 573/574 | K15Y/H39S/M95V/V149R/A154C/G156T/ | H39S/N185K | + |
| | V177P/N185K/Y186R/D195G/N224Y/ | | |
| | K226I | | |
| 575/576 | K15Y/H39Q/H81Y/M95V/V149R/A154C/ | H39Q/H81Y/I183V/T275N/ | + |
| | G156T/V177P/I183V/Y186R/D195G/ | M316L/Q334R/L345V | |
| | N224Y/K226I/T275N/M316L/Q334R/ | | |
| | L345V | | |
| 577/578 | K15Y/M95V/V149R/A154C/G156A/V177P/ | T156A/N230D/V257L/M316L | + |
| | Y186R/D195G/N224Y/K226I/N230D/ | | |
| | V257L/M316L | | |
| 579/580 | K15Y/H39S/M95V/G116N/V149R/A154C/ | H39S/G116N/T156N | + |
| | G156N/V177P/Y186R/D195G/N224Y/ | | |
| | K226I | | |
| 581/582 | K15Y/M95V/V149R/A154C/G156T/V177P/ | I183V/N185G/V257L/T275N/ | + |
| | I183V/N185G/Y186R/D195G/N224Y/ | M316L/T330R/Q334R | |
| | K226I/V257L/T275N/M316L/T330R/ | | |
| | Q334R | | |

Levels of increased activity were determined relative to the reference polypeptide of SEQ ID NO: 204 and defined as follows: “+” 1.75 to 2.02 (first 50%), “++” > 2.02 (next 30%), “+++” > 2.48 (top 20%)

### Example 11

### Improvements Over SEQ ID NO: 204 in RNA Ligase 2 Activity

SEQ ID NO: 204 was selected as the parent enzyme after screening variants as described in Examples 7-9 above, lop variants were selected from Example 10 above to test again under another condition.

Libraries of engineered genes were produced using established techniques (e.g., saturation mutagenesis, recombination of previously identified beneficial mutations). The polypeptides encoded by each gene were produced in HIP as described in Example 1 and the cell paste was generated as described in Example 2.

To prepare cells for lysis, 300 μl of 0.1 mg/mL lysozyme in 50 mM Tris-HCl buffer at pH 7.5 was added to the cell paste of each sample. After thorough resuspension, the cells were incubated at 48° C. for 1 hr in a PCR thermocycler. The samples were then centrifuged for 10 min at 4000 rpm and 4° C. and the clear supernatants used in subsequent biocatalytic reactions.

To screen, 20%(v/v) lysate was transferred into reactions containing 50 mM Tris-HCl buffer at pH 7.5, 2 mM MgCl₂, 1 mM DTT, 0.4 mM ATP, and 3 mM of an equimolar mixture of the fragments compromising Reaction 2 (sense strands 1-3, and antisense strands 1-2). The reactions were mixed before being incubated for 16 hours at 37° C. in a PCR thermocycler. Afterwards, EDTA was added to each reaction to a final concentration of 20 mM and the samples analyzed via UPLC to quantify the remaining fragment concentrations and product yields.

Using peak area from Analytical method 13. 1, the % conversion for each sample was calculated as shown here:

$$\% conversion = \frac{area under the curve for all products}{\begin{matrix}Total area under the curve for all \\ products and remaining fragments\end{matrix}} * 100$$

Activity relative to SEQ ID NO: 204 (FIOP) was calculated as the % conversion formed by the variant over % conversion of SEQ ID NO: 204 and shown in Table 11.1.

TABLE 11.1
Relative to SEQ ID NO: 204

| SEQ | Amino Acid | Amino Acid | FIOP |
|---|---|---|---|
| ID | Differences | Differences | Relative to |
| NO: | (Relative to | (Relative to | SEQ ID |
| (nt/aa) | SEQ ID NO: 14) | SEQ ID NO: 204) | NO: 204 |
| 479/480 | H2E/K15Y/H39S/T69D/M95V/Y144W/ | H2E/H39S/T69D/Y144W/ | +++ |
| | V149R/A154C/G156L/V177P/Y186R/ | T156L/M316L/T330M | |
| | D195G/N224Y/K226I/M316L/T330M | | |
| 483/484 | K15Y/T69D/M95V/V149R/A154C/G156T/ | T69D/T212I/K256A/T330M | +++ |
| | V177P/Y186R/D195G/T212I/N224Y/ | | |
| | K226I/K256A/T330M | | |
| 481/482 | H2V/K15Y/H39S/T69D/M95V/Q119T/ | H2V/H39S/T69D/Q119T/ | +++ |
| | V149R/A154C/G156T/V177P/N185K/ | N185K/M316L | |
| | Y186R/D195G/N224Y/K226I/M316L | | |
| 485/486 | K15Y/H39S/M95V/Q119T/V149R/ | H39S/Q119T/K256A/ | +++ |
| | A154C/G156T/V177P/Y186R/D195G/N224Y/ | M316L/K326R/T330M | |
| | K226I/K256A/M316L/K326R/T330M | | |
| 489/490 | H2E/K15Y/H39S/M95V/Q119T/Y144W/ | H2E/H39S/Q119T/Y144W/ | +++ |
| | V149R/A154C/G156N/L159F/V177P/ | T156N/L159F/H202Y/K256 | |
| | Y186R/D195G/H202Y/N224Y/K226I/K256A/ | A/M316L/K326R/T330M | |
| | M316L/K326R/T330M | | |
| 513/514 | K15Y/M95V/V149R/A154C/G156Q/ | T156Q/V266L/M316L/ | +++ |
| | 7V17P/Y186R/D195G/N224Y/K226I/V266L/ | T330R/Q334R/N335H | |
| | M316L/T330R/Q334R/N335H | | |
| 487/488 | K15Y/T69D/M95V/G116N/Q119T/V149 | T69D/G116N/Q119T/ | +++ |
| | R/A154C/G156T/V177P/N185K/Y186R/ | N185K/T330M | |
| | D195G/N224Y/K226I/T330M | | |
| 501/502 | K15Y/H39S/T69D/M95V/G116N/Y144W/ | H39S/T69D/G116N/Y144W/ | +++ |
| | V149R/A154C/G156T/V177P/K181D/ | K181D/N185K/K256A/ | |
| | N185K/Y186R/D195G/N224Y/K226I/ | T330M | |
| | K256A/T330M | | |
| 527/528 | K15Y/H39Q/M95V/V149R/A154C/G156A/ | H39Q/T156A/Q334R/ | +++ |
| | V177P/Y186R/D195G/N224Y/K226I/ | L345V | |
| | Q334R/L345V | | |
| 529/530 | K15Y/M95V/V149R/A154C/G156T/V177P/ | N230D/V266L/Q334R | ++ |
| | Y186R/D195G/N224Y/K226I/N230D/ | | |
| | V266L/Q334R | | |
| 515/516 | K15Y/H39Q/I63V/M95V/V149R/A154C/ | H39Q/I63V/T156Q/I183V/ | ++ |
| | G156Q/V177P/I183V/N185G/Y186R/D195G/ | N185G/N230D/T330R/Q334R | |
| | N224Y/K226I/N230D/T330R/Q334R | | |
| 547/548 | K15Y/K89W/M95V/V149R/A154C/G156A/ | K89W/T156A/N185G/N230D/ | ++ |
| | V177P/N185G/Y186R/D195G/N224Y/ | M316L/L345V | |
| | K226I/N230D/M316L/L345V | | |
| 559/560 | K15Y/H39Q/M95V/V149R/A154C/G156Q/ | H39Q/T156Q/N230D/ | ++ |
| | V177P/Y186R/D195G/N224Y/K226I/ | V266L/Q334R/N335H | |
| | N230D/V266L/Q334R/N335H | | |
| 511/512 | K15Y/H39Q/I63V/M95V/V149R/A154C/ | H39Q/I63V/T156Q/N230D/ | ++ |
| | G156Q/V177P/Y186R/D195G/N224Y/ | V257L/T275N/T330R/ | |
| | K226I/N230D/V257L/T275N/T330R/N335H | N335H | |
| 555/556 | K15Y/K89H/M95V/V149R/A154C/G156T/ | K89H/N230D/V266L/ | ++ |
| | V177P/Y186R/D195G/N224Y/K226I/ | L345V | |
| | N230D/V266L/L345V | | |
| 569/570 | K15Y/H81Y/M95V/V149R/A154C/G156Q/ | H81Y/T156Q/T330R/ | ++ |
| | V177P/Y186R/D195G/N224Y/K226I/ | N335H/L345V | |
| | T330R/N335H/L345V | | |
| 543/544 | K15Y/M95V/V149R/A154C/G156Q/ | T156Q/V257L/T275N/ | ++ |
| | V177P/Y186R/D195G/N224Y/K226I/V257L/ | Q334R | |
| | T275N/Q334R | | |
| 571/572 | K15Y/M95V/V149R/A154C/G156A/ | T156A/I183V/N230D/ | ++ |
| | V177P/I183V/Y186R/D195G/N224Y/K226I/ | V266L | |
| | N230D/V266L | | |
| 507/508 | K15Y/H39S/T69D/M95V/Y144W/V149R/ | H39S/T69D/Y144W/T156L/ | ++ |
| | A154C/G156L/V177P/N185K/Y186R/ | N185K/M316L/T330M | |
| | D195G/N224Y/K226I/M316L/T330M | | |
| 517/518 | K15Y/T69D/M95V/Y144W/V149R/ | T69D/Y144W/M316L | ++ |
| | A154C/G156T/V177P/Y186R/D195G/N224Y/ | | |
| | K226I/M316L | | |
| 493/494 | H2V/K15Y/H39S/T69D/M95V/G116N/ | H2V/H39S/T69D/G116N/ | ++ |
| | Y144W/V149R/A154C/G156L/V177P/ | Y144W/T156L/N185K | |
| | N185K/Y186R/D195G/N224Y/K226I | | |
| 533/534 | K15Y/M95V/V149R/A154C/G156A/ | T156A/I183V/N185G/ | ++ |
| | V177P/I183V/N185G/Y186R/D195G/N224Y/ | N230D | |
| | K226I/N230D | | |
| 541/542 | K15Y/H39Q/M95V/V149R/A154C/G156Q/ | H39Q/T156Q/V257L/ | ++ |
| | V177P/Y186R/D195G/N224Y/K226I/ | V266L/M316L/Q334R | |
| | V257L/V266L/M316L/Q334R | | |
| 567/568 | K15Y/M95V/V149R/A154C/G156T/ | N230D | + |
| | V177P/Y186R/D195G/N224Y/K226I/N230D | | |
| 565/566 | K15Y/M95V/V149R/A154C/G156T/V177P/ | N230D/V257L/T275N/ | + |
| | Y186R/D195G/N224Y/K226I/N230D/ | M316L/T330R/L345V | |
| | V257L/T275N/M316L/T330R/L345V | | |
| 539/540 | K15Y/T69D/M95V/S138R/Y144W/V149R/ | T69D/S138R/Y144W/L159F/ | + |
| | A154C/G156T/L159F/V177P/N185K/ | N185K/H202Y/M316L | |
| | Y186R/D195G/H202Y/N224Y/K226I/ | | |
| | M316L | | |
| 497/498 | H2E/K15Y/M95V/G116N/Q119T/V149R/ | H2E/G116N/Q119T/M316L | + |
| | A154C/G156T/V177P/Y186R/D195G/ | | |
| | N224Y/K226I/M316L | | |
| 553/554 | K15Y/H39Q/H81Y/M95V/V149R/A154C/ | H39Q/H81Y/N185G/M316L/ | + |
| | G156T/V177P/N185G/Y186R/D195G/ | Q334R | |
| | N224Y/K226I/M316L/Q334R | | |
| 503/504 | H2E/K15Y/H39S/T69D/M95V/Q119T/ | H2E/H39S/T69D/Q119T/ | + |
| | 4Y14W/V149R/A154C/G156L/V177P/ | Y144W/T156L/N185K/ | |
| | N185K/Y186R/D195G/N224Y/K226I/H314W | H314W | |
| 563/564 | K15Y/M95V/Q119T/V149R/A154C/ | Q119T/T156N/H202Y | + |
| | G156N/V177P/Y186R/D195G/H202Y/N224Y/ | | |
| | K226I | | |
| 561/562 | K15Y/M95V/V149R/A154C/G156Q/ | T156Q/N185G/T275N/ | + |
| | V177P/N185G/Y186R/D195G/N224Y/ | M316L/T330R | |
| | K226I/T275N/M316L/T330R | | |
| 557/558 | K15Y/H39Q/M95V/V149R/A154C/G156Q/ | H39Q/T156Q/N230D/ | + |
| | V177P/Y186R/D195G/N224Y/K226I/ | T275N/M316L | |
| | N230D/T275N/M316L | | |
| 523/524 | K15Y/H39Q/H81Y/M95V/V149R/A154C/ | H39Q/H81Y/T156Q/N230D | + |
| | G156Q/V177P/Y186R/D195G/N224Y/ | | |
| | K226I/N230D | | |
| 491/492 | K15Y/H81Y/M95V/V149R/A154C/G156T/ | H81Y/I183V/N185G/N230D | + |
| | V177P/I183V/N185G/Y186R/D195G/ | | |
| | N224Y/K226I/N230D | | |
| 531/532 | H2V/K15Y/M95V/G116N/Y144W/V149R/ | H2V/G116N/Y144W/ | + |
| | A154C/G156L/L159F/V177P/Y186R/ | T156L/L159F | |
| | D195G/N224Y/K226I | | |
| 505/506 | A14K/K15Y/H39S/T69D/M95V/Y144W/ | A14K/H39S/T69D/Y144W/ | + |
| | V149R/A154C/G156N/V177P/Y186R/ | T156N/T212I/K256A/H314W/ | |
| | D195G/T212I/N224Y/K226I/K256A/ | M316L/K326R/T330M | |
| | H314W/M316L/K326R/T330M | | |
| 495/496 | H2E/A14K/K15Y/H39S/T69D/M95V/ | H2E/A14K/H39S/T69D/G116N/ | + |
| | G116N/Q119T/V149R/A154C/G156T/ | Q119T/L159F/N185K/K | |
| | L159F/V177P/N185K/Y186R/D195G/ | 256A/K326R | |
| | N224Y/K226I/K256A/K326R | | |
| 521/522 | K15Y/H39S/T69D/M95V/Q119T/S138R/ | H39S/T69D/Q119T/S138R/ | + |
| | V149R/A154C/G156T/V177P/K181D/ | K181D/N185K/M316L/ | |
| | N185K/Y186R/D195G/N224Y/K226I/ | K326R | |
| | M316L/K326R | | |
| 509/510 | K15Y/M95V/V149R/A154C/G156Q/ | T156Q/N185G/Q334R/ | + |
| | V177P/N185G/Y186R/D195G/N224Y/K226I/ | N335H | |
| | Q334R/N335H | | |
| 525/526 | H2V/A14K/K15Y/T69D/M95V/G116N/ | H2V/A14K/T69D/G116N/ | + |
| | Q119T/Y144W/V149R/A154C/G156T/ | Q119T/Y144W/L159F/K326R/ | |
| | L159F/V177P/Y186R/D195G/N224Y/ | T330M | |
| | K226I/K326R/T330M | | |
| 537/538 | K15Y/H39Q/I63V/M95V/V149R/A154C/ | H39Q/I63V/T156A/N230D/ | + |
| | G156A/V177P/Y186R/D195G/N224Y/ | M316L/L345V | |
| | K226I/N230D/M316L/L345V | | |
| 519/520 | K15Y/M95V/G116N/Q119T/V149R/ | G116N/Q119T/T156L/ | + |
| | A154C/G156L/V177P/Y186R/D195G/H202Y/ | H202Y/T212I/K326R/T330M | |
| | T212I/N224Y/K226I/K326R/T330M | | |
| 499/500 | K15Y/M95V/G116N/Q119T/Y144W/ | G116N/Q119T/Y144W/ | + |
| | V149R/A154C/G156T/V177P/N185K/Y186R/ | N185K/H314W/M316L | |
| | D195G/N224Y/K226I/H314W/M316L | | |
| 545/546 | K15Y/M95V/V149R/A154C/G156T/ | I183V | + |
| | V177P/I183V/Y186R/D195G/N224Y/K226I | | |
| 551/552 | K15Y/M95V/Y144W/V149R/A154C/ | Y144W/T156L/M316L/ | + |
| | G156L/V177P/Y186R/D195G/N224Y/ | T330M | |
| | K226I/M316L/T330M | | |
| 535/536 | H2E/A14K/K15Y/M95V/G116N/V149R/ | H2E/A14K/G116N/M316L/ | + |
| | A154C/G156T/V177P/Y186R/D195G/ | K326R/T330M | |
| | N224Y/K226I/M316L/K326R/T330M | | |
| 549/550 | H2E/A14K/K15Y/M95V/Q119T/V149R/ | H2E/A14K/Q119T | + |
| | A154C/G156T/V177P/Y186R/D195G/ | | |
| | N224Y/K226I | | |

Levels of increased activity were determined relative to the reference polypeptide of SEQ ID NO: 204 and defined as follows: “+” 1.45 to 2.33 (first 50%), “++” > 2.33 (next 30%), “+++” > 2.50 (top 20%)

### Example 12

### Improvements Over SEQ ID NO: 204 in RNA Ligase 2 Activity

The same samples from Example I11 were tested again, but with a higher fragment load. To screen, 20%(v/v) lysate was transferred into reactions containing 50 mM Tris-HCl buffer at pH 7.5, 2 mM MgCl₂, 1 mM DTT, 0.4 mM ATP, and 4 mM of an equimolar mixture of the fragments compromising Reaction 2 (sense strands 1-3, and antisense strands 1-2). The reactions were mixed before being incubated for 16 hr at 37° C. in a PCR thermocycler. Afterwards, EDTA was added to each reaction to a final concentration of 20 mM and the samples analyzed via UPLC to quantify the remaining fragment concentrations and product yields.

Using peak area from Analytical method 13.1, the % conversion for each sample was calculated as shown here:

$$\% conversion = \frac{area under the curve for all products}{\begin{matrix}Total area under the curve for all \\ products and remaining fragments\end{matrix}} * 100$$

Activity relative to SEQ ID NO: 204 (FIOP) was calculated as the % conversion formed by the variant over % conversion of SEQ ID NO: 204 and shown in Table 12.1.

TABLE 12.1
Relative to SEQ ID NO: 204

| SEQ | Amino Acid | Amino Acid | FIOP |
|---|---|---|---|
| ID | Differences | Differences | Relative to |
| NO: | (Relative to | (Relative to | SEQ ID |
| (nt/aa) | SEQ ID NO: 14) | SEQ ID NO: 204) | NO: 204 |
| 479/480 | H2E/K15Y/H39S/T69D/M95V/Y144W/ | H2E/H39S/T69D/Y144W/ | +++ |
| | V149R/A154C/G156L/V177P/Y186R/ | T156L/M316L/T330M | |
| | D195G/N224Y/K226I/M316L/T330M | | |
| 501/502 | K15Y/T69D/M95V/V149R/A154C/ | H39S/T69D/G116N/Y144W/ | +++ |
| | G156T/V177P/Y186R/D195G/T212I/ | K181D/N185K/K256A/T330M | |
| | N224Y/K226I/K256A/T330M | | |
| 481/482 | H2V/K15Y/H39S/T69D/M95V/Q119T/ | H2V/H39S/T69D/Q119T/ | +++ |
| | V149R/A154C/G156T/V177P/N185K/ | N185K/M316L | |
| | Y186R/D195G/N224Y/K226I/M316L | | |
| 483/484 | K15Y/H39S/M95V/Q119T/V149R/ | T69D/T212I/K256A/T330M | +++ |
| | A154C/G156T/V177P/Y186R/D195G/N224Y/ | | |
| | K226I/K256A/M316L/K326R/T330M | | |
| 513/514 | H2E/K15Y/H39S/M95V/Q119T/Y144W/ | T156Q/V266L/M316L/T330R/ | +++ |
| | V149R/A154C/G156N/L159F/V177P/ | Q334R/N335H | |
| | Y186R/D195G/H202Y/N224Y/K226I/ | | |
| | K256A/M316L/K326R/T330M | | |
| 489/490 | K15Y/M95V/V149R/A154C/G156Q/ | H2E/H39S/Q119T/Y144W/T15 | +++ |
| | V177P/Y186R/D195G/N224Y/K226I/V266L/ | 6N/L159F/H202Y/K256A/M31 | |
| | M316L/T330R/Q334R/N335H | 6L/K326R/T330M | |
| 529/530 | K15Y/T69D/M95V/G116N/Q119T/V149R/ | N230D/V266L/Q334R | +++ |
| | A154C/G156T/V177P/N185K/Y186R/ | | |
| | D195G/N224Y/K226I/T330M | | |
| 527/528 | K15Y/H39S/T69D/M95V/G116N/Y144W/ | H39Q/T156A/Q334R/L345V | +++ |
| | V149R/A154C/G156T/V177P/K181D/ | | |
| | N185K/Y186R/D195G/N224Y/K226I/ | | |
| | K256A/T330M | | |
| 515/516 | K15Y/H39Q/M95V/V149R/A154C/G156A/ | H39Q/I63V/T156Q/I183V/ | +++ |
| | V177P/Y186R/D195G/N224Y/K226I/ | N185G/N230D/T330R/Q334R | |
| | Q334R/L345V | | |
| 541/542 | K15Y/M95V/V149R/A154C/G156T/ | H39Q/T156Q/V257L/V266L/ | ++ |
| | V177P/Y186R/D195G/N224Y/K226I/ | M316L/Q334R | |
| | N230D/V266L/Q334R | | |
| 565/566 | K15Y/H39Q/I63V/M95V/V149R/ | N230D/V257L/T275N/M316L/ | ++ |
| | A154C/G156Q/V177P/I183V/N185G/Y186R/ | T330R/L345V | |
| | D195G/N224Y/K226I/N230D/T330R/ | | |
| | Q334R | | |
| 493/494 | K15Y/K89W/M95V/V149R/A154C/ | H2V/H39S/T69D/G116N/ | ++ |
| | G156A/V177P/N185G/Y186R/D195G/N224 | Y144W/T156L/N185K | |
| | Y/K226I/N230D/M316L/L345V | | |
| 571/572 | K15Y/H39Q/M95V/V149R/A154C/ | T156A/I183V/N230D/V266L | ++ |
| | G156Q/V177P/Y186R/D195G/N224Y/ | | |
| | K226I/N230D/V266L/Q334R/N335H | | |
| 547/548 | K15Y/H39Q/I63V/M95V/V149R/A154C/ | K89W/T156A/N185G/N230D/ | ++ |
| | G156Q/V177P/Y186R/D195G/N224Y/ | M316L/L345V | |
| | K226I/N230D/V257L/T275N/T330R/ | | |
| | N335H | | |
| 559/560 | K15Y/K89H/M95V/V149R/A154C/ | H39Q/T156Q/N230D/V266L/ | ++ |
| | G156T/V177P/Y186R/D195G/N224Y/K226I/ | Q334R/N335H | |
| | N230D/V266L/L345V | | |
| 511/512 | K15Y/H81Y/M95V/V149R/A154C/G156Q/ | H39Q/I63V/T156Q/N230D/ | ++ |
| | V177P/Y186R/D195G/N224Y/K226I/ | V257L/T275N/T330R/N335H | |
| | T330R/N335H/L345V | | |
| 555/556 | K15Y/M95V/V149R/A154C/G156Q/ | K89H/N230D/V266L/L345V | ++ |
| | V177P/Y186R/D195G/N224Y/K226I/V257L/ | | |
| | T275N/Q334R | | |
| 533/534 | K15Y/M95V/V149R/A154C/G156A/ | T156A/I183V/N185G/N230D | ++ |
| | V177P/I183V/Y186R/D195G/N224Y/K226I/ | | |
| | N230D/V266L | | |
| 485/486 | K15Y/H39S/T69D/M95V/Y144W/V149R/ | H39S/Q119T/K256A/M316L/ | ++ |
| | A154C/G156L/V177P/N185K/Y186R/ | K326R/T330M | |
| | D195G/N224Y/K226I/M316L/T330M | | |
| 487/488 | K15Y/T69D/M95V/Y144W/V149R/ | T69D/G116N/Q119T/N185K/ | ++ |
| | A154C/G156T/V177P/Y186R/D195G/N224Y/ | T330M | |
| | K226I/M316L | | |
| 567/568 | H2V/K15Y/H39S/T69D/M95V/G116N/ | N230D | ++ |
| | Y144W/V149R/A154C/G156L/V177P/ | | |
| | N185K/Y186R/D195G/N224Y/K226I | | |
| 507/508 | K15Y/M95V/V149R/A154C/G156A/ | H39S/T69D/Y144W/T156L/ | ++ |
| | V177P/I183V/N185G/Y186R/D195G/ | N185K/M316L/T330M | |
| | N224Y/K226I/N230D | | |
| 543/544 | K15Y/H39Q/M95V/V149R/A154C/ | T156Q/V257L/T275N/Q334R | ++ |
| | G156Q/V177P/Y186R/D195G/N224Y/K226I/ | | |
| | V257L/V266L/M316L/Q334R | | |
| 569/570 | K15Y/M95V/V149R/A154C/G156T/ | H81Y/T156Q/T330R/N335H/ | + |
| | V177P/Y186R/D195G/N224Y/K226I/N230D | L345V | |
| 503/504 | K15Y/M95V/V149R/A154C/G156T/ | H2E/H39S/T69D/Q119T/ | + |
| | V177P/Y186R/D195G/N224Y/K226I/N230D/ | Y144W/T156L/N185K/H314W | |
| | V257L/T275N/M316L/T330R/L345V | | |
| 491/492 | K15Y/T69D/M95V/S138R/Y144W/ | H81Y/I183V/N185G/N230D | + |
| | V149R/A154C/G156T/L159F/V177P/N185K/ | | |
| | Y186R/D195G/H202Y/N224Y/K226I/ | | |
| | M316L | | |
| 523/524 | H2E/K15Y/M95V/G116N/Q119T/V149R/ | H39Q/H81Y/T156Q/N230D | + |
| | A154C/G156T/V177P/Y186R/D195G/ | | |
| | N224Y/K226I/M316L | | |
| 557/558 | K15Y/H39Q/H81Y/M95V/V149R/ | H39Q/T156Q/N230D/T275N/ | + |
| | A154C/G156T/V177P/N185G/Y186R/D195G/ | M316L | |
| | N224Y/K226I/M316L/Q334R | | |
| 553/554 | H2E/K15Y/H39S/T69D/M95V/Q119T/ | H39Q/H81Y/N185G/M316L/ | + |
| | Y144W/V149R/A154C/G156L/V177P/ | Q334R | |
| | N185K/Y186R/D195G/N224Y/K226I/ | | |
| | H314W | | |
| 517/518 | K15Y/M95V/Q119T/V149R/A154C/ | T69D/Y144W/M316L | + |
| | G156N/V177P/Y186R/D195G/H202Y/ | | |
| | N224Y/K226I | | |
| 497/498 | K15Y/M95V/V149R/A154C/G156Q/ | H2E/G116N/Q119T/M316L | + |
| | V177P/N185G/Y186R/D195G/N224Y/ | | |
| | K226I/T275N/M316L/T330R | | |
| 539/540 | K15Y/H39Q/M95V/V149R/A154C/ | T69D/S138R/Y144W/L159F/ | + |
| | G156Q/V177P/Y186R/D195G/N224Y/K226I/ | N185K/H202Y/M316L | |
| | N230D/T275N/M316L | | |
| 521/522 | K15Y/H39Q/H81Y/M95V/V149R/ | H39S/T69D/Q119T/S138R/ | + |
| | A154C/G156Q/V177P/Y186R/D195G/ | K181D/N185K/M316L/K326R | |
| | N224Y/K226I/N230D | | |
| 495/496 | K15Y/H81Y/M95V/V149R/A154C/ | H2E/A14K/H39S/T69D/ | + |
| | G156T/V177P/I183V/N185G/Y186R/D195G/ | G116N/Q119T/L159F/N185K/ | |
| | N224Y/K226I/N230D | K256A/K326R | |
| 505/506 | H2V/K15Y/M95V/G116N/Y144W/ | A14K/H39S/T69D/Y144W/ | + |
| | V149R/A154C/G156L/L159F/V177P/Y186R/ | T156N/T212I/K256A/H314W/ | |
| | D195G/N224Y/K226I | M316L/K326R/T330M | |
| 563/564 | A14K/K15Y/H39S/T69D/M95V/Y144W/ | Q119T/T156N/H202Y | + |
| | V149R/A154C/G156N/V177P/Y186R/ | | |
| | D195G/T212I/N224Y/K226I/K256A/ | | |
| | H314W/M316L/K326R/T330M | | |
| 531/532 | H2E/A14K/K15Y/H39S/T69D/M95V/ | H2V/G116N/Y144W/T156L/ | + |
| | G116N/Q119T/V149R/A154C/G156T/ | L159F | |
| | L159F/V177P/N185K/Y186R/D195G/N224Y/ | | |
| | K226I/K256A/K326R | | |
| 537/538 | K15Y/H39S/T69D/M95V/Q119T/S138R/ | H39Q/I63V/T156A/N230D/ | + |
| | V149R/A154C/G156T/V177P/K181D/ | M316L/L345V | |
| | N185K/Y186R/D195G/N224Y/K226I/ | | |
| | M316L/K326R | | |
| 509/510 | K15Y/M95V/V149R/A154C/G156Q/ | T156Q/N185G/Q334R/N335H | + |
| | V177P/N185G/Y186R/D195G/N224Y/ | | |
| | K226I/Q334R/N335H | | |
| 499/500 | H2V/A14K/K15Y/T69D/M95V/G116N/ | G116N/Q119T/Y144W/N185K/ | + |
| | Q119T/Y144W/V149R/A154C/G156T/ | H314W/M316L | |
| | L159F/V177P/Y186R/D195G/N224Y/ | | |
| | K226I/K326R/T330M | | |
| 525/526 | K15Y/H39Q/I63V/M95V/V149R/A154C/ | H2V/A14K/T69D/G116N/ | + |
| | G156A/V177P/Y186R/D195G/N224Y/ | Q119T/Y144W/L159F/K326R/ | |
| | K226I/N230D/M316L/L345V | T330M | |
| 519/520 | K15Y/M95V/G116N/Q119T/V149R/ | G116N/Q119T/T156L/H202Y/ | + |
| | A154C/G156L/V177P/Y186R/D195G/ | T212I/K326R/T330M | |
| | H202Y/T212I/N224Y/K226I/K326R/T330M | | |
| 561/562 | K15Y/M95V/G116N/Q119T/Y144W/ | T156Q/N185G/T275N/M316L/ | + |
| | V149R/A154C/G156T/V177P/N185K/Y186R/ | T330R | |
| | D195G/N224Y/K226I/H314W/M316L | | |
| 545/546 | K15Y/M95V/V149R/A154C/G156T/ | I183V | + |
| | V177P/I183V/Y186R/D195G/N224Y/K226I | | |
| 551/552 | K15Y/M95V/Y144W/V149R/A154C/ | Y144W/T156L/M316L/T330M | + |
| | G156L/V177P/Y186R/D195G/N224Y/ | | |
| | K226I/M316L/T330M | | |
| 535/536 | H2E/A14K/K15Y/M95V/G116N/V149R/ | H2E/A14K/G116N/M316L/ | + |
| | A154C/G156T/V177P/Y186R/D195G/ | K326R/T330M | |
| | N224Y/K226I/M316L/K326R/T330M | | |
| 549/550 | H2E/A14K/K15Y/M95V/Q119T/V149R/ | H2E/A14K/Q119T | + |
| | A154C/G156T/V177P/Y186R/D195G/ | | |
| | N224Y/K226I | | |

Levels of increased activity were determined relative to the reference polypeptide of SEQ ID NO: 204 and defined as follows: “+” 1.36 to 3.03 (first 50%), “++” > 3.03 (next 30%), “+++” > 3.52 (top 20%)

### Example 13

### Analytical Detection of RNA Oligomers by UPLC-UV

Data described in Examples 4-12 was collected using the analytical method provided in Table 13.1. The method provided herein finds use in analyzing the variants produced using the present disclosure.

TABLE 13.1
Analytical Method

| Instrument | Agilent 1290-UPLC | | |
|---|---|---|---|
| Column | 50 × 2.1 mm Waters BEH C18, 1.7 μm particle size (180 Å) | | |
| Mobile Phase | A: 10 mM DIPEA, 100 mM HFIP in water, B: Acetonitrile | | |
| Gradient | Time (min) | % A | % B |
| | 0 | 95 | 5 |
| | 1 | 91.5 | 8.5 |
| | 2 | 91 | 9 |
| | 3 | 86 | 14 |
| | 3.1 | 10 | 90 |
| | 3.8 | 10 | 90 |
| | 3.9 | 95 | 5 |
| | 5.2 | 95 | 5 |

| Flow Rate | 0.4 | mL/min |
|---|---|---|
| Run Time | 5.5 | min |
| Product Elution order | peak name | minutes |
| | sense strand fragment 5 | 0.6 |
| | antisense strand fragment 4 | 1.1 |
| | antisense strand fragment 3 | 1.3 |
| | sense strand fragment 2 | 1.8 |
| | sense strand fragment 3 | 1.9 |
| | antisense strand fragment 2 | 2.1 |
| | antisense strand fragment 1 | 2.4 |
| | sense strand fragment 1 | 2.6 |
| | sense strand fragment 4 | 3 |
| | Product 2 | 3.2 |
| | Product 1 | 3.4 |
| Column Temperature | 75° | C. |
| Injection Volume | 2 | μL |

| | |
|---|---|
| Detection | UV 258 nm (20 Hz) |

While the invention has been described with reference to the specific embodiments, various changes can be made and equivalents can be substituted to adapt to a particular situation, material, composition of matter, process, process step or steps, thereby achieving benefits of the invention without departing from the scope of what is claimed.

For all purposes, each and every publication and patent document cited in this disclosure is incorporated herein by reference as if each such publication or document was specifically and individually indicated to be incorporated herein by reference. Citation of publications and patent documents is not intended as an indication that any such document is pertinent prior art, nor does it constitute an admission as to its contents or date.

What is claimed is:

1. An engineered RNA ligase, or a functional fragment thereof, comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 346 of an even-numbered SEQ ID NO. of SEQ ID NOs: 14 and 24-582, or to a reference sequence corresponding to an even-numbered SEQ ID NO. of SEQ ID NOs: 14 and 24-582, wherein the amino acid sequence comprises one or more substitutions relative to the reference sequence corresponding to residues 12 to 346 of SEQ ID NO: 14, 42, or 204, or relative to the reference sequence corresponding to SEQ ID NO: 14, 42, or 204.

2. The engineered RNA ligase of claim 1, comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 14, 42, or 204, or to the reference sequence corresponding to SEQ ID NO: 14, 42, or 204, wherein the amino acid sequence comprises one or more substitutions relative to the reference sequence corresponding to residues 12 to 346 of SEQ ID NO: 14, 42, or 204, or relative to the reference sequence corresponding to SEQ ID NO: 14, 42, or 204.

3. The engineered RNA ligase of claim 1, comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 14, or to the reference sequence corresponding to SEQ ID NO: 14, wherein the amino acid sequence comprises one or more substitutions relative to the reference sequence corresponding to residues 12 to 346 of SEQ ID NO: 14, or relative to the reference sequence corresponding to SEQ ID NO: 14.

4. The engineered RNA ligase of claim 1, comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 42 or 204, or to the reference sequence corresponding to SEQ ID NO: 42 or 204, wherein the amino acid sequence comprises one or more substitutions relative to the reference sequence corresponding to residues 12 to 346 of SEQ ID NO: 14, or relative to the reference sequence corresponding to SEQ ID NO: 14.

5. The engineered RNA ligase of claim 1, comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 24-582, or to the reference sequence corresponding to an even-numbered SEQ ID NO. of SEQ ID NOs: 24-582, wherein the amino acid sequence comprises one or more substitutions relative to the reference sequence corresponding to residues 12 to 346 of SEQ ID NO: 14, or relative to the reference sequence corresponding to SEQ ID NO: 14.

6. The engineered RNA ligase of claim 1, comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 24-128, or to the reference sequence corresponding to an even-numbered SEQ ID NO. of SEQ ID NOs: 24-128, wherein the amino acid sequence comprises one or more substitutions relative to the reference sequence corresponding to residues 12 to 346 of SEQ ID NO: 14, or relative to the reference sequence corresponding to SEQ ID NO: 14.

7. The engineered RNA ligase of any one of claims 1-6, wherein the amino acid sequence of the engineered RNA ligase comprises at least a substitution at amino acid position 2, 14, 15, 18, 33, 39, 63, 69, 81, 83, 85, 86, 89, 95, 116, 117, 119, 138, 142, 144, 149, 154, 156, 159, 171, 175, 177, 181, 185, 186, 195, 202, 212, 214, 224, 226, 230, 247, 256, 257, 260, 266, 275, 280, 283, 285, 288, 291, 296, 303, 306, 307, 310, 314, 315, 316, 317, 326, 330, 331, 333, 334, 335, 337, 339, 342, or 345, or combinations thereof, wherein the amino acid positions are relative to the reference sequence corresponding to residues 12 to 346 of SEQ ID NO: 14, or relative to the reference sequence corresponding to SEQ ID NO: 14.

8. The engineered RNA ligase of any one of claims 1-6, wherein the amino acid sequence of the engineered RNA ligase comprises at least a substitution or amino acid residue 2G/V/E, 14K, 15L/E/M/P/Y, 18I, 33L, 39Q/S, 63V, 69D, 81Y, 85L, 86G, 89H/I/M/W, 95V, 116N, 117D, 119T, 138R, 142R, 144W, 149R/T, 154C, 156A/L/N/Q/T, 159F, 171E, 175K, 177P, 181D, 183V, 185G/K, 186D/R, 195G, 202Y, 212I, 214I, 224Y, 226I, 230D, 247K, 256A, 257L, 260S, 266L, 275N, 280G/N, 283I, 285K, 288E, 291P, 296K, 303Q, 306L, 307E/Q, 310K, 314W, 315S/T, 316L, 317A, 326R, 330M/R, 331R/W, 333V, 334R, 335D/H, 337L, 339P, 342I, or 345V, or combinations thereof, wherein the amino acid positions are relative to the reference sequence corresponding to residues 12 to 346 of SEQ ID NO: 14, or relative to the reference sequence corresponding to SEQ ID NO: 14.

9. The engineered RNA ligase of any one of claims 1-6, wherein the amino acid sequence of the engineered RNA ligase comprises at least a substitution at amino acid position 95 or 177, or combination thereof, wherein the amino acid positions are relative to the reference sequence corresponding to residues 12 to 346 of SEQ ID NO: 14, or relative to the reference sequence corresponding to SEQ ID NO: 14.

10. The engineered RNA ligase of any one of claims 1-6, wherein the amino acid sequence of the engineered RNA ligase comprises at least a substitution or amino acid residue 95V or 177P, or combination thereof, wherein the amino acid positions are relative to the reference sequence corresponding to residues 12 to 346 of SEQ ID NO: 14, or relative to the reference sequence corresponding to SEQ ID NO: 14.

11. The engineered RNA ligase of any one of claims 1-6, wherein the amino acid sequence of the engineered RNA ligase comprises at least a substitution at amino acid position 15, 95, 149, 154, 156, 177, 186, 195, 224, or 226, or combinations thereof, wherein the amino acid positions are relative to the reference sequence corresponding to residues 12 to 346 of SEQ ID NO: 14, or relative to the reference sequence corresponding to SEQ ID NO: 14.

12. The engineered RNA ligase of any one of claims 1-6, wherein the amino acid sequence of the engineered RNA ligase comprises at least a substitution or amino acid residue 15L/E/M/P/Y, 95V, 149R/T, 154C, 156A/L/N/Q/T, 177P, 186D/R, 195G, 224Y, or 226I, or combinations thereof, wherein the amino acid positions are relative to the reference sequence corresponding to residues 12 to 346 of SEQ ID NO: 14, or relative to the reference sequence corresponding to SEQ ID NO: 14.

13. The engineered RNA ligase of any one of claims 1-6, wherein the amino acid sequence of the engineered RNA ligase comprises at least a substitution or amino acid residue 15Y, 95V, 149R, 154C, 156T, 177P, 186R, 195G, 224Y, or 226I, or combinations thereof, wherein the amino acid positions are relative to the reference sequence corresponding to residues 12 to 346 of SEQ ID NO: 14, or relative to the reference sequence corresponding to SEQ ID NO: 14.

14. The engineered RNA ligase of any one of claims 1-6, wherein the amino acid sequence of the engineered RNA ligase comprises at least a substitution or substitution set at amino acid position(s) 177/260/307/345, 171/175/183/280/283/303, 95/159/177/260/288, 175/183/247/280/283/296, 177/307, 81/171/183/335, 175/183, 81/175/183/280/296/335/339, 81/183/247/266, 95/177, 171/280/283/296/303, 85/177/260/288/333/345, 177/291/307/333, 81/171/175/183/316/337, 81/171/266/280/283/335/339, 177, 95/159/177/260/280/288/306/345, 171/175/335, 177/260/30/333, 14/95/177/288/307, 95/177/345, 95/159/177/260/285/288, 266/296, 81/283/335/337, 95/159/177/260/342/345, 63/171/175/183/266/280/296/316, 280/335/337, 81/171/280, 81/171/175/296/316, 171/175/316/335, 171/280/283/296/316, 171/283/335, 63/171/175/183/266/283/303/316, 171/247/266/283/296/303/316/335, 63/81/171/175/183/266/280/283/337, 81/171/303/316/335, 288/307, 14/95/159/177/345, 81/171/175/183/247/266/280/283, 81/247/266, 280, 85/177/260, 63/183, 175/183/247/280/283/316, 291, 247, 171/280/283, 81/171/175/280/316, 333, 171/296/316, 81/171, or 280/316/335, wherein the amino acid positions are relative to the reference sequence corresponding to residues 12 to 346 of SEQ ID NO: 14, or relative to the reference sequence corresponding to SEQ ID NO: 14.

15. The engineered RNA ligase of any one of claims 1-6, wherein the amino acid sequence of the engineered RNA ligase comprises at least a substitution or substitution set 177P/260S/307E/345V, 171E/175K/183V/280N/283I/303Q, 95V/159F/177P/260S/288E, 175K/183V/247K/280N/283I/296K, 177P/307E, 81Y/171E/183V/335D, 175K/183V, 81Y/175K/183V/280N/296K/335D/339P, 81Y/183V/247K/266L, 95V/177P, 171E/280N/283I/296K/303Q, 85L/177P/260S/288E/333V/345V, 177P/291P/307E/333V, 81Y/171E/175K/183V/316L/337L, 81Y/171E/266L/280N/283I/335D/339P, 177P, 95V/159F/177P/260S/280N/288E/306L/345V, 171E/175K/335D, 177P/260S/306L/333V, 14K/95V/177P/288E/307E, 95V/177P/345V, 95V/159F/177P/260S/285K/288E, 266L/296K, 81Y/283I/335D/337L, 95V/159F/177P/260S/342I/345V, 63V/171E/175K/183V/266L/280N/296K/316L, 280N/335D/337L, 81Y/171E/280N, 81Y/171E/175K/296K/316L, 171E/175K/316L/335D, 171E/280N/283I/296K/316L, 171E/283I/335D, 63V/171E/175K/183V/266L/283I/303Q/316L, 171E/247K/266L/283I/296K/303Q/316L/335D, 63V/81Y/171E/175K/183V/266L/280N/283I/337L, 81Y/171E/303Q/316L/335D, 288E/307E, 14K/95V/159F/177P/345V, 81Y/171E/175K/183V/247K/266L/280N/283I, 81Y/247K/266L, 280N, 85L/177P/260S, 63V/183V, 175K/183V/247K/280N/283I/316L, 291P, 247K, 171E/280N/283I, 81Y/171E/175K/280N/316L, 333V, 171E/296K/316L, 81Y/171E, or 280N/316L/335D, wherein the amino acid positions are relative to the reference sequence corresponding to residues 12 to 346 of SEQ ID NO: 14, or relative to the reference sequence corresponding to SEQ ID NO: 14.

16. The engineered RNA ligase of claim 1, wherein the amino acid sequence of the engineered RNA ligase comprises at least one substitution provided in Tables 5.1, 6.1, 7.1, 8.1, 9.1, 10.1, 11.1, and 12.1, wherein the amino acid positions are relative to the reference sequence corresponding to residues 12 to 346 of SEQ ID NO: 14, or relative to the reference sequence corresponding to SEQ ID NO: 14.

17. The engineered RNA ligase of claim 1, wherein the amino acid sequence of the engineered RNA ligase comprises at least a substitution or substitution set of an RNA ligase variant provided in Tables 5.1, 6.1, 7.1, 8.1, 9.1, 10.1, 11.1, and 12.1, wherein the amino acid positions are relative to the reference sequence corresponding to residues 12 to 346 of SEQ ID NO: 14, or relative to the reference sequence corresponding to SEQ ID NO: 14.

18. The engineered RNA ligase of claim 1, comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 42 or 204, or to the reference sequence corresponding to SEQ ID NO: 42 or 204.

19. The engineered RNA ligase of claim 1, comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 24-582, or to the reference sequence corresponding to an even-numbered SEQ ID NO. of SEQ ID NOs: 24-582.

20. The engineered RNA ligase of claim 1, comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 42 or 204, or to the reference sequence corresponding to SEQ ID NO: 42 or 204, wherein the amino acid sequence comprises one or more substitutions relative to the reference sequence corresponding to residues 12 to 346 of SEQ ID NO: 42 or 204, or relative to the reference sequence corresponding to SEQ ID NO: 42 or 204.

21. The engineered RNA ligase of claim 1, comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 24-582, or to the reference sequence corresponding to an even-numbered SEQ ID NO. of SEQ ID NOs: 24-582, wherein the amino acid sequence comprises one or more substitutions relative to the reference sequence corresponding to residues 12 to 346 of SEQ ID NO: 42 or 204, or relative to the reference sequence corresponding to SEQ ID NO: 42 or 204.

22. The engineered RNA ligase of claim 20 or 21, wherein the amino acid sequence of the engineered RNA ligase comprises at least a substitution at amino acid position 2, 14, 15, 18, 33, 39, 63, 69, 81, 83, 85, 86, 89, 95, 116, 117, 119, 138, 142, 144, 149, 154, 156, 159, 171, 175, 177, 181, 185, 186, 195, 202, 212, 214, 224, 226, 230, 247, 256, 257, 260, 266, 275, 280, 283, 285, 288, 291, 296, 303, 306, 307, 310, 314, 315, 316, 317, 326, 330, 331, 333, 334, 335, 337, 339, 342, or 345, or combinations thereof, wherein the amino acid positions are relative to the reference sequence corresponding to residues 12 to 346 of SEQ ID NO: 42 or 204, or relative to the reference sequence corresponding to SEQ ID NO: 42 or 204.

23. The engineered RNA ligase of claim 20 or 21, wherein the amino acid sequence of the engineered RNA ligase comprises at least a substitution or an amino acid residue 2G/V/E, 14K, 15E/L/M/P/Y, 18I, 33L, 39Q/S, 63V, 69D, 81Y, 85L, 86G, 89H/I/M/W, 95V, 116N, 117D, 119T, 138R, 142R, 144W, 149R/T, 154C, 156A/L/N/Q/T, 159F, 171E, 175K, 177P, 181D, 183V, 185G/K, 186D/R, 195G, 202Y, 212I, 214I, 224Y, 226I, 230D, 247K, 256A, 257L, 260S, 266L, 275N, 280G/N, 283I, 285K, 288E, 291P, 296K, 303Q, 306L, 307E/Q, 310K, 314W, 315S/T, 316L, 317A, 326R, 330M/R, 331R/W, 333V, 334R, 335D/H, 337L, 339P, 342I, or 345V, or combinations thereof, wherein the amino acid positions are relative to the reference sequence corresponding to residues 12 to 346 of SEQ ID NO: 42 or 204, or relative to the reference sequence corresponding to SEQ ID NO: 42 or 204.

24. The engineered RNA ligase of claim 20 or 21, wherein the amino acid sequence of the engineered RNA ligase comprises at least a substitution at amino acid position 95 or 177, or combination thereof, wherein the amino acid positions are relative to the reference sequence corresponding to residues 12 to 346 of SEQ ID NO: 42 or 204, or relative to the reference sequence corresponding to SEQ ID NO: 42 or 204.

25. The engineered RNA ligase of claim 20 or 21, wherein the amino acid sequence of the engineered RNA ligase comprises at least a substitution or amino acid residue 95V or 177P, or combination thereof, wherein the amino acid positions are relative to the reference sequence corresponding to residues 12 to 346 of SEQ ID NO: 42 or 204, or relative to the reference sequence corresponding to SEQ ID NO: 42 or 204.

26. The engineered RNA ligase of claim 20 or 21, wherein the amino acid sequence of the engineered RNA ligase comprises at least a substitution at amino acid position 15, 95, 149, 154, 156, 177, 186, 195, 224, or 226, or combinations thereof, wherein the amino acid positions are relative to the reference sequence corresponding to residues 12 to 346 of SEQ ID NO: 42 or 204, or relative to the reference sequence corresponding to SEQ ID NO: 42 or 204.

27. The engineered RNA ligase of claim 20 or 21, wherein the amino acid sequence of the engineered RNA ligase comprises at least a substitution 15Y, 95V, 149R, 154C, 156T, 177P, 186R, 195G, 224Y, or 226I, or combinations thereof, wherein the amino acid positions are relative to the reference sequence corresponding to residues 12 to 346 of SEQ ID NO: 42 or 204, or relative to the reference sequence corresponding to SEQ ID NO: 42 or 204.

28. The engineered RNA ligase of claim 20, comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 42, or to the reference sequence corresponding to SEQ ID NO: 42, wherein the amino acid sequence comprises one or more substitutions relative to the reference sequence corresponding to residues 12 to 346 of SEQ ID NO: 42, or relative to the reference sequence corresponding to SEQ ID NO: 42.

29. The engineered RNA ligase of claim 21, comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 130-478, or to the reference sequence corresponding to an even-numbered SEQ ID NO. of SEQ ID NOs: 130-478, wherein the amino acid sequence comprises one or more substitutions relative to the reference sequence corresponding to residues 12 to 346 of SEQ ID NO: 42, or relative to the reference sequence corresponding to SEQ ID NO: 42.

30. The engineered RNA ligase of claim 28 or 29, wherein the amino acid sequence of the engineered RNA ligase comprises a substitution or substitution set at amino acid positions(s) 149/156/195, 15/149/186/224/317, 15/156/195/224/226/317/331, 15/195/317/331, 149/224/331, 15/195/224/226/317/331, 15/33/86/149/154/195/317, 15/86/154/156/186/195/224/226/331, 15/149/224/317/331, 15/154/156/186/317/331, 15/33/149/224/226/331, 15/186/224/226/317/331, 15/33/149/186/224/331, 195/224/331, 15/86/156/186/195/226/317/331, 15/195/22/331, 156/186/224/226/331, 15/149/154/156/224/226, 33/156/186/195/331, 15/86/149/154/156/317/331, 15/186/317/331, 15/86/149/154/195/331, 15/33/149/154/156/226, 15/149/154/186/317/331, 195/331, 15/86/156/186/195/331, 149/156/224/226/331, 15/149/154/156/186/317/331, 15/154/186/195, 15/86/195/224/226, 230, 15/33/156/331, 15/33/156/195/224/226, 33/149/156/317, 15/33/186/195, 334, 15/154/156/224/317, 15/149/186/331, 15/86/149/186/195/317, 15/33/154/195, 15/156/186/331, 15/149/186/317, 15/33/156/186/214/224/331, 15/86/186/224/226/317, 15/154/331, 15/149/154/226, 15/33/195/226, 15/224/317/331, 15/86/186/195/317, 226/317/331, 330, 89, 15/149/186, 156/195/331, 156, 15/86/156/186/331, 275, 15/33/186/224, 15/186/317, 15/33/186/226/317, 15/149/154/156/331, 15/154/226/317, 15/86/156/186/195/317, 257, 15/154/156/186, 33/186/224, 335, 15/86/156/186/195/224/331, 154/156/317, 39, 2, 15/224/226/331, 15/33/149/224, 89/307, 15/154/156/317, 15/186/331, 15/33/154/317, 15/33/195/224/317, 15/156/224/226, 154/186/331, 185, 15/18/149/186/195, 86/149/154/156, 15/33/149/331, 326, 202, 119, 212, 15/33/156/317, 280, 142, 15/149, 15/33/86/156/195, 315, 256, 15/33/156/186, 138, 117, 116, 316, 69, 310, 317, 15/195/331, 15/33/331, 314, or 144, wherein the amino acid positions are relative to the reference sequence corresponding to residues 12 to 346 of SEQ ID NO: 42, or relative to the reference sequence corresponding to SEQ ID NO: 42.

31. The engineered RNA ligase of claim 28 or 29, wherein the amino acid sequence of the engineered RNA ligase comprises a substitution or substitution set 149R/156T/195G, 15M/149T/186R/224Y/317A, 15P/156T/195G/224Y/226I/317A/331L, 15M/195G/317A/331R, 149R/224Y/331R, 15E/195G/224Y/226I/317A/331W, 15P/33L/86G/149R/154C/195G/317A, 15M/86G/154C/156T/186R/195G/224Y/226I/331L, 15P/149R/224Y/317A/331R, 15M/154C/156T/186R/317A/331R, 15M/33L/149R/224Y/226I/331W, 15E/186R/224Y/226I/317A/331R, 15Y/33L/149R/186R/224Y/331R, 195G/224Y/331R, 15P/86G/156T/186R/195G/226I/317A/331L, 15M/195G/226/331W, 156T/186R/224Y/226I/331R, 15Y/149R/154C/156T/224Y/226I, 33L/156T/186R/195G/331L, 15P/86G/149R/154C/156T/317A/331R, 15M/186D/317A/331R, 15P/86G/149R/154C/195G/331L, 15Y/33L/149T/154C/156T/226I, 15P/149R/154C/186R/317A/331L, 195G/331R, 15M/86G/156T/186R/195G/331W, 149R/156T/224Y/226I/331W, 15P/149R/154C/156T/186R/317A/331L, 15Y/154C/186D/195G, 15E/86G/195G/224Y/226I, 230D, 15P/33L/156T/331R, 15L/33L/156T/195G/224Y/226I, 33L/149R/156T/317A, 15E/33L/186R/195G, 334R, 15E/154C/156T/224Y/317A, 15Y/149R/186R/331W, 15Y/86G/149T/186R/195G/317A, 15P/33L/154C/195G, 15P/154C/156T/186R/317A/331R, 15M/156T/186R/331R, 15Y/149T/186D/317A, 15E/33L/156T/186R/214I/224Y/331W, 15E/86G/186R/224Y/226I/317A, 15P/154C/331R, 15Y/149T/154C/226I, 15Y/33L/195G/226I, 15M/224Y/317A/331W, 15E/86G/186R/195G/317A, 226I/317A/331L, 330R, 89W, 15P/149R/186D/331L, 15P/149R/186R, 89M, 156T/195G/331L, 156A, 15E/86G/156T/186R/331R, 195G/331W, 275N, 15P/33L/186R/224Y, 15E/186R/317A, 15M/33L/186R/226I/317A, 89G, 15Y/149R/154C/156T/331W, 15Y/154C/226I/317A, 15Y/86G/156T/186R/195G/317A, 257L, 15Y/154C/156T/186D, 33L/186R/224Y, 335H, 15Y/86G/156T/186R/195G/224Y/331W, 154C/156T/317A, 39Q, 2G, 15M/224Y/226I/331R, 15M/33L/149R/224Y, 89I/307Q, 15M/154C/156T/317A, 15E/186R/331W, 15P/33L/154C/317A, 89H, 15Y/33L/195G/224Y/317A, 15E/156T/224Y/226I, 154C/186R/331L, 185G, 15M/18I/149R/186R/195G, 86G/149R/154C/156T, 15P/33L/149R/331L, 326R, 202Y, 119T, 212I, 2V, 15E/149R/154C/156T/331L, 15P/33L/156T/317A, 15Y/154C/156T/317A, 156L, 335D, 156T, 2E, 280G, 142R, 15E/149R, 156Q, 15E/33L/86G/156T/195G, 315T, 185K, 39S, 256A, 15Y/33L/156T/186D, 138R, 117D, 116N, 316L, 15M/149R, 69D, 156N, 310K, 317A, 15Y/195G/331L, 15M/33L/331L, 330M, 314W, 144W, 15Y/149R, or 315S, wherein the amino acid positions are relative to the reference sequence corresponding to residues 12 to 346 of SEQ ID NO: 42, or relative to the reference sequence corresponding to SEQ ID NO: 42.

32. The engineered RNA ligase of claim 28 or 29, wherein the amino acid sequence of the engineered RNA ligase comprises at least a substitution set at amino acid positions(s) 15/149/154/156/186/195/331, 149/154/186/195/224/317/331, 15/33/149/154/186/195, 149/156/186/195/224/226/317, 15/149/156/195/224/226, 15/33/86/149/154/156/186/195/226/331, 15/149/156/186/195/224/317/331, 15/33/149/154/195/317, 15/149/154/195/224/317, 15/149/156/186/224/226/317/331, 15/33/149/154/156/186/195/224/226/317/331, 149/195/224, 86/149/154/156/186/195/331, 15/33/149/156/195/224/317/331, 15/149/154/156/186/195/317/331, 15/33/149/154/156/186/224/331, 15/149/154/156/195/224, 15/149/154/156/195/224/226, 15/149/154/156/186/195/224/317, 33/149/156/186/224/331, 15/33/86/149/195/224/331, 15/86/149/154/156/186/195/331, 149/186/195/331, 15/154/156/186/226/331, 15/149/154/186/195/224/226, 15/149/154/156/195/224/331, 15/33/149/154/156/195/317/331, 15/33/154/156/186/195/224/317/331, 15/149/186/195/224/331, 15/33/149/154/156/195/224/331, 15/149/154/156/224/226/317/331, 15/33/149/156/195/331, 15/149/154/156/186/224/226/331, 154/156/195/224/226, 15/33/86/149/154/156/186/195/224/317/331, 15/149/154/156/186/195/224/226, 15/33/149/195/317/331, 15/149/154/224/331, 149/154/156/195/224/226/331, 15/149/154/195/331, 15/149/154/156/186/195, 15/33/149/154/156/186/195, 15/33/86/149/154/195/224/317/331, or 149/186/195/224/226/331, wherein the amino acid positions are relative to the reference sequence corresponding to residues 12 to 346 of SEQ ID NO: 42, or relative to the reference sequence corresponding to SEQ ID NO: 42.

33. The engineered RNA ligase of claim 28 or 29, wherein the amino acid sequence of the engineered RNA ligase comprises at least a substitution set 15M/149R/154C/156T/186R/195G/331R, 149R/154C/186D/195G/224Y/317A/331R, 15M/33L/149T/154C/186R/195G, 149R/156T/186R/195G/224Y/226I/317A, 15Y/149R/156T/195G/224Y/226I, 15Y/33L/86G/149T/154C/156T/186R/195G/226I/331W, 15Y/149R/156T/186R/195G/224Y/317A/331W, 15M/33L/149T/154C/195G/317A, 15M/149R/154C/195G/224Y/317A, 15Y/149R/156T/186D/224Y/226I/317A/331R, 15M/149T/154C/156T/186D/195G/331L, 15Y/33L/149R/154C/156T/186R/195G/224Y/226I/317A/331R, 149R/195G/224Y, 86G/149T/154C/156T/186R/195G/331L, 15P/33L/149R/156T/195G/224Y/317A/331L, 15P/149T/154C/156T/186D/195G/317A/331L, 15Y/33L/149T/154C/156T/186D/224Y/331L, 15P/149R/156T/186R/195G/224Y/317A/331W, 15Y/149R/154C/156T/195G/224Y, 15M/149T/154C/156T/195G/224Y/226I, 15E/149R/154C/156T/186R/195G/224Y/317A, 33L/149T/156T/186R/224Y/331L, 15Y/33L/86G/149R/195G/224Y/331W, 15E/86G/149R/154C/156T/186R/195G/331W, 149R/186R/195G/331W, 15E/154C/156T/186D/226I/331R, 15Y/149T/154C/186R/195G/224Y/226I, 15P/149R/154C/156T/195G/224Y/331W, 15E/33L/149R/154C/156T/195G/317A/331L, 15E/33L/154C/156T/186R/195G/224Y/317A/331L, 15P/149R/186D/195G/224Y/331L, 15P/33L/149R/154C/156T/195G/224Y/331W, 15P/149T/154C/156T/224Y/226I/317A/331W, 15P/33L/149T/156T/195G/331W, 15P/149T/154C/156T/186D/224Y/226I/331L, 154C/156T/195G/224Y/226I, 15Y/33L/86G/149R/154C/156T/186D/195G/224Y/317A/331W, 15Y/149R/154C/156T/186R/195G/224Y/226I, 15P/33L/149R/195G/317A/331L, 15E/149T/154C/224Y/331R, 149T/154C/156T/195G/224Y/226I/331R, 15M/149R/154C/195G/331L, 15Y/149R/154C/156T/186R/195G, 15E/149T/154C/156T/186R/195G/331R, 15M/33L/149T/154C/156T/186R/195G, 15P/33L/86G/149R/154C/195G/224Y/317A/331R, or 149R/186D/195G/224Y/226I/331R, wherein the amino acid positions are relative to the reference sequence corresponding to residues 12 to 346 of SEQ ID NO: 42, or relative to the reference sequence corresponding to SEQ ID NO: 42.

34. The engineered RNA ligase of claim 19, comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of SEQ ID NO: 204, or to the reference sequence corresponding to SEQ ID NO: 204, wherein the amino acid sequence comprises one or more substitutions relative to the reference sequence corresponding to residues 12 to 346 of SEQ ID NO: 204, or relative to the reference sequence corresponding to SEQ ID NO: 204.

35. The engineered RNA ligase of claim 20, comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 346 of an even-numbered SEQ ID NO. of SEQ ID NOs: 480-582, to the reference sequence corresponding to an even-numbered SEQ ID NO. of SEQ ID NOs: 480-582, wherein the amino acid sequence comprises one or more substitutions relative to the reference sequence corresponding to residues 12 to 346 of SEQ ID NO: 204, or relative to the reference sequence corresponding to SEQ ID NO: 204.

36. The engineered RNA ligase of claim 34 or 35, wherein the amino acid sequence of the engineered RNA ligase comprises at least a substitution or substitution set at amino acid position(s) 2/39/69/144/156/316/330, 2/39/69/119/185/316, 69/212/256/330, 39/119/256/316/326/330, 69/116/119/185/330, 2/39/119/144/156/159/202/256/316/326/330, 81/183/185/230, 2/39/69/116/144/156/185, 2/14/39/69/116/119/159/185/256/326, 2/116/119/316, 116/119/144/185/314/316, 39/69/116/144/181/185/256/330, 2/39/69/119/144/156/185/314, 14/39/69/144/156/212/256/314/316/326/330, 39/69/144/156/185/316/330, 156/185/334/335, 39/63/156/230/257/275/330/335, 156/266/316/330/334/335, 39/63/156/183/185/230/330/334, 69/144/316, 116/119/156/202/212/326/330, 39/69/119/138/181/185/316/326, 39/81/156/230, 2/14/69/116/119/144/159/326/330, 39/156/334/345, 230/266/334, 2/116/144/156/159, 156/183/185/230, 2/14/116/316/326/330, 39/63/156/230/316/345, 69/138/144/159/185/202/316, 39/156/257/266/316/334, 156/257/275/334, 183, 89/156/185/230/316/345, 2/14/119, 144/156/316/330, 39/81/185/316/334, 89/230/266/345, 39/156/230/275/316, 39/156/230/266/334/335, 156/185/275/316/330, 119/156/202, 230/257/275/316/330/345, 230, 81/156/330/335/345, 156/183/230/266, 39/185, 39/81/183/275/316/334/345, 156/230/257/316, 39/116/156, or 183/185/257/275/316/330/334, wherein the amino acid positions are relative to the reference sequence corresponding to residues 12 to 346 of SEQ ID NO: 204, or relative to the reference sequence corresponding to SEQ ID NO: 204.

37. The engineered RNA ligase of claim 34 or 35, wherein the amino acid sequence of the engineered RNA ligase comprises at least a substitution or substitution set 2E/39S/69D/144W/156L/316L/330M, 2V/39S/69D/119T/185K/316L, 69D/212I/256A/330M, 39S/119T/256A/316L/326R/330M, 69D/116N/119T/185K/330M, 2E/39S/119T/144W/156N/159F/202Y/256A/316L/326R/330M, 81Y/183V/185G/230D, 2V/39S/69D/116N/144W/156L/185K, 2E/14K/39S/69D/116N/119T/159F/185K/256A/326R, 2E/116N/119T/316L, 116N/119T/144W/185K/314W/316L, 39S/69D/116N/144W/181D/185K/256A/330M, 2E/39S/69D/119T/144W/156L/185K/314W, 14K/39S/69D/144W/156N/212I/256A/314W/316L/326R/330M, 39S/69D/144W/156L/185K/316L/330M, 156Q/185G/334R/335H, 39Q/63V/156Q/230D/257L/275N/330R/335H, 156Q/266L/316L/330R/334R/335H, 39Q/63V/156Q/183V/185G/230D/330R/334R, 69D/144W/316L, 116N/119T/156L/202Y/212I/326R/330M, 39S/69D/119T/138R/181D/185K/316L/326R, 39Q/81Y/156Q/230D, 2V/14K/69D/116N/119T/144W/159F/326R/330M, 39Q/156A/334R/345V, 230D/266L/334R, 2V/116N/144W/156L/159F, 156A/183V/185G/230D, 2E/14K/116N/316L/326R/330M, 39Q/63V/156A/230D/316L/345V, 69D/138R/144W/159F/185K/202Y/316L, 39Q/156Q/257L/266L/316L/334R, 156Q/257L/275N/334R, 183V, 89W/156A/185G/230D/316L/345V, 2E/14K/119T, 144W/156L/316L/330M, 39Q/81Y/185G/316L/334R, 89H/230D/266L/345V, 39Q/156Q/230D/275N/316L, 39Q/156Q/230D/266L/334R/335H, 156Q/185G/275N/316L/330R, 119T/156N/202Y, 230D/257L/275N/316L/330R/345V, 230D, 81Y/156Q/330R/335H/345V, 156A/183V/230D/266L, 39S/185K, 39Q/81Y/183V/275N/316L/334R/345V, 156A/230D/257L/316L, 39S/116N/156N, or 183V/185G/257L/275N/316L/330R/334R, wherein the amino acid positions are relative to the reference sequence corresponding to residues 12 to 346 of SEQ ID NO: 204, or relative to the reference sequence corresponding to SEQ ID NO: 204.

38. The engineered RNA ligase of claim 1, wherein the amino acid sequence of the engineered RNA ligase comprises at least one substitution provided in Tables 6.1, 7.1, 8.1, 9.1, 10.1, 11.1, and 12.1, wherein the amino acid positions are relative to the reference sequence corresponding to residues 12 to 346 of SEQ ID NO: 42 or 204, or relative to the reference sequence corresponding to SEQ ID NO: 42 or 204.

39. The engineered RNA ligase of claim 1, wherein the amino acid sequence of the engineered RNA ligase comprises at least a substitution or substitution set of an RNA ligase variant provided in Tables 6.1, 7.1, 8.1, 9.1, 10.1, 11.1, and 12.1, wherein the amino acid positions are relative to the reference sequence corresponding to residues 12 to 346 of SEQ ID NO: 42 or 204, or relative to the reference sequence corresponding to SEQ ID NO: 42 or 204.

40. The engineered RNA ligase of claim 1, comprising an amino acid sequence having least 80%, 81%, 82%, 83%, 84%, 85%, 86%, 87%, 88%, 89%, 90%, 91%, 92%, 93%, 94%, 95%, 96%, 97%, 98%, 99% or more sequence identity to a reference sequence comprising a substitution or substitution set provided in Tables 5.1, 6.1, 7.1, 8.1, 9.1, 10.1, 11.1, and 12.1, wherein the amino acid positions are relative to the reference sequence corresponding to residues 12 to 346 of SEQ ID NO: 14, 42, or 204, or relative to the reference sequence corresponding to SEQ ID NO: 14, 42, or 204.

41. The engineered RNA ligase of claim 1, wherein the RNA ligase comprises an amino acid sequence comprising residues 12 to 346 of an even numbered SEQ ID NO. of SEQ ID NOs: 24-582, or an amino acid sequence comprising an even numbered SEQ ID NO. of SEQ ID NOs: 24-582, optionally wherein the amino acid sequence has 1, 2, 3, 4, 5, 6, 7, 8, 9, or up to 10 substitutions.

42. The engineered RNA ligase of any one of claims 1-6, wherein the engineered RNA ligase comprises a fusion polypeptide.

43. The engineered RNA ligase of any one of claims 1-6, wherein the engineered RNA ligase has RNA ligase 2 activity.

44. The engineered RNA ligase of any one of claims 1-6, having at least one improved property as compared to a reference RNA ligase.

45. The engineered RNA ligase of claim 44, wherein the improved property is selected from i) increased activity, ii) increased stability, iii) increased thermostability, iv) increased product yield, v) increased activity on polynucleotides ligase substrates with phosphorothioate internucleotide linkages, vi) increased activity on oligonucleotides with 2′-modifications, vii) increased substrate tolerance, or any combination of i), ii), iii), iv), v), vi), and vii), compared to a reference RNA ligase.

46. The engineered RNA ligase of claim 44, wherein the reference RNA ligase has the sequence corresponding to residues 12 to 346 of SEQ ID NO: 14, 42, or 204, or the sequence corresponding to SEQ ID NO: 14, 42, or 204.

47. The engineered RNA ligase of claim 44, wherein the reference RNA ligase has the sequence corresponding to residues 12 to 346 of SEQ ID NO: 14, or the sequence corresponding to SEQ ID NO: 14.

48. The engineered RNA ligase of any one of claims 1-6, 16-21, 28, 29, 34, 35, and 38-41, wherein the engineered RNA ligase is purified.

49. A recombinant polynucleotide comprising a polynucleotide sequence encoding an engineered RNA ligase of any one of claims 1-6, 16-21, 28, 29, 34, 35, and 38-41.

50. The recombinant polynucleotide of claim 49, wherein the polynucleotide sequence has least 70%, 75%, 75%, 80%, 81%, 82%, 83%, 84%, 85%, 90%, 91%, 92%, 93%, 94%, 95%, 96%, 97%, 98%, 99%, or more sequence identity to a reference polynucleotide sequence corresponding to nucleotide residues 34 to 1038 of SEQ ID NO: 13, 41, or 203, or to a reference polynucleotide sequence corresponding to SEQ ID NO: 13, 41, or 203, wherein the recombinant polynucleotide encodes an RNA ligase.

51. The recombinant polynucleotide of claim 49, wherein the polynucleotide sequence has at least 70%, 75%, 80%, 81%, 82%, 83%, 84%, 85%, 90%, 91%, 92%, 93%, 94%, 95%, 96%, 97%, 98%, 99%, or more sequence identity to a reference polynucleotide sequence corresponding to nucleotide residues 34 to 1038 of an odd numbered SEQ ID NO. of SEQ ID NOs: 23-581, or to a reference polynucleotide sequence corresponding an odd numbered SEQ ID NO. of SEQ ID NOs: 23-581, wherein the recombinant polynucleotide encodes an RNA ligase.

52. The recombinant polynucleotide of claim 49, wherein the polynucleotide sequence is codon-optimized for expression of the encoded engineered RNA ligase.

53. The recombinant polynucleotide of claim 49, comprising
(a) a polynucleotide sequence comprising nucleotide residues 34 to 1038 of SEQ ID NO. 13, 41, or 203, or a polynucleotide sequence comprising SEQ ID NOs: 13, 41, or 203, or
(b) a polynucleotide sequence comprising nucleotide residues 34 to 1038 of an odd numbered SEQ ID NO. of SEQ ID NOs: 23-581, or a polynucleotide sequence comprising an odd numbered SEQ ID NO. of SEQ ID NOs: 23-581.

54. An expression vector comprising a recombinant polynucleotide of claim 49.

55. The expression vector of claim 54, wherein the polynucleotide is operably linked to a control sequence.

56. The expression vector of claim 55, wherein the control sequence comprises at least a promoter.

57. A host cell comprising an expression vector of claim 54.

58. The host cell of claim 57, comprising a prokaryotic cell or eukaryotic cell.

59. The host cell of claim 58, comprising a bacterial cell, fungal cell, insect cell, or mammalian cell.

60. A method of producing an engineered RNA ligase polypeptide in a host cell comprising culturing a host cell of claim 57, under suitable culture conditions such that the engineered RNA ligase is produced.

61. The method of claim 60, further comprising recovering the engineered RNA ligase from the culture and/or host cells.

62. The method of claim 60, further comprising purifying the engineered RNA ligase.

63. A composition comprising an RNA ligase of any one of claims 1-6, 16-21, 28, 29, 34, 35, and 38-41.

64. The composition of claim 63, further comprising one or more of a buffer, ATP or dATP, and one or more polynucleotide substrates for the RNA ligase.

65. A method of ligating at least a first polynucleotide strand and a second polynucleotide strand, comprising contacting a first polynucleotide strand and a second polynucleotide strand with an engineered RNA ligase of any one of claims 1-6, 16-21, 28, 29, 34, 35, and 38-41 in presence of a nucleotide substrate under conditions suitable for ligation of the first polynucleotide strand to the second polynucleotide strand, wherein the first polynucleotide strand comprises a ligatable 5′-end and the second polynucleotide strand comprises a 3′-end ligatable to the 5′-end of the first polynucleotide.

66. The method of claim 65, further comprising a third polynucleotide strand, wherein the first polynucleotide strand and second polynucleotide strand hybridize adjacent to one another on the third polynucleotide strand to position the 5′-end of the first polynucleotide strand adjacent to the 3′-end of the second polynucleotide strand.

67. The method of claim 66, wherein the third polynucleotide strand is continuous with the first polynucleotide strand or second polynucleotide strand.

68. The method of claim 67, wherein the third polynucleotide strand is continuous with the first polynucleotide strand and second polynucleotide strand to form a single continuous polynucleotide substrate.

69. The method of claim 66, wherein the third polynucleotide strand comprises a splint or bridging polynucleotide, wherein the 5′-terminal sequence of the first polynucleotide strand and the 3′-terminal sequence of the second polynucleotide strand hybridize adjacent to one another on the splint or bridging polynucleotide to position the 5′-end of the first polynucleotide strand adjacent to the 3′-end of the second polynucleotide strand.

70. The method of claim 66, wherein the first and third polynucleotide strands hybridize to each other to form a first double stranded polynucleotide fragment, and the second polynucleotide strand hybridizes to a fourth polynucleotide strand to form a second double stranded fragment, wherein the first and second double stranded fragments have complementary ends that can base pair to form substrates for the engineered RNA ligase.

71. The method of claim 65, wherein the first polynucleotide strand and/or second polynucleotide strand comprises RNA or a mixture of RNA and DNA.

72. The method of claim 65, wherein the 3′-end of the second polynucleotide strand is a 3′-OH.

73. The method of claim 65, wherein the 5′-end of the first polynucleotide strand is a 5′-phosphate.

74. The method of claim 65, wherein the internucleotide linkage comprises a phosphate linkage.

75. The method of claim 65, wherein the first polynucleotide strand and/or second polynucleotide strand comprise one or more nucleotide analogs, wherein the nucleotide analog comprises a modified nucleobase, modified nucleoside sugar residue, modified nucleobase or nucleobase analog, modified internucleotide linkage, and/or modified 5′-end phosphate group.

76. The method of claim 75, wherein the modified 5′-end phosphate group is a phosphate analog.

77. The method of claim 76, wherein the phosphate analog is a phosphorothioate or monomethylphosphate.

78. The method of claim 75, wherein the modified nucleoside sugar residue is a 2′-O-alkyl, a 2′-fluoro, a 3-D-ribo LNA, or a α-L-ribo-LNA (locked nucleic acids).

79. The method of claim 75, wherein the modified nucleobase or nucleobase analog is xanthine, hypoxanthine, inosine, 7-methylguanine, 2,6-diaminopurine, 5-methylcytosine, 5-hydroxycytosine, 5-bromocytosine, 5-iodocytosine, 2-thiothymine, 5-fluorouracil, 5-bromouracil, 8-bromoguanine, 8-aminoguanine, or 8-aza-7-deazaguanine.

80. The method of claim 75, wherein the internucleotide linkage is a phosphorothioate, phosphoacetate, phosphoramidate, methylphosphonate, or phosphonocarboxylate.

81. The method of claim 65, wherein the nucleotide substrate is ATP or dATP.

82. A kit comprising at least an RNA ligase of any one of claims 1-6, 16-21, 28, 29, 34, 35, and 38-41.

83. The kit of claim 82, further comprising one or more of a buffer, nucleotide substrate, a polynucleotide substrate of the ligase, and/or a ligation enhancer.
